(19) United States
(12) Patent Application Publication
(10) Pub. No.: US 2025/0364089 A1
(43) Pub. Date: Nov. 27, 2025

(54) MACHINE LEARNING ENABLED ENHANCEMENT OF MOLECULAR PROPERTIES

(71) Applicants: Genentech, Inc., South San Francisco, CA (US); F. Hoffmann-La Roche AG, Basel (CH); Hoffmann-La Roche Inc., Little Falls, NJ (US)

(72) Inventors: Vladimir GLIGORIJEVIC, New Brunswick, NJ (US); Andreas LOUKAS, Basel (CH); Natasa TAGASOVSKA, Vaud (CH)

(21) Appl. No.: 19/216,541

(22) Filed: May 22, 2025

Related U.S. Application Data
(60) Provisional application No. 63/650,669, filed on May 22, 2024.

(51) Int. Cl. G16C 20/50; G16C 20/70

(52) U.S. Cl. CPC G16C 20/50; G16C 20/70

(57) ABSTRACT

An input molecule exhibiting a value for one or more properties may be identified. A molecule design computation model may be applied to generate one or more output molecule exhibiting a different value for the one or more properties than the input molecule. The molecule design computation model may generate the one or more output molecules by at least encoding the input molecule to generate an embedding of the input molecule, and decoding the embedding of the input molecule to generate the one or more output molecules. In some cases, the molecule design computation model may generate the one or more output molecules by denoising an input molecule while conditioned on the input molecule. In some cases, the molecule design computation model may operate on a joint representation of the input molecule that combines a linear and a three-dimensional representation of the input molecule.

### CROSS REFERENCE TO RELATED APPLICATION

This application claims priority to U.S. Provisional Application No. 63/650,669, entitled “MACHINE LEARNING ENABLED ENHANCEMENT OF MOLECULAR PROPERTIES” and filed on May 22, 2024, the disclosure of which is incorporated herein by reference in its entirety.

### TECHNICAL FIELD

The subject matter described herein relates generally to molecular design and more specifically to a machine learning based technique for enhancing one or more properties of a molecule.

### INTRODUCTION

A molecule is a group of two more atoms held together by chemical bonds. Molecules form the smallest identifiable unit into which a pure substance can be divided while still retaining the composition and chemical properties of that substance. Various properties of a molecule, including its ability to function as a therapeutic, may be contingent upon the conformation (or three-dimensional structure) of the molecule. One example of a molecule is a small molecule, which is a low-weight compound having a molecular weight between approximately 100 Daltons and 1000 Daltons. Small molecule therapeutics, which modulate biochemical processes to diagnose, treat, and prevent a gamut of illnesses, have been a cornerstone in modern pharmacology due to a number of compelling advantages. For example, small molecule drugs are capable of penetrating cell membranes to reach intracellular targets. Moreover, small molecule drugs are adaptable to a wide variety of therapeutic applications. For instance, a small molecule drug may be formulated as pills and capsules, intravenous or subcutaneous injectables, inhalational medicines, or suppositories. The development of the small molecule drug may further extend to tailoring various pharmacokinetic properties including liberation, absorption, distribution, metabolism, potency, efficacy, phenotypic effects, and excretion.

By contrast, large molecules (also known as biopharmaceuticals, biologicals, or biologics) can range between approximately 3000 Daltons and 150,000 Daltons in molecular weight. Large molecule drugs are often derivatives of natural human proteins, which modulate many essential cellular functions such as enzymatic reactions, transport of molecules, regulation and execution of a number of biological pathways, cell growth, proliferation, nutrient uptake, morphology, motility, intercellular communication, and/or the like. It is common for a single large molecule to have more than 1,300 amino acid residues, which are linked by peptide bonds to form one or more polypeptide. Due to their size and complexity, large molecule drugs are recombinantly produced by engineered cells instead of being chemically synthesized like the majority of small molecule drugs. Moreover, large molecule therapeutics are usually delivered through injection or infusion due to the ineffectiveness of oral administration. The development of a large molecule drug may entail designing one or more sequences of amino acid residues capable of binding to a target (e.g., a protein, a nucleic acid, and/or the like) with sufficient specificity and absent undesired traits such as immunogenicity, self-association, instability, and/or the like.

### SUMMARY

Systems, methods, and articles of manufacture, including computer program products, are provided for machine learning enabled enhancement of molecular properties. One salient aspect of drug design is the improvement or enhancement of properties of interest including, for example, drug-like properties such as binding affinity, specificity, biological activity, developability, and/or the like. In some cases, one or more properties of an input molecule, such as a chemical compound, a peptide, a protein, or a nucleic acid, may be improved by at least applying a molecule design computation model trained to generate one or more candidate molecules, each of which exhibiting a different value for the one or more properties than the input molecule. For example, in some cases, the molecule design computation model may generate an output molecule by at least encoding the input molecule to generate an embedding of the input molecule before the embedding of the input molecule is decoded to generate an output molecule.

In some cases, the molecule design computation model may operate on a linear (or one-dimensional) representation, a two dimensional representation, and/or a three-dimensional representation of the input molecule. In some cases, the molecule design computation model may operate on a joint representation of the input molecule that combines, for example, a linear (or one-dimensional) representation of the input molecule with a higher-dimensional representation of the input molecule, such as a two dimensional representation or a three-dimensional representation of the input molecule. In some cases, the molecule design computation model may be trained on a matched dataset containing one or more molecule pairs exhibiting different values for one or more properties of interest. In some cases, the molecule design computation model may be trained to approximate the gradient of the value of the one or more properties (e.g., a function that predicts the value of the one or more properties present in a molecule). For example, in some cases, the molecule design computation model may approximate the gradient by at least being trained to recover, from the embedding of the molecule with an inferior value for the one or more properties in each molecule pair, the other molecule with a superior value for the one or more properties. Accordingly, in some cases, the generation of one or more output molecules may be guided by this gradient such that the function outputs, for each successive output molecule, a superior value for the one or more properties. Alternatively, the molecule design computation model may be trained to approximate a data distribution (or matched distribution) of molecule pairs such that output molecules exhibiting a different value for the one or more properties may be generated by sampling from the data distribution. For instance, in some cases, the molecule design computation model may be trained to recover, from a noise molecule, the molecule in each molecule pair with the superior value for the one or more properties while conditioned on the other molecule in the molecule pair with the inferior value for the one or more properties.

In some cases, the output of the molecule design computation model may include one or more output molecules exhibiting modifications, including compositional modifications and/or conformational modifications, relative to the input molecule. For example, in some cases, these modifications may engender the difference in the value of the one or more properties between the input molecule and each output molecule. In some cases, instead of the one or more output molecules, the output of the molecule design computation model may indicate one or more modifications to the input molecule that changes the value of the one or more properties present in the input molecule. For instance, in some cases, the output of the molecule design computation model may be a multinomial distribution of the possible composition and/or conformation of the output molecule such that individual output molecules may be generated by sampling from the multinomial distribution.

In one aspect, there is provided a system for machine learning enabled enhancement of molecular properties. The system may include at least one data processor and at least one memory. The at least one memory may store instructions that result in operations when executed by the at least one data processor. The operations may include: identifying an input molecule exhibiting a value for one or more properties; and applying a molecule design computation model to generating one or more output molecules exhibiting a different value for the one or more properties than the input molecule, wherein the molecule design computation model generates the one or more molecules by at least encoding the input molecule to generate an embedding of the input molecule, and decoding the embedding of the input molecule to generate the one or more output molecules.

In another aspect, there is provided a computer-implemented method for machine learning enabled enhancement of molecular properties. The method may include: identifying an input molecule exhibiting a value for one or more properties; and applying a molecule design computation model to generating one or more output molecules exhibiting a different value for the one or more properties than the input molecule, wherein the molecule design computation model generates the one or more molecules by at least encoding the input molecule to generate an embedding of the input molecule, and decoding the embedding of the input molecule to generate the one or more output molecules.

In another aspect, there is provided a computer program product for machine learning enabled enhancement of molecular properties. The computer program product may include a non-transitory computer readable medium storing instructions that result in operations when executed by at least one data processor. The operations may include: identifying an input molecule exhibiting a value for one or more properties; and applying a molecule design computation model to generating one or more output molecules exhibiting a different value for the one or more properties than the input molecule, wherein the molecule design computation model generates the one or more molecules by at least encoding the input molecule to generate an embedding of the input molecule, and decoding the embedding of the input molecule to generate the one or more output molecules.

In some variations, one or more features disclosed herein including the following features can optionally be included in any feasible combination.

In some variations, the generating one or more output molecules includes: applying the molecule design computation model to generate an output molecule; determining that the output molecule fails to satisfy one or more criteria; and in response to determining that the output molecule fails to satisfy the one or more criteria, applying the molecule design computation model to generate an additional output molecule.

In some variations, the molecule design computation model generates the additional output molecule by at least encoding the output molecule to generate an embedding of the output molecule, and decoding the embedding of the output molecule to generate the additional output molecule.

In some variations, the one or more criteria include at least one of (i) a proximity measure between the input molecule and the output molecule satisfying a first threshold, and (ii) a difference in the value of the one or more properties present in the input molecule and the different value of the one or more properties present in the output molecule satisfying a second threshold.

In some variations, a plurality of molecule pairs are identified for inclusion in a training dataset. Each molecule pair includes two molecules exhibiting different values for the one or more properties. The molecule design computation model is trained based at least on the training dataset. The molecule design computation model is trained to generate, based at least on a first molecule in each molecule pair, a reconstruction of a second molecule in each molecule pair.

In some variations, the molecule design computation model is trained, based at least on the training dataset, to at least encode the first molecule to generate an embedding of the first molecule, and decode the embedding of the first molecule to generate the reconstruction of the second molecule.

In some variations, the training of the molecule design computation model includes reducing a reconstruction loss associated with a difference between the second molecule and the reconstruction of the second molecule generated by the molecule design computation model.

In some variations, the training of the molecule design computation model includes imposing a monotonicity constraint by at least ensuring that a first output of the molecule design computation model operating on the first molecule is greater than a second output of the molecule design computation model operating on the second molecule where the first molecule is greater than the second molecule.

In some variations, each molecule pair is identified by at least identifying, based at least on one or more criteria being satisfied, the first molecule as a match for the second molecule.

In some variations, the one or more criteria are determined to be satisfied based at least on a proximity measure between the first molecule and the second molecule satisfying one or more thresholds.

In some variations, the proximity measure includes one or more of an edit distance, a structural similarity, an amino acid substitution matrix, a chemical similarity coefficient, a Euclidean distance, atomic coordinates, torsion angles, and an embedding of each of the first molecule and the second molecule.

In some variations, the one or more criteria are determined to be satisfied based at least on a difference in a value of the one or more properties present in the first molecule and a value of the one or more properties present in the second molecule satisfying one or more thresholds.

In some variations, the one or more properties include a first property and a second property.

In some variations, the one or more criteria are determined to be satisfied based at least on a difference in a respective value of either the first property or a second property present in each of the first molecule and the second molecule satisfying one or more thresholds.

In some variations, a multivariate rank indicative of a difference in a combination of the first property and the second property is determined for each of the first molecule and the second molecule. The one or more criteria are determined to be satisfied based at least on a difference in a respective multivariate rank of the first molecule and the second molecule satisfying one or more thresholds.

In some variations, a respective multivariate rank of the first molecule and the second molecule is determined by applying one or more of an expected hypervolume improvement (EHVI), a noisy expected hypervolume improvement (NEHVI), a cumulative distribution function (CDF), a Pareto efficient global optimization (ParEGO), a max-value entropy search method (MESMO), or a joint entropy search (JES).

In some variations, the first property and the second property comprise a different one of binding affinity, binding specificity, hydrophobicity, size of electrical charge patches, angle delta, angle length, immunogenicity, and presence of liability motifs.

In some variations, the molecule design computation model comprises an encoder that encodes the input molecule and a decoder that decodes the embedding of the input molecule.

In some variations, the molecule design computation model comprises an autoencoder including an encoder coupled with a decoder.

In some variations, each output molecule of the one or more output molecules exhibits one or more compositional modifications and/or conformational modifications relative to the input molecule.

In some variations, the input molecule comprises a protein sequence and the output molecule comprises a different protein sequence.

In some variations, the input molecule comprises a nucleic acid molecule and the output molecule comprises a nucleic acid molecule having a different sugar-phosphate backbone than the input molecule.

In some variations, the input molecule comprises a chemical compound and the output molecule comprises a chemical compound having one or more different functional groups than the input molecule.

In some variations, the identifying of the input molecule includes identifying a representation of the input molecule. The representation of the input molecule includes one or more of a real data vector, a point cloud representation, an atomic density field representation, an image pixel representation, or a tokenized sequence molecule representation.

In some variations, the molecule design computation model generates an output comprising a multinomial distribution of a plurality of possible composition and/or a plurality of conformation of the one or more output molecules.

In some variations, each output molecule of the one or more output molecules is generated by at least sampling from the multinomial distribution.

In some variations, the multinomial distribution includes, for each possible position in a protein sequence, a probability of the position being occupied by each of a plurality of possible amino acid residues.

In some variations, the sampling from the multinomial distribution includes determining, based on the multinomial distribution, a type of amino acid residue occupying each position in a corresponding protein sequence. The type of amino acid residue determined to occupy a position in the corresponding protein sequences comprises a type of amino acid residue whose probability of occupying the position satisfies one or more thresholds.

In another aspect, there is provided a system for generating a matched dataset for training a molecule design computation model to enhance molecular properties. The system may include at least one data processor and at least one memory. The at least one memory may store instructions that result in operations when executed by the at least one data processor. The operations may include: identifying, for inclusion in a matched dataset, a plurality of molecule pairs, wherein each molecule pair of the plurality of molecule pairs include two molecules exhibiting different values for one or more properties; training, based at least on the matched dataset, a molecule design computation model to generate, based at least on a first molecule in each molecule pair, a reconstruction of a second molecule in each molecule pair, wherein the molecule design computation model is trained to generate the reconstruction of the second molecule by at least encoding the first molecule to generate an embedding of the first molecule, and decoding the embedding of the first molecule to generate the reconstruction of the second molecule; and applying the molecule design computation model to generate, based at least on an input molecule, one or more output molecules having a different value for the one or more properties than the input molecule.

In another aspect, there is provided a computer-implemented method for generating a matched dataset for training a molecule design computation model to enhance molecular properties. The method may include: identifying, for inclusion in a matched dataset, a plurality of molecule pairs, wherein each molecule pair of the plurality of molecule pairs include two molecules exhibiting different values for one or more properties; training, based at least on the matched dataset, a molecule design computation model to generate, based at least on a first molecule in each molecule pair, a reconstruction of a second molecule in each molecule pair, wherein the molecule design computation model is trained to generate the reconstruction of the second molecule by at least encoding the first molecule to generate an embedding of the first molecule, and decoding the embedding of the first molecule to generate the reconstruction of the second molecule; and applying the molecule design computation model to generate, based at least on an input molecule, one or more output molecules having a different value for the one or more properties than the input molecule.

In another aspect, there is provided a computer program product for generating a matched dataset for training a molecule design computation model to enhance molecular properties. The computer program product may include a non-transitory computer readable medium storing instructions that result in operations when executed by at least one data processor. The operations may include: identifying, for inclusion in a matched dataset, a plurality of molecule pairs, wherein each molecule pair of the plurality of molecule pairs include two molecules exhibiting different values for one or more properties; training, based at least on the matched dataset, a molecule design computation model to generate, based at least on a first molecule in each molecule pair, a reconstruction of a second molecule in each molecule pair, wherein the molecule design computation model is trained to generate the reconstruction of the second molecule by at least encoding the first molecule to generate an embedding of the first molecule, and decoding the embedding of the first molecule to generate the reconstruction of the second molecule; and applying the molecule design computation model to generate, based at least on an input molecule, one or more output molecules having a different value for the one or more properties than the input molecule.

In some variations, one or more features disclosed herein including the following features can optionally be included in any feasible combination.

In some variations, each molecule pair is identified by at least identifying, based at least on one or more criteria, the first molecule as a match for the second molecule.

In some variations, the one or more criteria are determined to be satisfied based at least on a proximity measure between the first molecule and the second molecule satisfying one or more thresholds.

In some variations, the proximity measure includes one or more of an edit distance, a structural similarity, an amino acid substitution matrix, a chemical similarity coefficient, a Euclidean distance, atomic coordinates, torsion angles, and an embedding of each of the first molecule and the second molecule.

In some variations, the one or more criteria are determined to be satisfied based at least on a difference in a value of the one or more properties present in the first molecule and a value of the one or more properties present in the second molecule satisfying one or more thresholds.

In some variations, the one or more properties include a first property and a second property.

In some variations, the one or more criteria are determined to be satisfied based at least on a difference in a respective value of either the first property or a second property present in each of the first molecule and the second molecule satisfying one or more thresholds.

In some variations, a multivariate rank indicative of a difference in a combination of the first property and the second property is determined for each of the first molecule and the second molecule. The one or more criteria are determined to be satisfied based at least on a difference in a respective multivariate rank of the first molecule and the second molecule satisfying one or more thresholds.

In some variations, a respective multivariate rank of the first molecule and the second molecule is determined by applying one or more of an expected hypervolume improvement (EHVI), a noisy expected hypervolume improvement (NEHVI), a cumulative distribution function (CDF), a Pareto efficient global optimization (ParEGO), a max-value entropy search method (MESMO), or a joint entropy search (JES).

In some variations, the first property and the second property comprise a different one of binding affinity, binding specificity, hydrophobicity, size of electrical charge patches, angle delta, angle length, immunogenicity, and presence of liability motifs.

In another aspect, there is provided a system for implicitly guided generation of molecules by matching data points. The system may include at least one data processor and at least one memory. The at least one memory may store instructions that result in operations when executed by the at least one data processor. The operations may include: identifying, for inclusion in a matched dataset, a plurality of molecule pairs, wherein each molecule pair of the plurality of molecule pairs include two molecules exhibiting different values for a property; training, based at least on the matched dataset, a molecule design computation model; and applying the molecule design computation model to generate one or more output molecules exhibiting a different value for property than an input molecule, wherein the molecule design computation model generates the one or more output molecules by at least denoising a noise molecule while conditioned on the input molecule.

In another aspect, there is provided a computer-implemented method for implicitly guided generation of molecules by matching data points. The method may include: identifying, for inclusion in a matched dataset, a plurality of molecule pairs, wherein each molecule pair of the plurality of molecule pairs include two molecules exhibiting different values for a property; training, based at least on the matched dataset, a molecule design computation model; and applying the molecule design computation model to generate one or more output molecules exhibiting a different value for property than an input molecule, wherein the molecule design computation model generates the one or more output molecules by at least denoising a noise molecule while conditioned on the input molecule.

In another aspect, there is provided a computer program product for implicitly guided generation of molecules by matching data points. The computer program product may include a non-transitory computer readable medium storing instructions that result in operations when executed by at least one data processor. The operations may include: identifying, for inclusion in a matched dataset, a plurality of molecule pairs, wherein each molecule pair of the plurality of molecule pairs include two molecules exhibiting different values for a property; training, based at least on the matched dataset, a molecule design computation model; and applying the molecule design computation model to generate one or more output molecules exhibiting a different value for property than an input molecule, wherein the molecule design computation model generates the one or more output molecules by at least denoising a noise molecule while conditioned on the input molecule.

In some variations, one or more features disclosed herein including the following features can optionally be included in any feasible combination.

In some variations, the molecule design computation model is trained, based at least on the matched dataset, to approximate a data distribution of molecule pairs in which one molecule in each molecule pair exhibits a superior value for the property than another molecule in a same molecule pair.

In some variations, the molecule design computation model generates the one or more output molecules by at least sampling each output molecule from the data distribution.

In some variations, the generating the one or more output molecules includes: applying the molecule design computation model to generate an output molecule; determining that the output molecule fails to satisfy one or more criteria; and in response to determining that the output molecule fails to satisfy the one or more criteria, applying the molecule design computation model to generate an additional output molecule.

In some variations, the molecule design computation model generates the additional output molecule by at least denoising the noise molecule while conditioned on the output molecule.

In some variations, the one or more criteria include at least one of (i) a proximity measure between the input molecule and the output molecule satisfying a first threshold, and (ii) a difference in the value of the property present in the input molecule and the different value of the property present in the output molecule satisfying a second threshold.

In some variations, a plurality of molecule pairs are identified for inclusion in a matched dataset. Each molecule pair includes two molecules exhibiting different values for the property. The molecule design computation model is trained, based at least on the matched dataset, to recover one molecule in each molecule pair by at least denoising the noise molecule while conditioned on another molecule in each molecule pair

In some variations, the training of the molecule design computation model includes reducing a difference between the one molecule and a reconstruction of the one molecule generated by the molecule design computation model denoising the noise molecule.

In some variations, each molecule pair includes a first molecule and a second molecule. Each molecule pair is identified by at least identifying, based at least on one or more criteria being satisfied, the first molecule as a match for the second molecule.

In some variations, the one or more criteria are determined to be satisfied based at least on a proximity measure between the first molecule and the second molecule satisfying one or more thresholds.

In some variations, the proximity measure includes one or more of an edit distance, a structural similarity, an amino acid substitution matrix, a chemical similarity coefficient, a Euclidean distance, atomic coordinates, torsion angles, and an embedding of each of the first molecule and the second molecule.

In some variations, the one or more criteria are determined to be satisfied based at least on a difference in a value of the property present in the first molecule and a value of the property present in the second molecule satisfying one or more thresholds.

In some variations, the two molecules comprising each molecule pair of the plurality of molecule pairs exhibit different values for the property and/or an additional property.

In some variations, the one or more criteria are determined to be satisfied based at least on a difference in a respective value of either the property or the additional property present in each of the first molecule and the second molecule satisfying one or more thresholds.

In some variations, a multivariate rank indicative of a difference in a combination of the property and the additional property is determined for each of the first molecule and the second molecule. The one or more criteria are determined to be satisfied based at least on a difference in a respective multivariate rank of the first molecule and the second molecule satisfying one or more thresholds.

In some variations, a respective multivariate rank of the first molecule and the second molecule is determined by applying one or more of an expected hypervolume improvement (EHVI), a noisy expected hypervolume improvement (NEHVI), a cumulative distribution function (CDF), a Pareto efficient global optimization (ParEGO), a max-value entropy search method (MESMO), or a joint entropy search (JES).

In some variations, the property and the additional property comprise a different one of binding affinity, binding specificity, hydrophobicity, size of electrical charge patches, angle delta, angle length, immunogenicity, and presence of liability motifs.

In some variations, the molecule design computation model comprises a conditional denoiser that denoises the noise molecule while conditioned on the input molecule.

In some variations, the molecule design computation model comprises a variational autoencoder, a flow matching model, or a score-based generative model.

In some variations, each output molecule of the one or more output molecules is generated to exhibit one or more compositional modifications and/or conformational modifications relative to the input molecule.

In some variations, the input molecule comprises a protein sequence and the output molecule comprises a different protein sequence.

In some variations, the input molecule comprises a nucleic acid molecule and the output molecule comprises a nucleic acid molecule having a different sugar-phosphate backbone than the input molecule.

In some variations, the input molecule comprises a chemical compound and the output molecule comprises a chemical compound having one or more different functional groups than the input molecule.

In some variations, the molecule design computation model is applied to a representation of the input molecule to generate a representation of each output molecule of the one or more output molecules.

In some variations, the representation of the input molecule and the representation of each output molecule comprise one or more of a real data vector, a point cloud representation, an atomic density field representation, an image pixel representation, or a tokenized sequence molecule representation.

In some variations, one or more pseudo-matched molecule pairs are generated. The molecule design computation model is trained based at least on the one or more pseudo-matched molecule pairs.

In some variations, each pseudo-matched molecule pair of the one or more pseudo-matched molecule pairs is generated by at least selecting, from the matched dataset, a molecule pair including a first molecule and a second molecule. The molecule design computation model is applied to generate a reconstruction of the first molecule from the molecule pair by at least denoising a noise molecule while conditioned on the second molecule from the molecule pair, and generating each pseudo-matched molecule pair to include the second molecule from the molecule pair and the reconstruction of the first molecule.

In some variations, each pseudo-matched molecule pair of the one or more pseudo-matched molecule pairs is further generated by at least determining an edit distance between the second molecule and the reconstruction of the first molecule, determining a difference in a value of the property present in the second molecule and a value of the property present in the reconstruction of the first molecule, and generating each pseud-matched molecule pair to include the second molecule and the reconstruction of the first molecule based at least on the edit distance and the difference in a respective value of the property satisfying one or more thresholds.

In another aspect, there is provided a system for structure-informed machine learning enabled enhancement of molecular properties. The system may include at least one data processor and at least one memory. The at least one memory may store instructions that result in operations when executed by the at least one data processor. The operations may include: identifying an input molecule exhibiting a value for a property; generating a joint representation of the input molecule that combines a linear representation of the input molecule with a three-dimensional representation of the input molecule; and applying a molecule design computation model to determine, based at least on the joint representation of the input molecule, a joint representation of an output molecule exhibiting a different value for the property than the value of the property present in the input molecule.

In another aspect, there is provided a computer-implemented method for structure-informed machine learning enabled enhancement of molecular properties. The method may include: identifying an input molecule exhibiting a value for a property; generating a joint representation of the input molecule that combines a linear representation of the input molecule with a three-dimensional representation of the input molecule; and applying a molecule design computation model to determine, based at least on the joint representation of the input molecule, a joint representation of an output molecule exhibiting a different value for the property than the value of the property present in the input molecule.

In another aspect, there is provided a computer program product for structure-informed machine learning enabled enhancement of molecular properties. The computer program product may include a non-transitory computer readable medium storing instructions that result in operations when executed by at least one data processor. The operations may include: identifying an input molecule exhibiting a value for a property; generating a joint representation of the input molecule that combines a linear representation of the input molecule with a three-dimensional representation of the input molecule; and applying a molecule design computation model to determine, based at least on the joint representation of the input molecule, a joint representation of an output molecule exhibiting a different value for the property than the value of the property present in the input molecule.

In some variations, one or more features disclosed herein including the following features can optionally be included in any feasible combination.

In some variations, the input molecule comprises a protein molecule. The joint representation of the input molecule combines an amino acid sequence of the input molecule and structural context information.

In some variations, the structural context information identifies, for each amino acid residue in the input molecule, one or more other amino acid residues that are located within a threshold distance in three-dimensional space.

In some variations, the structural context information comprises an adjacency matrix.

In some variations, the joint representation of the output molecule is decoded to generate a linear representation of the output molecule.

In some variations, the molecule design computation model generates the joint representation of the output molecule by at least encoding the joint representation of the input molecule to generate an embedding of the input molecule, and decoding the embedding of the input molecule to generate the joint representation of the output molecule.

In some variations, the molecule design computation model generates the joint representation of the output molecule by at least denoising a noise molecule while conditioned on the joint representation of the input molecule.

In some variations, the output molecule is determined to fail to satisfy one or more criteria. In response to determining that the output molecule fails to satisfy the one or more criteria, the molecule design computation model is applied to generate, based at least on the joint representation of the output molecule, a joint representation of an additional output molecule

In some variations, the molecule design computation model is applied to generate one or more additional output molecules until the one or more criteria are satisfied.

In some variations, wherein the one or more criteria include at least one of (i) a proximity measure between the input molecule and the output molecule satisfying a first threshold, and (ii) a difference in the value of the property present in the input molecule and the different value of the property present in the output molecule satisfying a second threshold.

In some variations, a plurality of molecule pairs are identified for inclusion in a matched dataset. Each molecule pair includes two molecules exhibiting different values for the property. The molecule design computation model is trained based at least on the matched dataset. The molecule design computation model is trained to generate, based at least on a joint representation of the first molecule in each molecule pair, a joint representation that corresponds to a joint representation of the second molecule in each molecule pair.

In some variations, the training of the molecule design computation model includes reducing a difference between the joint representation of the second molecule and the joint representation generated by the molecule design computation model.

In some variations, the identifying the plurality of molecule pairs includes identifying each molecule pair by at least determining, based at least on one or more criteria being satisfied, the first molecule as a match for the second molecule.

In some variations, the one or more criteria are determined to be satisfied based at least on a proximity measure between the first molecule and the second molecule satisfying one or more thresholds.

In some variations, the proximity measure includes one or more of an edit distance, a structural similarity, an amino acid substitution matrix, a chemical similarity coefficient, a Euclidean distance, atomic coordinates, torsion angles, and an embedding of each of the first molecule and the second molecule.

In some variations, the one or more criteria are determined to be satisfied based at least on a difference in a value of the property present in the first molecule and a value of the property present in the second molecule satisfying one or more thresholds.

In some variations, the one or more criteria are determined to be satisfied based at least on a value of the property and/or a value of an additional property present in each of the first molecule and the second molecule.

In some variations, the one or more criteria are determined to be satisfied based at least on a difference in a respective value of either the property or the additional property present in each of the first molecule and the second molecule satisfying one or more thresholds.

In some variations, a multivariate rank indicative of a difference in a combination of the property and the additional property is determined for each of the first molecule and the second molecule. The one or more criteria are determined to be satisfied based at least on a difference in a respective multivariate rank of the first molecule and the second molecule satisfying one or more thresholds.

In some variations, the respective multivariate rank of the first molecule and the second molecule is determined by applying one or more of an expected hypervolume improvement (EHVI), a noisy expected hypervolume improvement (NEHVI), a cumulative distribution function (CDF), a Pareto efficient global optimization (ParEGO), a max-value entropy search method (MESMO), or a joint entropy search (JES).

In some variations, the property and the additional property comprise a different one of binding affinity, binding specificity, hydrophobicity, size of electrical charge patches, angle delta, angle length, immunogenicity, and presence of liability motifs.

In some variations, the molecule design computation model is applied to perform one-shot optimization of the input molecule whose amino acid sequence is out-of-distribution (OOD) of the matched dataset.

In some variations, each output molecule of the one or more output molecules exhibits one or more compositional modifications and/or conformational modifications relative to the input molecule.

In some variations, the input molecule comprises a protein sequence and the output molecule comprises a different protein sequence.

In some variations, the input molecule comprises a nucleic acid molecule and the output molecule comprises a nucleic acid molecule having a different sugar-phosphate backbone than the input molecule.

In some variations, the input molecule comprises a chemical compound and the output molecule comprises a chemical compound having one or more different functional groups than the input molecule.

In some variations, the molecule design computation model comprises an encoder and a decoder.

In some variations, the molecule design computation model comprises a graph transformer.

In one aspect, there is provided a system for machine learning enabled enhancement of molecular properties. The system may include at least one data processor and at least one memory. The at least one memory may store instructions that result in operations when executed by the at least one data processor. The operations may include: training, based at least on a matched dataset, a first instance of a molecule design computation model, where each molecule pair in the matched dataset includes two sample molecules exhibiting different values for a property, and where the first instance of the molecule design computation model is trained to generate, based at least on an input molecule, one or more output molecules exhibiting a different value for the property than a value of the property present in the input molecule; applying the first trained instance of the molecule design computation model to generate a first pseudo-matched dataset, where each pseudo-matched molecule pair in the first pseudo-matched dataset includes a sample molecule from the matched dataset paired with an output molecule generated by the first trained instance of the molecule design computation model operating on the sample molecule; and training, based at least on the matched dataset and the first pseudo-matched dataset, a second instance of the molecule design computation model to generate, based at least on the input molecule, the one or more output molecules exhibiting the different value for the property than the value of the property of the input molecule.

In another aspect, there is provided a computer-implemented method for machine learning enabled enhancement of molecular properties. The method may include: training, based at least on a matched dataset, a first instance of a molecule design computation model, where each molecule pair in the matched dataset includes two sample molecules exhibiting different values for a property, and where the first instance of the molecule design computation model is trained to generate, based at least on an input molecule, one or more output molecules exhibiting a different value for the property than a value of the property present in the input molecule; applying the first trained instance of the molecule design computation model to generate a first pseudo-matched dataset, where each pseudo-matched molecule pair in the first pseudo-matched dataset includes a sample molecule from the matched dataset paired with an output molecule generated by the first trained instance of the molecule design computation model operating on the sample molecule; and training, based at least on the matched dataset and the first pseudo-matched dataset, a second instance of the molecule design computation model to generate, based at least on the input molecule, the one or more output molecules exhibiting the different value for the property than the value of the property of the input molecule.

In another aspect, there is provided a computer program product for machine learning enabled enhancement of molecular properties. The computer program product may include a non-transitory computer readable medium storing instructions that result in operations when executed by at least one data processor. The operations may include: training, based at least on a matched dataset, a first instance of a molecule design computation model, where each molecule pair in the matched dataset includes two sample molecules exhibiting different values for a property, and where the first instance of the molecule design computation model is trained to generate, based at least on an input molecule, one or more output molecules exhibiting a different value for the property than a value of the property present in the input molecule; applying the first trained instance of the molecule design computation model to generate a first pseudo-matched dataset, where each pseudo-matched molecule pair in the first pseudo-matched dataset includes a sample molecule from the matched dataset paired with an output molecule generated by the first trained instance of the molecule design computation model operating on the sample molecule; and training, based at least on the matched dataset and the first pseudo-matched dataset, a second instance of the molecule design computation model to generate, based at least on the input molecule, the one or more output molecules exhibiting the different value for the property than the value of the property of the input molecule.

In some variations, one or more features disclosed herein including the following features can optionally be included in any feasible combination.

In some variations, the generating the first pseudo-matched dataset further includes applying the first trained instance of the molecule design computation model to generate, based at least on the sample molecule from the matched dataset, the output molecule, and generating, for inclusion in the first pseudo-matched dataset, the pseudo-matched molecule pair including the sample molecule and the output molecule.

In some variations, the generating the first pseudo-matched dataset further includes determining that the sample molecule and the output molecule are counterfactual molecules exhibiting (i) a threshold similarity in molecular composition and/or molecular conformation, and (ii) a threshold difference in a respective value of the property, and upon determining that the sample molecule and the output molecule are counterfactual molecules, generating the pseudo-matched molecule pair to include the sample molecule and the output molecule.

In some variations, the second trained instance of the molecule design computation model is applied to generate a second pseudo-matched dataset. Each pseudo-matched molecule pair in the second pseudo-matched dataset includes a sample molecule from the matched dataset paired with an output molecule generated by the second trained instance of the molecule design computation model operating on the sample molecule. A third instance of the molecule design computation model is trained based at least on the matched dataset, the first pseudo-matched dataset, and the second pseudo-matched dataset. The third instance of the molecule design computation model is trained to generate, based at least on the input molecule, the one or more output molecules exhibiting the different value for the property than the input molecule.

In some variations, the second trained instance of the molecule design computation model is applied to generate, based at least on the input molecule, the one or more output molecules exhibiting the different value for the property than the input molecule.

In some variations, the second trained instance of the molecule design computation model generates the one or more output molecules by at least encoding the input molecule to generate an embedding of the input molecule, and decoding the embedding of the input molecule to generate each output molecule to exhibit the different value for the property than the input molecule.

In some variations, the second trained instance of the molecule design computation model generates the one or more output molecules by at least denoising a noise molecule while conditioned on the input molecule.

In some variations, the second trained instance of the molecule design computation model generates the one or more output molecules by at least generating, based at least on the input molecule, an output molecule; determining that the output molecule fails to satisfy one or more criteria; and in response to determining that the output molecule fails to satisfy the one or more criteria, generating, based at least on the output molecule, an additional output molecule.

In some variations, the second trained instance of the molecule design computation model is applied to generate one or more additional output molecules until the one or more criteria are satisfied.

In some variations, the one or more criteria include at least one of (i) a proximity measure between the input molecule and the output molecule satisfying a first threshold, and (ii) a difference in the value of the property present in the input molecule and the different value of the property present in the output molecule satisfying a second threshold.

In some variations, the first instance of the molecule design computation model and the second instance of the molecule design computation model are trained to approximate a gradient of the property.

In some variations, the first trained instance of the molecule design computation model and the second trained instance of the molecule design computation model generate the one or more output molecules with guidance from the gradient.

In some variations, the first instance of the molecule design computation model and the second instance of the molecule design computation model are trained to approximate a data distribution of a plurality of molecule pairs in which one molecule in each molecule pair exhibits a superior value for the property than the other molecule in the molecule pair.

In some variations, the first trained instance of the molecule design computation model and the second trained instance of the molecule design computation model generate the one or more output molecules by at least sampling each output molecule from the data distribution.

In some variations, the input molecule comprises a protein molecule. The first instance of the molecule design computation model and the second instance of the molecule design computation model are trained to operate on a joint representation of the input molecule that combines an amino acid sequence of the input molecule and structural context information.

In some variations, the structural context information identifies, for each amino acid residue in the input molecule, one or more other amino acid residues that are located within a threshold distance in three-dimensional space.

In some variations, the matched dataset is generated by at least identifying the sample molecule and a different sample molecule as counterfactual molecules exhibiting (i) a threshold similarity in molecular composition and/or molecular conformation, and (ii) a threshold difference in a respective value of the property, and upon determining that the sample molecule and the different sample molecule are counterfactual molecules, generating, for inclusion in the matched dataset, a molecule pair including the sample molecule and the different sample molecule.

In some variations, the sample molecule and the different sample molecule are identified as counterfactual molecules based on the respective value of the property and/or an additional property.

In some variations, the sample molecule and the different sample molecule are identified as counterfactual molecules based at least on a difference in the respective value of either the property or the additional property present in each molecule satisfying one or more thresholds.

In some variations, a multivariate rank indicative of a difference in a combination of the property and the additional property is determined for each of the sample molecule and the different sample molecule. The sample molecule and the different sample molecule are identified as counterfactual molecules based at least on a difference in a respective multivariate rank of each molecule satisfying one or more thresholds.

In some variations, the respective multivariate rank of the first molecule and the second molecule is determined by applying one or more of an expected hypervolume improvement (EHVI), a noisy expected hypervolume improvement (NEHVI), a cumulative distribution function (CDF), a Pareto efficient global optimization (ParEGO), a max-value entropy search method (MESMO), or a joint entropy search (JES).

In some variations, the property and the additional property comprise a different one of binding affinity, binding specificity, hydrophobicity, size of electrical charge patches, angle delta, angle length, immunogenicity, and presence of liability motifs.

In some variations, each output molecule of the one or more output molecules exhibits one or more compositional modifications and/or conformational modifications relative to the input molecule.

In some variations, the input molecule comprises a protein sequence and the output molecule comprises a different protein sequence.

In some variations, the input molecule comprises a nucleic acid molecule and the output molecule comprises a nucleic acid molecule having a different sugar-phosphate backbone than the input molecule.

In some variations, the input molecule comprises a chemical compound and the output molecule comprises a chemical compound having one or more different functional groups than the input molecule.

In some variations, the molecule design computation model comprises an autoencoder or a graph transformer.

In some variations, the molecule design computation model comprises a variational autoencoder, a flow matching model, or a score-based generative model.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the design of large molecules such as protein molecules, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a molecule design system, in accordance with some example embodiments;

FIG. 2A depicts a flowchart illustrating an example of a process for machine learning enabled enhancement of molecular properties, in accordance with some example embodiments;

FIG. 2B depicts a flowchart illustrating an example of a process for generating a matched dataset, in accordance with some example embodiments;

FIG. 2C depicts a flowchart illustrating an example of a process for iteratively training a molecule design computation model on pseudo-matched molecule pairs, in accordance with some example embodiments;

FIG. 3A depicts a flowchart illustrating an example of a process for machine learning enhancement of one or more molecular properties, in accordance with some example embodiments;

FIG. 3B depicts a flowchart illustrating another example of a process for machine learning enhancement of one or more molecular properties, in accordance with some example embodiments;

FIG. 3C depicts a flowchart illustrating another example of a process for machine learning enhancement of one or more molecular properties, in accordance with some example embodiments;

FIG. 4 depicts a flowchart illustrating an example of a process for iterative enhancement of molecular properties, in accordance with some example embodiments;

FIG. 5A depicts a schematic diagram illustrating an example of a process for training a molecular design computation model based on one or more molecule pairs in a matched dataset, in accordance with some example embodiments;

FIG. 5B depicts a schematic diagram illustrating an example of a process for iterative optimization of molecular properties, in accordance with some example embodiments;

FIG. 5C depicts a schematic diagram illustrating an example of a process for machine learning enabled enhancement of molecular properties, in accordance with some example embodiments;

FIG. 6A depicts a schematic diagram illustrating a comparison of an example of an optimization task with implicitly guided property optimization and another example of an optimization task with explicit guidance, in accordance with some example embodiments;

FIG. 6B depicts a schematic diagram illustrating a comparison of an example of an optimization task with independent and identically distributed (IID) seed molecules and another example of an optimization task with out-of-distribution (OOD) seed molecules, in accordance with some example embodiments;

FIG. 7A depicts graphs illustrating a comparison of the performance different molecule design computation models for in silico/computational optimization of different molecular properties including hydrophobicity, charge, chemical liabilities, bond lengths, and bond angles, in accordance with some example embodiments;

FIG. 7B depicts a graph illustrating a comparison of the wet-lab binding affinity measurements of molecule designs that have been computationally optimized across three rounds and for five different targets, in accordance with some example embodiments;

FIG. 7C depicts a schematic diagram illustrating an example of a molecule design that has been computationally optimized to exhibit 30× stronger binding affinity than the lead molecule, in accordance with some example embodiments; and

FIG. 8A depicts an illustrative two-dimensional dataset and a comparison of the results of unguided conditional generation, implicitly guided property optimization, and implicitly guided conditional generation with increasing values of the property, in accordance with some example embodiments;

FIG. 8B depicts graphs illustrating the conditional expectation of output molecules with improved property values, the probability of finding a molecule design with superior property values, and a comparison of the property values of the input molecule and that of superior output molecules, in accordance with some example embodiments;

FIG. 8C depicts graphs illustrating a comparison of the performance of a conditional generator model conditioned on a matched dataset and that of a unconditional generator model, in accordance with some example embodiments;

FIG. 9A depicts a schematic diagram illustrating an example of a process for machine learning enhancement of one or more molecular properties, in accordance with some example embodiments;

FIG. 9B depicts a schematic diagram illustrating different examples of machine learning architectures for implementing a molecule design computation model, in accordance with some example embodiments;

FIG. 9C depicts a graph illustrating a two-dimensional example of matching interventions, in accordance with some example embodiments;

FIG. 10A depicts graphs illustrating the one-shot performance of a molecule design computation model operating on a joint representation that combines the amino acid sequence of an input molecule with the corresponding structural context information, in accordance with some example embodiments;

FIG. 10B depicts a graph illustrating the edit distance between different regions of different input molecules and the matched dataset used for training, in accordance with some example embodiments;

FIG. 11A depicts a schematic diagram illustrating the iterative training of a molecule design computation model using pseudo-matched molecule pairs, in accordance with some example embodiments;

FIG. 11B depicts violin plots illustrating the output distributions of a conditional generative model that underwent iterative training with pseudo-matched (or synthetic) molecule pairs, in accordance with some example embodiments;

FIG. 11C depicts graphs illustrating a comparison of self-training performance for an implicitly guided property optimizer and two variants of a conditional generative model, in accordance with some example embodiments;

FIG. 12 depicts a block diagram illustrating an example of a computing system, in accordance with some example embodiments.

When practical, similar reference numbers denote similar structures, features, or elements.

### DETAILED DESCRIPTION

A molecule may be designed to exhibit multiple desired properties including, in the case of therapeutics, drug-like properties such as binding affinity, specificity, biological activity, developability, and/or the like. In some cases, molecular design may aim to improve one or more properties of an existing molecule. In the case of molecular drug design, a lead molecule, while exhibiting clinically useful pharmacological or biological activities, may have a suboptimal conformation (or three-dimensional structure) that requires modifications to better conform to that of the target. However, the combinatorial space of possible molecular compositions and conformations is far too vast to tackle with conventional molecular design protocols. For example, in the case of small molecules, the molecular space (or chemical space) is estimated to contain 10⁶⁰ possible chemical compounds and scales exponentially with molecule size (e.g., the number of constituent atoms). The size of the combinatorial space is magnitudes larger for larger molecules and biologics. For instance, for a protein molecule containing an N quantity of amino acid residues, approximately 20^{N} possible protein sequences exist if each of the N quantity of amino acid residues is one of the twenty canonical amino acid residues. Each one of the aforementioned 20^{N} possible protein sequences is further capable of adopting an exponential number of conformations (or three-dimensional structures). Even in cases where each one of the N quantity of amino acid residues in a possible protein sequence is limited to assuming one of an M quantity of discrete geometric states (e.g., rotamers), every one of the aforementioned 20^{N} possible protein sequences may still adopt M^{N} possible conformations (or three-dimensional structures).

Existing approaches to molecular design are too resource intensive to support an expansive exploration of the vast combinatorial space of possible molecular compositions and conformations when seeking alternative molecule designs with superior properties than an existing molecule. Lead optimization is an example of a conventional molecular design technique that relies on wet lab measurements, which are scarce in quantity due to the limited availability and exorbitant cost of laboratory resources. Meanwhile, conventional computational techniques are subject to limitations of state-of-the-art computational resources. For example, in silico conformal prediction is time consuming and incapable of determining the conformation (or three-dimensional structure) of more than a few molecules at a time. In other words, given a lead molecule with one or more inadequate properties of interest, conventional molecule design techniques are only able to provide a small number of alternative designs while the prospect that one of these alterative designs would have significantly superior properties than the lead molecule is poor.

Various example embodiments of the present disclosure overcome the limitations of conventional molecular design approaches by leveraging a matched dataset, which contains one or more molecule pairs exhibiting different values for one or more properties of interest, to train a molecule design computation model to recognize features (e.g., compositional features, structural features, and/or the like) that contribute to differences in the values of the one or more properties of interest. For example, in some cases, each molecule pair in the matched dataset may include a first molecule and a second molecule exhibiting different values for at least one property of interest. As described in more details below, in addition to exhibiting the different values for the at least one property of interest, the first molecule and the second molecule may also exhibit at least some similarities, for example, in molecular composition, molecular conformation, and/or the like. When trained based on molecule pairs with counterfactual molecules exhibiting at least some compositional and/or conformational similarities but different values in at least one property of interest, the molecular design computation model may capture the causation (or dependency) between molecular features, such as certain compositional features and/or conformational features, and various properties of interest (e.g., drug-like properties such as binding affinity, specificity, biological activity, developability, and/or the like).

In some example embodiments, two molecules may be identified for inclusion in the matched dataset as a molecule pair based on the two molecules being counterfactuals exhibiting a threshold similarity in molecular composition and/or conformation but different values for at least one property of interest. For example, in some cases, the molecule pair may include a first molecule and a second molecule having different values for at least one property of interest. Examples of properties of interest may include drug-like properties such as affinity, specificity, biological activity, developability, and/or the like. Moreover, as counterfactuals, the first molecule and the second molecule may also exhibit at least some similarities including, for example, similarities in molecular composition and/or conformation, in addition to having different values for at least one property of interest. Accordingly, in some cases, the molecule design engine may identify, based on one or more criteria, the first molecule as a match for the second molecule. In some cases, the one or more criteria may include a proximity metric quantifying a compositional similarity and/or a conformational similarity between the first molecule and the second molecule satisfying one or more thresholds. For instance, where the first molecule and the second molecule are proteins, the proximity metric may quantity the similarity between the respective amino acid sequences and/or the corresponding three-dimensional structures (or conformations). Examples of the proximity metric may include one or more of edit distance, amino acid substitution matrix, a chemical similarity coefficient, a Euclidean distance, atomic coordinates, torsion (or dihedral) angles, and/or the like. Furthermore, in some cases, the one or more criteria may include a difference in the respective values of at least one property between the first molecule and the second molecule satisfying one or more thresholds. In some cases, the molecule design engine may impose the one or more criteria such that the first molecule and the second molecule are exhibit sufficient similarities, for example, in their respective composition and/or conformation, but have different values for at least one property. In some cases, the molecule design computation model may impose the one or more criteria such that the first molecule and the second molecule are the closest (or most similar) counterparts (e.g., exhibiting the highest (or lowest) proximity metric) amongst those available for generating the matched dataset.

In some example embodiments, the molecule design computation model may be trained to improve the properties of an input molecule with implicit guidance such that the output molecule generated therefrom exhibits a superior value for one or more properties of interest, such as affinity, specificity, biological activity, developability, and/or the like. In some cases, this implicit guidance may derive from the molecule design computation model being trained to approximate the gradient of the value of the one or more properties of interest (e.g., a function that predicts the values of the one or more properties present in a molecule). As such, once trained, the molecule design computation model may follow the gradient of the function when generating the output molecule. In some cases, the molecule design computation model may include an encoder and a decoder. In some cases, the encoder and the decoder may be coupled to form an autoencoder. In some cases, the encoder may be trained to generate an embedding of the input molecule while the decoder may be trained to decode the embedding of the input molecule. In some cases, the encoder and the decoder may be trained such that the output molecule that is generated based on the decoded embedding of the input molecule exhibits superior values for at least one property of interest. To achieve this outcome, the training of the molecule design computation model may include adjusting one or more parameters (e.g., weights, biases, and/or the like) of the encoder and the decoder to reduce (or minimize) a reconstruction loss present in the output molecule generated by the decoder decoding the embedding of the input molecule. Accordingly, in this context, the aforementioned reconstruction loss may correspond to the difference between the output molecule and the molecule that is paired with the input molecule in the matched dataset. In some cases, the parameters of the encoder and decoder may be adjusted such to reduce (or minimize) the difference between the output molecule and the molecule paired with the input molecule in the matched dataset. Furthermore, in some cases, the training of the molecule design computation molecule may include imposing a monotonicity constraint to ensure that in instances where one molecule in a molecule pair is greater than the other molecule in the same molecule pair, the reconstruction of the greater molecule is also greater.

In some example embodiments, instead of the aforementioned implicitly guided optimization, the molecule design computation model may be trained to generate molecule designs through sampling with implicit guidance. For example, in some cases, the molecule design computation model may be trained, based on the one or more molecule pairs in the matched dataset, to approximate the data distribution of the molecule pairs. This data distribution may be referred to as a “matched distribution” at least because the high density regions of the data distribution are populated by molecule pairs, with the constituent molecules in each molecule pair exhibiting sufficient similarities in molecular composition and/or conformation but different values for at least one property of interest. As described in more details below, once trained, the molecule design computation model may be applied to sample, for example, over multiple design iterations, from incrementally higher density regions of the matched distribution. In some cases, the training of the molecule design computation model may include training the molecule design computation model to reconstruct, from a noise molecule, one molecule from a molecule pair while being conditioned on the other molecule from the same molecule pair. For instance, in some cases, the molecule design computation model may include a denoiser (or a conditional denoiser) that is trained to denoise the noise molecule to reconstruct the one molecule from each molecule pair with the superior value for at least one property interest while being conditioned on the molecule from the same molecule pair with the inferior value for the at least one property. Conditioning in this context may include training the denoiser (or conditional denoiser) to denoise the noise molecule based on additional information (or context) from the molecule with the inferior value for the at least one property.

In some example embodiments, the two molecules forming each molecule pair in the matched dataset may be matched based on multiple properties such that the molecule design computation model is trained to generate an output molecule having a different value in multiple properties than an input molecule. For example, in the context of antibody design, the molecule design computation model may be trained to generate an output molecule having a superior expression level and binding affinity than the input molecule. Accordingly, in some cases, each molecule pair in the matched dataset for the molecule design computation model may include a first molecule and a second molecule having a superior combination of properties than the first molecule. For instance, in the case of antibody design, the molecule design computation may be trained on based on molecule pairs, each of which including a first molecule and a second molecule having a superior expression level and binding affinity than the first molecule. In some cases, in addition to a proximity metric quantifying a compositional similarity and/or a conformational similarity between the first molecule and the second molecule in each molecule pair, the first molecule and the second molecule may be matched based on a respective multivariate rank indicative of a difference in the combination of properties present in each of the first molecule and the second molecule. The multivariate rank for each of the first molecule and the second molecule may be determined by applying one or more of an expected hypervolume improvement (EHVI), a noisy expected hypervolume improvement (NEHVI), a cumulative distribution function (CDF), a Pareto efficient global optimization (ParEGO), a max-value entropy search method (MESMO), a joint entropy search (JES), and/or the like.

In some example embodiments, once trained, the molecule design computation model may be applied to generate one or more output molecules. In instances where the output molecules are protein molecules, for example, the output of the molecule design computation model may be one or more of the corresponding amino acid sequences. Alternatively, the molecule design computation model may be applied to generate an output that is indicative of one or more modifications, such as compositional modifications and/or conformational modifications, that would change the value of at least one property present in an input molecule. For example, in the case of antibody design, the molecule design computation model may be applied to determine one or more modifications to an input molecule that would change the expression level, the binding affinity, and/or the developability of the input molecule. In some cases, the molecule design computation model may determine the one or more modifications to the input molecule by at least encoding the input molecule before the resulting embedding of the input molecule is decoded. The decoding of the embedding of the input molecule may generate a multinomial distribution of the possible composition and/or conformation of an output molecule exhibiting the one or more modifications. Accordingly, as described in more details below, one or more output molecules may be determined based on the multinomial distribution.

In some example embodiments, one or more output molecules may be determined based on the multinomial distribution determined by the molecule design computation model. As noted, the output of the protein design computation model may include a multinomial distribution of the possible composition and/or conformation of an output molecule exhibiting the one or more modifications that would improve one or more properties of the input molecule. In instances where the input molecule is a protein molecule, this multinomial distribution may include, for every possible position within the corresponding protein sequence, a probability for each possible amino acid residue occupying the position. Accordingly, where a protein sequence has an M quantity of possible positions (e.g., 299 possible positions in an antibody sequence) and there are an N quantity of possible amino acid residues (e.g., 22 canonical amino acid residues), the output of the molecule design computation model may include a M×N vector enumerating every one of the M×N probabilities. In some cases, the molecule design engine may generate one or more output molecules by sampling from the multinomial distribution corresponding to the aforementioned M×N vector. For instance, in some cases, the molecule design engine may generate an output molecule by at least identifying, for one or more of the M quantity of constituent positions, one of the N quantity of possible amino acid residues whose probability of occupying that position satisfies one or more thresholds. Alternatively and/or additionally, the output molecule may be generated by preserving the identities of the amino acid residue occupying one or more of the M quantity of positions in the input molecule. Generating the output molecule in this manner may ensure that the output molecule is sampled from within the neighborhood of the input molecule, meaning that each output molecule should exhibit at least some degree of compositional similarity and/or conformational similarity to the input molecule.

In some example embodiments, the molecule design engine may apply the molecule design computation model in an iterative manner in order to generate, over one or more design iterations, one or more output molecules that exhibit incrementally superior properties than those from previous design iterations. For example, in some cases, the molecule design engine may apply the molecule design computation model to generate a first output molecule having a different value for at least one property than an input molecule. In instances where the first molecule design is determined to satisfy one or more criteria, the molecule design engine may identify the first output molecule as a candidate for further evaluation (e.g., wet lab assessment and/or the like). Alternatively, if the first output molecule fails to satisfy the one or more criteria, the molecule design engine may apply the molecule design computation model again to generate a second output molecule by encoding and decoding the first output molecule. In some cases, the one or more criteria may include a proximity metric quantifying a compositional similarity and/or a conformational similarity between the input molecule and the first output molecule satisfying one or more thresholds. Furthermore, in some cases, the one or more criteria may include a difference in a first value of the at least one property present in the input molecule and a second value of the at least one property present in the first output molecule satisfying one or more thresholds. The one or more criteria may be imposed such that the molecule design engine may continue to apply the molecule design computation model to generate additional output molecules until no further improvements can be made upon the output molecule from a preceding design iteration. This may be the case when the composition and/or conformation of the output molecule is sufficiently similar to that of the input molecule or, alternatively, when the input molecule and the output molecule have sufficiently similar properties.

In some example embodiments, the molecule design computation model may generate an output molecule having a different value in multiple properties than an input molecule by at least encoding the input molecule before the resulting embedding of the input molecule is decoded to generate the output molecule. In some cases, the molecule design computation model may operate on a linear (or one-dimensional) representation of the input molecule in order to generate the output molecule. Where the input molecule and the output molecule are protein molecules, for example, the molecule design computation model may encode an input protein sequence before the resulting embedding is decoded to generate an output protein sequence having a superior value in at least one property than the input protein sequence. Alternatively and/or additionally, the molecule design computation model may operate on a higher dimensional representation of the input molecule in order to generate the output molecule. For example, in some cases, the molecule design computation model may operate on a three-dimensional representation of the input molecule. In some cases, the three-dimensional representation of the input molecule may represent the three-dimensional structure (or conformation) of the input molecule in a variety of different ways. In some cases, the three-dimensional structure (or conformation) of the input molecule be represented as a collection of the atoms (or heavy atoms) forming the input molecule. For instance, in some cases, the three-dimensional representation of the input molecule may be a point cloud representation in which each atom (or heavy atom) in the input molecule is represented as a single point in three-dimensional space. In some cases, instead of the point cloud representation, the three-dimensional representation of the input molecule may be a voxelized representation in which each atom (or heavy atom) in the input molecule is represented as a volume of density across a voxel grid. In some cases, instead of discrete atomic density values across a voxel grid, the three-dimensional representation of the input molecule may a molecular occupancy field, which is a continuous function mapping a location in three-dimensional space (e.g., as identified the coordinates (x,y,z)) to the atomic density at the location. In some cases, the three-dimensional representation of the input molecule may be a latent code (or modulation code) encoding the molecular occupancy field.

In some example embodiments, the molecule design computation model may operate on joint representation of the input molecule that combines, for example, a linear (or one-dimensional) representation of the input molecule with one or more higher dimensional representations of the input molecule. For example, in instances where the input molecule is a protein molecule, the molecule design computation model may operate on a joint representation that combines the amino acid sequence and three-dimensional structure of the input molecule. In some cases, the joint representation of the input molecule may be generated based on the linear (or one-dimensional) representation of the input molecule. For instance, in some cases, a structural encoder may be trained to generate, based on the amino acid sequence of the input molecule, a joint representation of the input molecule that further incorporates the corresponding structural context. In some cases, the structural context may include the position of alpha carbons (Ca), inter-residue distances (e.g., distance between adjacent alpha carbons (Ca)), torsion (or dihedral) angles, and/or the like. In some cases, the molecule design computation model may be trained to reconstruct, from the joint representation of the input molecule, the joint representation of an output molecule with superior values for at least one property of interest. In some cases, the reconstruction of the joint representation may incorporate structural adjacency information to emphasize local structural context during reconstruction. Where the input molecule is a protein molecule, for example, the structural adjacency information may include a data structure (e.g., an adjacency matrix) identifying amino acid residues (e.g., alpha carbons (Ca)) in the input molecule that are within a threshold distance. In some cases, the joint representation of the output molecule may be decoded to determine a linear (or one-dimensional) of the output molecule, such as the amino acid sequence of the output molecule in instances where the output molecule is a protein molecule.

In some example embodiments, the molecule design computation model may generate one or more output molecules with superior values for at least one property of interest by at least determining one or more modifications to an input molecule that improves the values of the at least one property of interest present in the input molecule. In instances where the input molecule is a protein molecule, for example, the one or more modifications may include inserting, deleting, and/or changing an identity (or type) of one or more amino acid residues forming the input molecule. Alternatively, in instances where the input molecule is a chemical compound, the one or more modifications may include the insertion, deleting, and/or substitution of one or more functional groups forming the input molecule. In some cases, the molecule design computation model may also be applied in the context of nucleic acid engineering. For example, in some cases, the molecule design computation model may be applied to modify a nucleic acid. In some cases, the nucleic acid may be an oligonucleotide, such as antisense oligonucleotides (ASOs), small interfering RNAs (siRNAs), microRNAs (miRNAs), aptamers, CpG oligonucleotides, and/or the like. In this context, the one or more modifications determined by the molecule design computation model may include changes to the sugar-phosphate backbone that preserves the nucleotide sequence of the original nucleic acid molecule such that the nucleic acid molecule is still capable of binding to the same target but with superior properties such as toxicity, potency, and/or the like.

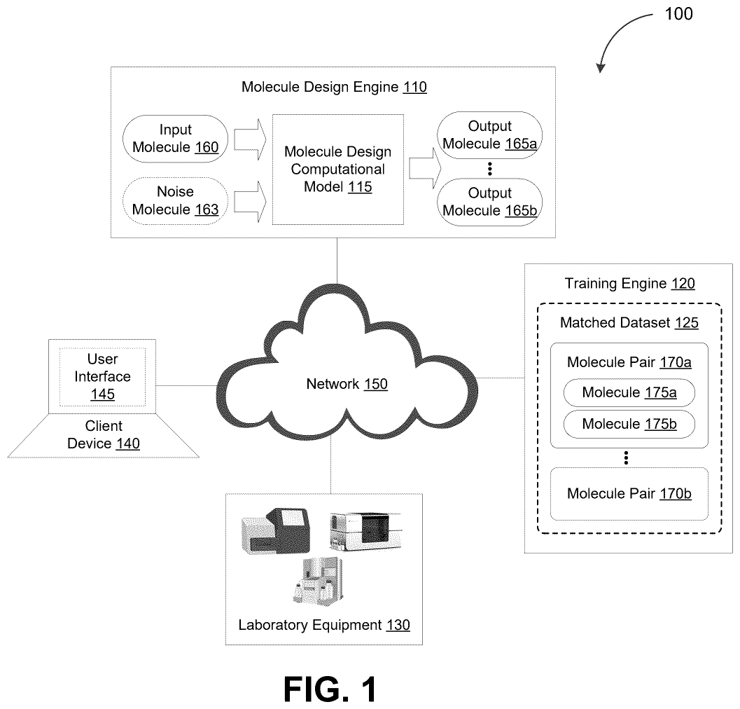

FIG. 1 depicts a system diagram illustrating an example of a molecule design system 100, in accordance with some example embodiments. Referring to FIG. 1 the molecule design system 110 may include a molecule design engine 110, a training engine 120, one or more wet lab equipment 130, and a client device 140. As shown in FIG. 1, the molecule design engine 110, the training engine 120, the one or more laboratory equipment 130, and the client device 140 may be communicatively coupled via a network 150. The one or more laboratory equipment 130 may include any wet lab and dry lab equipment capable of performing in vitro measurements and/or in vivo characterizations. Examples of the one or more laboratory equipment 130 may include sequencers, mass spectrometers, centrifuges, and/or the like. The client device 140 may be a processor-based device including, for example, a workstation, a desktop computer, a laptop computer, a smartphone, a tablet computer, a wearable apparatus, and/or the like. The network 150 may be a wired network and/or a wireless network including, for example, a local area network (LAN), a virtual local area network (VLAN), a wide area network (WAN), a public land mobile network (PLMN), the Internet, and/or the like.

Referring again to FIG. 1, the molecule design engine 110 may apply a molecule design computational model 115 to generate, based at least on an input molecule 160, one or more output molecules 165. For example, the molecule design computation model 115 may be applied to generate a first output molecule 165a based on the input molecule 160 before the molecule design computation model 115 is applied again to generate the second output molecule 165b based on the input molecule 160 or, in some cases, the first output molecule 165a. In some cases, the molecule design computation model 115 may be trained to encode the input molecule 160 before the resulting embedding of the input molecule 160 is decoded to generate the one or more output molecules 165. Alternatively, in some cases, the molecule design computation model 115 may generate the one or more output molecules 165 by denoising a noise molecule 163 while conditioned on the input molecule 160. In some cases, the input molecule 160 may exhibit an inferior value for at least one property of interest. In the context of large molecule therapeutics (or biologics), examples of the at least one property of interest may include expression, binding affinity towards a target molecule, binding specificity, stability, non-immunogenicity, human-ness, absence of self-association (or non-aggregation), lack of chemical liabilities (e.g., aspartate isomerization, oxidation, deamidation), and/or the like. For small molecule drugs (or chemical compounds, examples of the one or more desired properties may include potency (e.g., biochemical potency, cellular potency, in vivo potency), clearance, and permeability. In some cases, the one or more output molecules 165 generated by the molecule design computation model 115 may exhibit one or more modifications, such as modifications to the composition and/or conformation of the input molecule 160, that improve the value of at least one property. As such, in some cases, the one or more output molecules 165 may exhibit superior values for the at least one property when compared to the input molecule 160.

In some cases, the one or more output molecules 165 may be generated based on a multinomial distribution output by the output of the molecule design computation model 115 decoding the embedding of the input molecule 160. In some cases, the multinomial distribution may specify the possible composition and/or conformation of the one or more output molecules 165 exhibiting the one or more modifications that improve the at least one property than the input molecule 160. As described in more details below, in some cases, the one or more output molecules 165 may be generated by sampling from the multinomial distribution. For example, in cases where the input molecule 160 is a protein molecule, the multinomial distribution may specify, for each possible position within the corresponding protein sequence, the probability of the position being occupied by each possible amino acid residue. As such, in some cases, each one of the output sequences 165 may be generated by selecting, for each possible position within the corresponding protein sequence, an amino acid residue whose probability of occupying that position satisfies one or more thresholds. Moreover, in some cases, the molecule design engine 110 generate the one or more output molecules 165 iteratively, with the first output molecule 165a becoming the input molecule 160 from which the molecule design computation model 115 generates the second output molecule 165b during a subsequent design iteration, for example, until one or more criteria are met.

Referring again to FIG. 1, in some example embodiments, the molecule design computation model 115 may be trained based on a matched dataset 120 that includes one or more molecule pairs 170 such as, for example, a first molecule pair 170a, a second molecule pair 170b, and/or the like. In the example shown in FIG. 1, for instance, the first molecule pair 170a in the matched dataset 125 may be generated to include a first molecule 175a and a second molecule 175b, with the two molecules having different values for at least one property. In some cases, the first molecule 175a may have superior (or improved) values for the at least one property than the second molecule 175b.

In the case of antibody design, for example, the first molecule 175a may exhibit a superior expression level, binding affinity, and/or developability than the second molecule 175b. In some cases, the training engine 120 may generate the first molecule pair 170a by identifying, based on one or more criteria, the first molecule 175a as a match for the second molecule 175b. For example, in some cases, the first molecule 175a and the second molecule 175b may be identified as a match if a proximity metric quantifying a compositional similarity and/or a conformational similarity between the first molecule 175a and the second molecule 175b satisfies one or more thresholds. In some cases, the first molecule 175a and the second molecule 175b may be further identified as a match if a difference in a first value of at least one property present in the first molecule 175a and a second value of the at least one property present in the second molecule 175b satisfies one or more thresholds.

Imposing the aforementioned criteria when identifying the first molecule 175a and the second molecule 175b as a match may ensure that the first molecule 175a and the second molecule 175b are counterfactuals, which are counterparts having sufficient similarities in molecular composition and/or molecular conformation but different values for at least one property. As described in more details below, when trained based on the one or more molecule pairs 170, the molecule design computation model 115 may capture the causation (or dependency) between molecular features, such as certain compositional features and/or conformational features, and various molecular properties of interest (e.g., drug-like properties such as affinity, specificity, biological activity, developability, and/or the like).

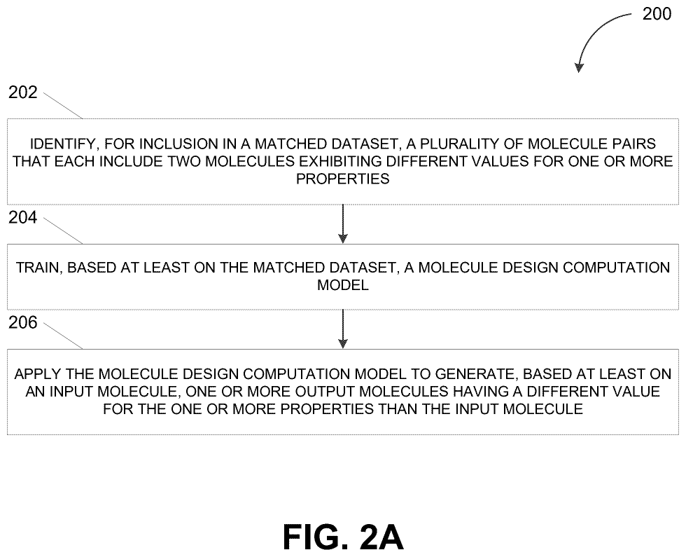

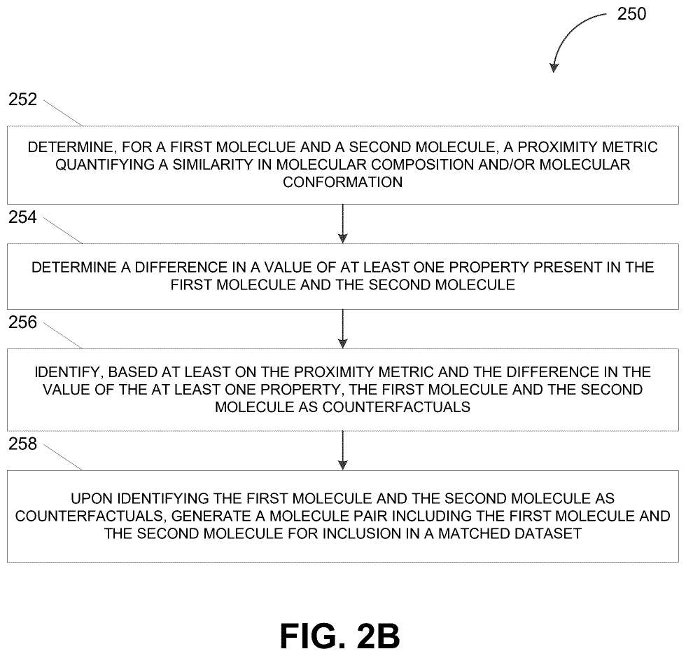

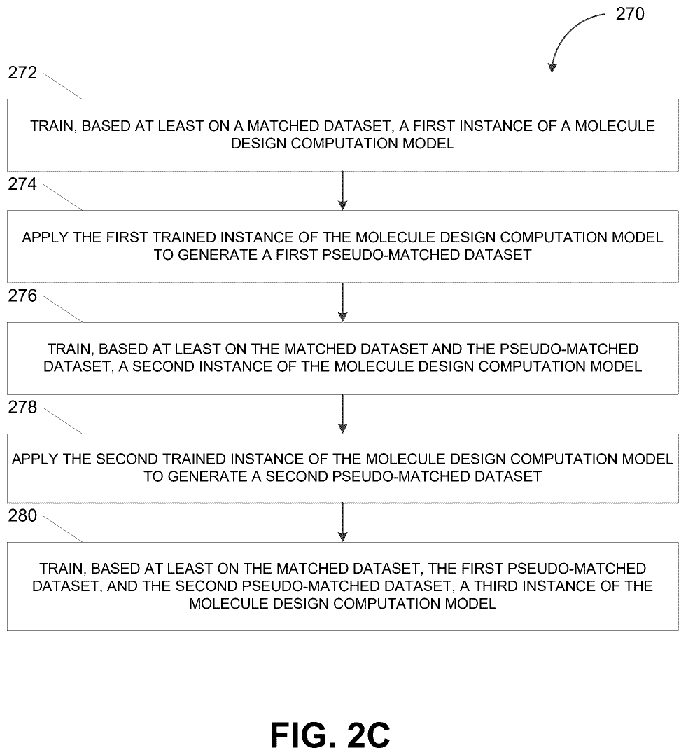

FIG. 2A depicts a flowchart illustrating an example of a process 200 for machine learning enabled enhancement of molecular properties, in accordance with some example embodiments. Referring to FIGS. 1 and 2A, the process 200 may be performed by the training engine 120 to generate, for example, the matched dataset 125, which may be used to train the molecule design computation model 115 to generate the one or more output molecules 165 to exhibit superior values for at least one property than the input molecule 160. Furthermore, in some cases, the process 200 may be performed by the molecule design engine 110, which applies the trained molecule design computation model 115 to improve one or more properties of an input molecule, such as the input molecule 160, by at least determining one or more modifications to the input molecule that would change the values of these properties. As described in more details below, the matched dataset 125 generated by the training engine 120 may include one or more molecule pairs 170, such as the first molecule pair 170a, the second molecule pair 170b, and/or the like. In some cases, the molecule design computation model 115 may be trained based on the matched dataset 125 to recognize features, such as compositional features and/or conformational features, that contribute to differences in the values of one or more molecular properties. Once trained, the molecule design engine 110 may apply the molecule design computation model 115 to generate, based at least on the input molecule 160, for example, the one or more output molecules 165 to have superior values for one or more properties (e.g., drug-like properties) than the input molecule 160.

At 202, a plurality of molecule pairs that each include two molecules exhibiting different values for one or more properties are identified for inclusion in a matched dataset. In some example embodiments, the matched dataset 125 may be generated by identifying one or more molecule pairs, each of which containing two counterfactual molecules. For example, in some cases, a molecule pair 170 may be generated by identifying a first molecule as a match (or counterfactual) for a second molecule. It should be appreciated that the first molecule and the second molecule may be represented in a variety of different ways in order to capture various different compositional and/or conformational features. For instance, in some cases, each of the first molecule and the second molecule may be represented as a real data vector, a point cloud representation, an atomic density field representation, an image pixel representation, a tokenized sequence molecule representations, and/or the like. As described in more details below, possible representations of the first molecule and the second molecule may include a linear (or one-dimensional) representation, for example, of the constituent sequence of amino acid residues. In some cases, possible representations of the first molecule and the second molecule may also include a three-dimensional representation of the constituent atoms (or heavy atoms) in three-dimensional space, such as point cloud, voxel grid, molecular occupancy field, and/or the like. In some cases, possible representations of the first molecule and the second molecule may further include a joint representation that combines, for example, a linear (or one-dimensional) representation of the input molecule with one or more higher dimensional representations of the input molecule. In instances where the first molecule and the second molecule are protein molecules, this joint representation may combine the corresponding amino acid sequence and three-dimensional structure (or conformation).

In some example embodiments, the first molecule may be identified as a match (or counterfactual) for the second molecule if the first molecule and the second molecule exhibit sufficient similarities, for example, in molecular composition and/or molecular conformation, but different values for at least one property of interest (e.g., drug-like properties such as affinity, specificity, biological activity, developability, and/or the like). Accordingly, in some cases, one or more criteria may be imposed when identifying the first molecule as a match (or counterfactual) for the second molecule. For example, in some cases, the one or more criteria may include that a proximity metric quantifying a conformational similarity and/or a compositional similarity between the first molecule and the second molecule satisfying one or more thresholds. Alternatively and/or additionally, the one or more criteria may include that a difference in the values of at least one property present in the first molecule and the second molecule satisfying one or more thresholds. Imposing the aforementioned criteria may ensure that the first molecule and the second molecule identified for inclusion in a molecule pair are counterfactuals, or counterparts having sufficient similarities, for example, in molecular composition and/or molecular conformation, but different values for at least one property of interest. As counterfactual, the difference in the values of the at least one property present in the first molecule and the second molecule may be attributable to compositional and/or conformational differences that exist between the two molecules. As such, as described in more details below, training a molecule design computation model based on a matched dataset that includes molecule pairs formed from counterfactuals such as the first molecule and the second molecule may enable the molecule design computation model to learn the causation (or dependency) between differences in molecular features, such as certain compositional features and/or conformational features, and the corresponding differences in molecular properties.

At 204, a molecule design computation model may be trained based on the matched dataset. In some example embodiments, the molecule design computation model may be trained to generate, based at least on the first molecule in each molecule pair in the matched dataset, a reconstruction corresponding to the second molecule in each molecule pair in the matched dataset. In some cases, the molecule design computation model may be trained, based on the one or more molecule pairs in the matched dataset, to recognize the causation (or dependency) between molecular features, such as certain compositional features and/or conformational features, and various molecular properties of interest. For example, in some cases, the molecule design computation model may include an encoder trained to generate an encoding of the first molecule in each molecular pair and a decoder trained to decode the encoding of the first molecule to recover the second molecule in each molecule pair. In doing so, the molecule design computation model may be trained to approximate the gradient of the value of one or more properties of interest (e.g., a function predicting the value of the one or more properties present in a molecule). In some cases, this gradient may provide implicit guidance when the trained molecule design computation model is applied to generate output molecules exhibiting superior values for one or more properties of interest than the input molecule. In some cases, this paradigm may be tantamount to the optimization of the one or more properties with implicit guidance from the gradient of the value of the one or more properties. Alternatively, in some cases, the molecule design computation model may be trained to perform implicitly guided conditional generation of molecule designs. For instance, in some cases, the molecule design computation model may include a denoiser trained to generate the second molecule in each molecule pair by at least denoising a noise molecule while being conditioned on the first molecule in each molecule pair. In this instance, the molecule design computation model may be trained to approximate a data distribution (or matched distribution) of molecule pairs. Once trained, the trained molecule design computation model may be applied to generate output molecules exhibiting superior values for one or more properties of interest than the input molecule by sampling from the data distribution (or matched distribution).

As noted, in some cases, the molecule design computation model 115 may be trained, based at least on the one or more molecule pairs in the matched dataset, to improve one or more properties of an input molecule by at least generating one or more output molecules with superior values for the one or more properties than the input molecule. For example, in some cases, the molecule design computation model may be trained to determine, based at least on the input molecule, one or more modifications to the input molecule that would improve the one or more properties. As described in more details below, in some cases, the molecule design computation model may output the one or more output molecules, each of which having at least one modification to improve the one or more properties of the input molecule. Alternatively, in some cases, the molecule design computation model may output a multinomial distribution of the possible composition and/or conformation of the one or more output molecules. When the output of the molecule design computation model is a multinomial distribution, the one or more output molecules may be generated by sampling from the multinomial distribution including, in some cases, iteratively over multiple design iterations in which the output molecule that is generated during one design iteration becomes the input molecule that the molecule design computation model operates upon during a subsequent design iteration to generate one or more additional output molecules.

In some example embodiments, the training of the molecule design computation model may include adjusting one or more parameters (e.g., weights, biases, and/or the like) of the molecule design computation model to reduce (or minimize) a loss function. In instances where the molecule design computation model includes an encoder and a decoder (or an autoencoder including the encoder and the decoder), the loss function may include a first loss term corresponding to a reconstruction loss and a second loss term corresponding to a monotonicity constraint. When the molecule design computation model is being trained based on a molecule pair from the matched dataset, the molecule design computation model may encode a first molecule from the molecule pair and decode the resulting embedding of the first molecule in order to generate a reconstruction of the second molecule from the molecule pair. In some cases, the training of the molecule design computation model may include adjusting one or more parameters of the molecule design computation model to reduce (or minimize) a reconstruction loss, which in this case may include a difference between the second molecule in the molecule pair and the reconstruction of that second molecule generated by decoding the embedding of the first molecule. Furthermore, in some cases, the training of the molecule design computation model may include adjusting one or more parameters of the molecule design computation model to conform to a monotonicity constraint such that in cases where the first molecule is greater than the second molecule, the output of the molecule design computation model operating on the first molecule is also greater than the output of the molecule design computation model operating on the second molecule. In some cases, the monotonicity constraint may be enforced in order for the molecule design computation model to preserve, in its output, the order between the molecules in each molecule pair from the matched dataset.

At 206, the molecule design computation model is applied to generate, based at least on an input molecule, one or more output molecules having a different value for the one or more properties than the input molecule. In some example embodiments, once trained, the molecule design computation model may be applied to improve one or more properties of an input molecule. For example, in some cases, the molecule design computation model may be applied to determine one or more modifications to the input molecule that would change the value of one or more properties of interest present in the input molecule. In some cases, the molecule design computation model may output one or more output molecules having the one or more modifications such that the one or more output molecules exhibit superior values for the one or more properties than the input molecule. In the case of antibody design, for example, the molecule design computation model may determine one or more modifications to the input molecule that would increase the expression level, binding affinity, and/or developability of the input molecule.

In some cases, the trained molecule design computation model may include an encoder and a decoder. In some cases, the encoder and the decoder may form an autoencoder. In some cases, to generate an output molecule having the one or more modifications, the encoder may encode the input molecule and the decoder may decode the resulting embedding of the input molecule to generate the output molecule. Alternatively, the molecule design computation model may include a denoiser that generates the output molecule by denoising a noise molecule while being conditioned on the input molecule. In some cases, the one or more output molecules may be generated iteratively, with the molecule design computation model applied to generate the one or more output molecules over multiple, successive design iterations. For example, in some cases, the molecule design computation model may be applied to generate a first output molecule during one design iteration before the molecule design computation model is applied to the first output molecule to generate a second output molecule during a subsequent design iteration. In some cases, instead of the one or more output molecules, the output of the molecule design computation model may include a multinomial distribution of the possible composition and/or conformation of the one or more output molecules exhibiting the one or more modifications. As described in more details below, when the output of the molecule design computation model includes the multinomial distribution, the one or more output molecules 165 may be generated by sampling from the aforementioned multinomial distribution including, in some cases, iteratively over multiple design iterations.

FIG. 2B depicts a flowchart illustrating an example of a process 250 for generating a matched dataset, in accordance with some example embodiments. Referring to FIGS. 1 and 2A-2B, the process 250 may be performed, for example, by the training engine 120 to generate the matched dataset 125 for training the molecule design computation model 115. In some cases, the matched dataset 125 may be generated to include one or more molecule pairs, each of which containing two counterfactual molecules with sufficiently similarities in molecular composition and/or conformation but different values for at least one property of interest. In some cases, the matched dataset 125 may be used to train the molecule design computation model 115 to generate, based at least on the input molecule 160, one or more output molecules 165 exhibiting superior values for at least one property of interest. For example, in some cases, the molecule design computation model 115 may be trained to approximate the gradient of the value of the at least one property (e.g., a function predicting the values of the at least one property of interest present in a molecule). Once trained, this gradient may provide implicit guidance for the molecule design computation model 115 to generate the one or more output molecules 165 to exhibit superior values for the at least one property of interest. Alternatively, the molecule design computation model 115 may be trained to perform implicitly guided conditional generation by at least being trained to approximate a data distribution (or a matched distribution) of the molecule pairs. With implicitly guided conditional generation, the molecule design computation model 115 may sample from the data distribution (or matched distribution) when generating the one or more output molecules 165.

At 252, a proximity metric quantifying a similarity in molecular composition and/or molecular conformation is determined for a first molecule and a second molecule. In some example embodiments, the first molecule and the second molecule may be identified as counterfactuals if the two molecules exhibit sufficient similarities, for example, in molecular composition and/or conformation. Examples of the proximity metric may include one or more of edit distance, amino acid substitution matrix, a chemical similarity coefficient, Euclidean distance, atomic coordinates, torsion (or dihedral) angles, and/or the like. For example, in instances where the first molecule and the second molecule are protein molecules, the proximity metric may be an edit distance or an amino acid substitution matrix quantifying the difference in the corresponding amino acid sequences. In some cases, the proximity metric may also account for similarities in the three-dimensional structure (or conformation) of the first molecule and the second molecule. For instance, where the first molecule and the second molecule are protein molecules, the proximity metric quantifying the similarity between the corresponding three-dimensional structures (or conformations) may include one or more of Euclidean distance, atomic coordinates, and torsion (or dihedral) angles. Where the first molecule and the second molecule are chemical compounds, the proximity metric quantifying the similarity therebetween may include a chemical similarity coefficient. Examples of the chemical similarity coefficient may include Tanimoto (or Jaccard) coefficient, Dice coefficient (or Hodgkin index), cosine coefficient (or Carbo index), Soergel distance, Euclidean distance, Hamming (Manhattan or city-block) distance, and/or the like.

At 254, a difference in a value of at least one property present the first molecule and the second molecule is determined. In some example embodiments, in addition to the exhibiting sufficient similarities in molecular composition and/or conformation as quantified by the proximity metric computed in operation 252, the first molecule and the second molecule may be further identified as counterfactuals based on the first molecule and the second molecule exhibiting different values for at least one property of interest. For example, in some cases, in addition to the first molecule and the second molecule exhibiting sufficient similarities in molecular composition and/or molecular conformation, the two molecules may be further identified as counterfactuals if one molecule exhibits superior values for at least one property of interest than the other molecule. In instances where the first molecule and the second molecule are antibodies, for instance, the first molecule and the second molecule may be identified as counterfactuals if the first molecule and the second molecule exhibit higher expression levels, binding affinity, specificity, thermostability, and/or the like.

At 256, the first molecule and the second molecule are identified as counterfactuals based at least on the proximity metric and the difference in the value of the at least one property. As noted, in some example embodiments, the first molecule and the second molecule may be identified as a match (or counterfactuals) if the first molecule and the second molecule exhibit sufficient similarities in molecular composition and/or molecular conformation but different values for at least one property of interest. Accordingly, in some cases, the first molecule and the second molecule may be identified as a match (or counterfactuals) if the two molecules satisfy one or more criteria. For example, where there is a single property of interest, the one or more criteria may include the proximity metric quantifying the compositional similarity and/or the conformational similarity between the two molecules satisfying one or more thresholds. Furthermore, in the case of a single property of interest, the one or more criteria may include the difference between the value of that single property present in the first molecule and the value of the property present in the second molecule satisfying one or more thresholds.

In some cases, the first molecule and the second molecule may be identified as a match (or counterfactuals) based on a difference in a combination of properties present in the two molecules. For example, in the context of antibody design, the first molecule and the second molecule may be identified as a match (or counterfactuals) if the first molecule exhibits a superior expression level and/or binding affinity than the second molecule. In some cases, a greedy approach may be taken when matching two molecules based on multiple properties. With a greedy approach, the first molecule may be identified as a match for the second molecule if any one property of the first molecule has a superior value than the second molecule 175b. Accordingly, in instances where a greedy approach is applied in the context of antibody design, the first molecule may be identified as a match (or counterfactual) for the second molecule as long as the first molecule exhibits either a superior expression level or superior binding affinity than the second molecule but not necessarily both.

In some example embodiments, instead of a greedy approach to match the first molecule and the second molecule as counterfactuals based on multiple properties, multivariate ranks may be determined instead for each of the first molecule and the second molecule. In some cases, the multivariate rank of each of the first molecule and the second molecule may be determined by applying one or more of an expected hypervolume improvement (EHVI), a noisy expected hypervolume improvement (NEHVI), a cumulative distribution function (CDF), a Pareto efficient global optimization (ParEGO), a max-value entropy search method (MESMO), joint entropy search (JES), and/or the like. Moreover, the multivariate rank of each of the first molecule and the second molecule may rank the corresponding molecules based on a combination of properties. This ranking may be possible even in cases where two or more properties are competitive, meaning that a superior value for one property may not necessarily be accompanied by a superior value for another property. For instance, in the case of antibody design, the multivariate ranks of the first molecule and the second molecule may indicate the extent to which the first molecule improves on the expression level and binding affinity of the second molecule. However, in cases where the first molecule exhibits a superior expression level, its binding affinity may not necessarily be superior than that of the second molecule. The multivariate rank of the first molecule and the multivariate rank of the second molecule may account for the competitive nature of some molecular properties. Where the first molecule exhibits a superior expression level but an inferior binding affinity, the multivariate rank of the first molecule may nevertheless be superior than that of the second molecule depending on the differences in the values of these two properties. Accordingly, the first molecule and the second molecule may be identified as a match if a difference between the multivariate rank of the first molecule and the multivariate rank of the second molecule satisfies one or more thresholds.

At 258, upon identifying the first molecule and the second molecule as counterfactuals, a molecule pair including the first molecule and the second molecule is generated for inclusion in a matched dataset. In some example embodiments, the matched dataset may be generated to include one or more molecule pairs. In some cases, each molecule pair may include two molecules in which one molecule exhibits superior values for at least property of interest than the other molecule in the same molecule pair. As noted, in some cases, the two molecules in each molecule pair may be counterfactuals exhibiting sufficient similarities in molecular composition and/or molecular conformation but different values for the at least one property of interest. Accordingly, in some cases, the matched dataset may approximate the gradient of the value of the at least one property of interest (e.g., a function predicting the value of the at least one property of interest). Moreover, in some cases, the matched dataset may be used to train a molecule design computation model to perform implicitly guided optimization of the at least one property of interest. With implicitly guided optimization, the molecule design computation model may be trained, based on the matched dataset, to approximate the gradient to provide implicit guidance for generating output molecules with superior values for the at least one property of interest than the corresponding input molecules. In some cases, the matched dataset may also correspond to a data distribution (or matched distribution) of molecular pairs in which the constituent molecules are sufficiently similar in molecular composition and/or conformation but one molecule exhibits superior values for at least one property of interest than the other. As described in more details below, in some cases, the matched dataset may also be used to train the molecule design computation model to perform implicitly guided conditional generation. For example, with implicitly guided conditional generation, the molecule design computation model may be trained, based on the matched dataset, to approximate the data distribution (or matched distribution) such that output molecules with superior values for at least one property of interest than the corresponding input molecules may be generated by the molecule design computation model sampling from the data distribution (or matched distribution).

FIG. 2C depicts a flowchart illustrating an example of a process 270 for iteratively training a molecule design computation model on pseudo-matched molecule pairs, in accordance with some example embodiments. Referring to FIGS. 1, 2A, and 2C, the process 270 may be performed, for example, by the training engine 120 to train the molecule design computation model 115 to generate the one or more output molecules 165 to exhibit superior values for one or more properties than the input molecule 160. In some cases, the process 270 may implement operation 204 of the process 200 shown in FIG. 2A. As described in more details below, in some cases, the process 270 may be performed to train the molecule design computation model 115 over multiple successive training cycles. In some cases, the output molecules generated by the molecule design computation model 115 may be used to generate pseudo-matched molecule pairs that augment limited quantity of molecule pairs present in the original matched dataset available to train the molecule design computation model 115. For example, during a successive training cycle, an instance of the molecule design computation model 115 may be trained using pseudo-matched molecule pairs that include output molecules generated by a trained instance of the molecule design computation model 115 from a prior training cycle. The values of the one or more properties of interest present in the output molecules may improve incrementally over successive training cycles, thus enabling the molecule design computation model to be trained on incrementally superior molecules. Accordingly, in some cases, by undergoing iterative training on pseudo-matched molecule pairs, the molecule design computation model may be pushed to generate superior molecules than those in the initial matched dataset available to train the molecule design computation model.

At 272, a first instance of a molecule design computation model is trained based at least on a matched dataset. In some example embodiments, the first instance of the molecule design computation model may be trained based on a matched dataset containing one or more molecule pairs, each of which including two “true” or “real” counterfactual molecules. For example, in some cases, each molecule pair in the matched dataset may include a first sample molecule and a second sample molecule exhibiting different values for at least one property of interest. In some cases, the first sample molecule and the second sample molecule may be counterfactuals, meaning that the two sample molecules may also exhibit at least some compositional and/or conformational similarities. In some cases, the first sample molecule and the second sample molecule may be considered “true” or “real” molecules in the sense that the empirical measurements are available for the values of the one or more properties of interest present in each molecule. In some cases, the first instance of the molecule design computation model may be trained, based on the molecule pairs in the matched dataset, to learn the causation (or dependency) between molecular features, such as certain compositional features and/or conformational features, and the corresponding differences in the one or more properties of interest. For instance, in some cases, the first instance of the molecule design computation model may be trained to approximate the gradient of the one or more properties (e.g., a function predicting the value of the one or more properties) such that the trained molecule design computation model may generate one or more output molecules having superior values for the one or more properties of interest by at least encoding an input molecule and decoding the resulting embedding of the input molecule. Alternatively, the first instance of the molecule design computation model may be trained to approximate the data distribution (or matched distribution) of the molecule pairs in the matched dataset such that the trained molecule design computation model may be applied to generate the one or more output molecules by at least denoising a noise molecule while conditioned on the input molecule.

At 274, the first trained instance of the molecule design computation model is applied to generate a first pseudo-matched dataset. In some example embodiments, the first trained instance of the molecule design computation model (e.g., generated by the training of the first instance of the molecule design computation model in operation 272) may be applied to generate, based on the individual sample molecules from the matched dataset, one or more output molecules. For example, in some cases, the first trained instance of the molecule design computation model may be applied to generate, based at least on a sample molecule from the matched dataset, one or more output molecules exhibiting superior values for one or more properties of interest than the sample molecule. In some cases, a pseudo-matched molecule pair may be generated by pairing the sample molecule with each one of the output molecule generated by the first trained instance of the molecule design computation model operating on that sample molecule. In some cases, an output molecule may be paired with the sample molecule if the two molecules are counterfactuals that exhibit a threshold similarity, for example, in molecular composition and/or conformation, but also a threshold difference in the respective value of the one or more properties of interest. Moreover, in some cases, one or more pseudo-matched molecule pairs may be generated for inclusion in the first pseudo-matched dataset.

At 276, a second instance of the molecule design computation model is trained based at least on the matched dataset and the first pseudo-matched dataset. In some example embodiments, the second instance of the molecule design computation model may share the same architecture as the first instance of the molecule design computation model. Furthermore, in some cases, the second instance of the molecule design computation model may have the same initial parameters (e.g., weights, biases, and/or the like) as the first instance of the molecule design computation model prior to training. In some cases, it may be possible for the second instance of the molecule design computation model to be generated by re-initializing (e.g., to initial values) the parameters (e.g., weights, biases, and/or the like) of the first instance of the molecule design computation model after the first instance of the molecule design computation model has been trained and applied to generate the pseudo-matched dataset. In some cases, the second instance of the molecule design computation model may be trained on a combination of the original matched dataset and the first pseudo-matched dataset generated by pairing sample molecules from the original matched dataset and output molecules generated by the first trained instance of the molecule design computation model based on those sample molecules. For example, in some cases, the second instance of the molecule design computation model may be trained, based on the original matched dataset and the first pseudo-matched dataset, to approximate the gradient of the one or more properties (e.g., a function predicting the value of the one or more properties). The resulting second trained instance of the molecule design computation model may generate one or more output molecules having superior values for the one or more properties of interest by at least encoding an input molecule and decoding the resulting embedding of the input molecule. Alternatively, the second instance of the molecule design computation model may be trained to approximate the data distribution (or matched distribution) of the molecule pairs in the matched and pseudo-matched datasets. Trained in this manner, the resulting second trained instance of the molecule design computation model may be applied to sample one or more output molecules with superior values for the one or more properties of interest from the data distribution (or matched data distribution) by at least denoising a noise molecule while conditioned on the input molecule.

At 278, the second trained instance of the molecule design computation model is applied to generate a second pseudo-matched dataset. In some example embodiments, the second pseudo-matched dataset may be generated by applying the second trained instance of the molecule design computation model (e.g., generated by the training of the first instance of the molecule design computation model in operation 276) to generate, based on the individual sample molecules from the matched dataset, one or more additional output molecules. For example, in some cases, the second trained instance of the molecule design computation model may be applied to generate, based at least on a sample molecule from the matched dataset, one or more output molecules exhibiting superior values for one or more properties of interest than the sample molecule. In some cases, a pseudo-matched molecule pair may be generated by pairing the sample molecule with each one of the output molecule generated by the second trained instance of the molecule design computation model based on that sample molecule. Furthermore, in some cases, one or more pseudo-matched molecule pairs may be generated for inclusion in the second pseudo-matched dataset.

At 280, a third instance of the molecule design computation model is trained based at least on the matched dataset, the first pseudo-matched dataset, and the second pseudo-matched dataset. In some example embodiments, the third instance of the molecule design computation model may share the same architecture as the first and second instances of the molecule design computation model. Furthermore, in some cases, the third instance of the molecule design computation model may have the same initial parameters (e.g., weights, biases, and/or the like) as the first and second instances of the molecule design computation model prior to training. In some cases, it may be possible for the third instance of the molecule design computation model to be generated by re-initializing (e.g., to initial values) the parameters (e.g., weights, biases, and/or the like) of the first instance (or second instance) of the molecule design computation model after the first instance (or second instance) of the molecule design computation model has been trained and applied to generate the pseudo-matched dataset. In some cases, the third instance of the molecule design computation model may be trained on a combination of the original matched dataset, the first pseudo-matched dataset, and the second pseudo-matched dataset. For example, in some cases, the third instance of the molecule design computation model may be trained, based on the original matched dataset, the first pseudo-matched dataset, and the second pseudo-matched dataset, to approximate the gradient of the one or more properties (e.g., a function predicting the value of the one or more properties). The resulting third trained instance of the molecule design computation model may generate one or more output molecules having superior values for the one or more properties of interest by at least encoding an input molecule and decoding the resulting embedding of the input molecule. Alternatively, the third instance of the molecule design computation model may be trained to approximate the data distribution (or matched distribution) of the molecule pairs in the matched and pseudo-matched datasets. Trained in this manner, the resulting third trained instance of the molecule design computation model may be applied to sample one or more output molecules with superior values for the one or more properties of interest from the data distribution (or matched data distribution) by at least denoising a noise molecule while conditioned on the input molecule.

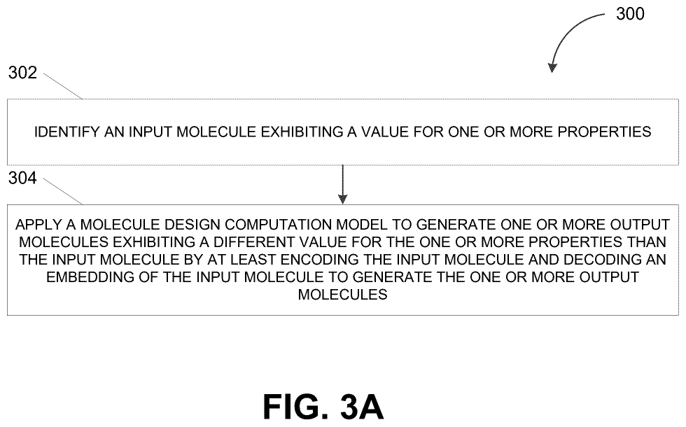

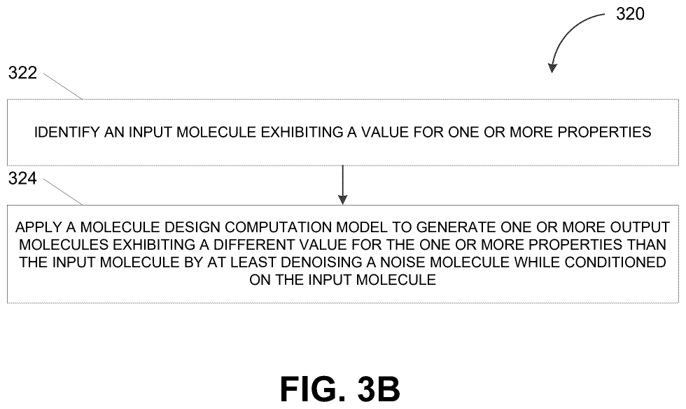

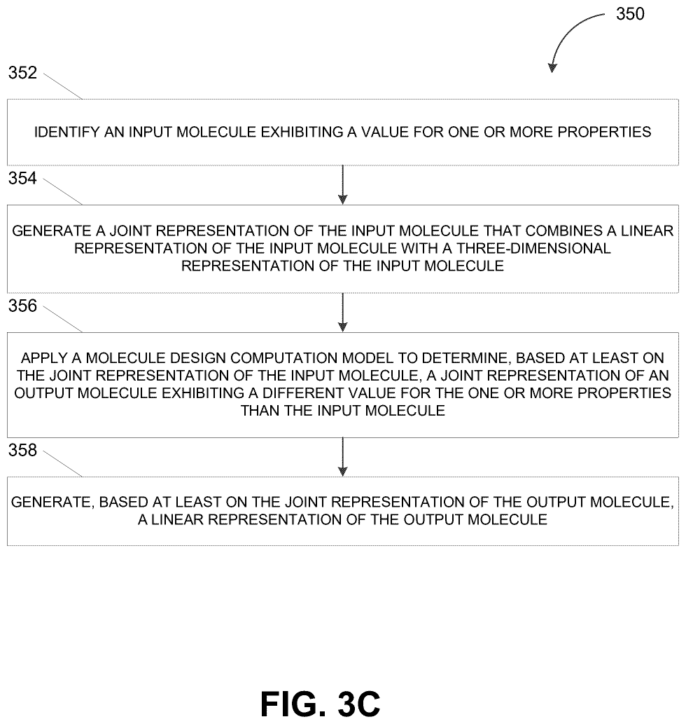

In some example embodiments, a molecule design computation model may be applied to perform implicitly guided optimization of the values of at least one property of interest present in an input molecule such that one or more output molecules generated therefrom exhibits superior values for the at least one property of interest. To further illustrate, FIG. 3A depicts a flowchart illustrating an example of a process 300 for machine learning enabled enhancement of molecular properties, in accordance with some example embodiments. Referring to FIGS. 1, 2A-B, and 3A, the process 300 may be performed, for example, by the molecule design engine 110 to implement at least a portion of operation 206 of the process 200 shown in FIG. 2. In some cases, the molecule design engine 110 may perform the process 300 to improve one or more properties of an input molecule by at least determining one or more modifications to the input molecule that would change the values of these properties. For example, as described in more details below, the molecule design computation model 110 may apply the molecule design computation model 115 to encode the input molecule 160 before the resulting embedding of the input molecule 160 is decoded to generate the one or more output molecules 165. In some cases, the one or more output molecules 165 may exhibit one or more compositional and/or conformational modifications relative to the input molecule 160 that result in the one or more output molecules 165 exhibiting superior values for the one or more properties. In some cases, the molecule design computation model 115 may output a multinomial distribution indicative of the one or more modifications to the input molecule 160 that would improve the values of the one or more properties. For instance, in some cases, the multinomial distribution may specify the possible composition and/or conformation of the one or more output molecules such that the one or more output molecules may be generated by sampling the multinomial distribution.

At 302, an input molecule exhibiting a value for one or more properties is identified. In some example embodiments, the input molecule may exhibit a single or multiple properties that require further improvement. For example, in some cases, the input molecule may be a lead molecule exhibiting at least some desired properties, such as clinically useful pharmacological or biological activities, as well as some undesired properties (e.g., a suboptimal conformation) that require modifications to achieve superior viability as a therapeutic candidate. In the context of therapeutic protein design, the input molecule may be an antibody identified through an animal immunization campaign as having one or more desired properties, such as binding affinity towards an antigen (e.g., a viral antigen, a tumor antigen, and/or the like). However, despite having the one or more desired properties, the input molecule in its original form is unlikely to become a viable protein therapeutic due to the presence of one or more undesired properties, such as insufficient human-ness, poor expression, immunogenicity, in vivo instability, and/or the like. To increase the likelihood that further drug development efforts result in a viable protein therapeutic, the input molecule may therefore undergo lead optimization (LO) in which the composition and/or conformation of the input molecule undergo modifications that would improve the one or more desired properties or, in some cases, reduce (or eliminate) the one or more undesired properties. Accordingly, as described in more details below, one or more properties of the input molecule may be improved by at least applying a molecule design computation model to generate one or more output molecules, each of which having at least one modification relative to the input molecule that changes the values of one or more properties.

At 304, a molecule design computation model is applied to generate one or more output molecules having a different value for the one or more properties than the input molecule by at least encoding the input molecule to generate an embedding of the input molecule and decoding the embedding of the input molecule to generate the one or more output molecules. In some example embodiments, one or more properties of the input molecule may be improved by at least applying a molecule design computation model to determine one or more modifications to the input molecule that improve the one or more properties present in the input molecule. For example, in some cases, the molecule design computation model may generate each output molecule to include at least one modification (e.g., in molecular composition, molecular conformation, and/or the like) that improves the one or more properties of the input molecule. In some cases, the molecule design computation model may operate on a representation of the input molecule 160 captures various compositional and/or conformational features of the input molecule. For instance, in some cases, the molecule design computation model may operate on a voxelized representation, a molecular occupancy field, a real data vector, a point cloud representation, an atomic density field representation, an image pixel representation, and/or a tokenized sequence molecule representation of the input molecule.

In some example embodiments, the molecule design computation model may be trained, based on the molecule pairs (e.g., of counterfactual molecules) in a matched dataset, to approximate the gradient of the value of one or more properties of interest (e.g., a function predicting the values of the one or more properties present in a molecule). In some cases, the generating of the one or more output molecules by the molecule design computation model may be implicitly guided by the aforementioned gradient. For example, in some cases, the molecule design computation model may include an encoder and a decoder. In some cases, the encoder and the decoder may form an autoencoder. In some cases, when applied to generate an output molecule, the encoder may encode the input molecule to an embedding of the input molecule while the decoder may decode the embedding of the input molecule such that the output molecule decoded by the decoder therefrom exhibits a superior value for the one or more properties present in the input molecule. As described in more details below, in some cases, the molecule design computation model may be applied to generate the one or more output molecules over multiple design iterations with implicit guidance from the gradient. For instance, in some cases, the molecule design computation model may generate a first output molecule by at least encoding the input molecule and decoding the resulting embedding of the input molecule. During a subsequent design iteration, the molecule design computation model may generate a second output molecule by at least encoding the first output molecule and decoding the resulting embedding of the first output molecule. In some cases, the molecule design computation model may be applied to generate additional output molecules until one or more criteria are satisfied. In some cases, the one or more criteria may include a lack of adequate improvement being made to the value of the one or more properties, as indicated by the difference between the values of the one or more properties present in output molecules from successive design iterations satisfying (or failing to satisfy) one or more thresholds. As such, in instnaces where the output molecule generated during one design iteration fails to satisfy the one or more criteria, one or more additional design iterations may be performed in which the molecule design computation model is applied to generate additional output molecules.

In some cases, instead of the output of the molecule design computation model being the one or more output molecules, the molecule design computation model may generate an output indicative of one or more modifications to the input molecule that change the values of the one or more properties present in the input molecule. For example, in some cases, the output of the molecule design computation model may include a multinomial distribution of the possible composition and/or conformation of the one or more output molecules exhibiting a superior value for the one or more properties present in the input molecule. In some cases, when the output of the molecule design computation model is the multinomial distribution, one or more output molecules may be generated by sampling from the multinomial distribution. For instance, in the case of protein design, an output molecule may be generated by at least identifying, for each possible position in the corresponding protein sequence, an amino acid residue whose probability of occupying the position satisfies one or more thresholds. In some cases, the identity (or type) of amino acid residue that is identified for each possible position within the output molecule may be the one having the highest probability of occupying the position although it is also possible that for at least some positions within the output molecule, a lower probability amino acid residue may be identified instead. In some cases, it may also be possible to preserve the identity (or type) of one or more amino acid residues present in the input molecule. Doing so may increase the likelihood of the molecule design computation model generating the one or more output molecules to originate from within the neighborhood of the input molecule, meaning that the one or more output molecules may exhibit at least some degree of compositional similarity and/or conformational similarity to the input molecule.

In some example embodiments, a molecule design computation model may be applied to perform implicitly guided conditional generation of one or more output molecules with superior values for at least one property of interest by at least denoising a noise molecule while being conditioned on an input molecule with inferior values for the at least one property of interest. To further illustrate, FIG. 3B depicts a flowchart illustrating another example of a process 320 for machine learning enhancement of one or more molecular properties, in accordance with some example embodiments. Referring to FIGS. 1, 2A-2B, and 3B, the process 320 may be performed, for example, by the molecule design engine 110 to implement at least a portion of operation 206 of the process 200 shown in FIG. 2. In some cases, the molecule design engine 110 may perform the process 320 to improve one or more properties of the input molecule 160 by at least determining one or more modifications to the input molecule 160 that would change the values of these properties. For example, as described in more details below, the molecule design engine 110 may apply the molecule design computation model 115 to generate the one or more output molecule 165 by at least denoising the noise molecule 163 while conditioned on the input molecule 160. In some cases, the one or more output molecules 165 may exhibit one or more compositional and/or conformational modifications relative to the input molecule 160 that result in the one or more output molecules 165 exhibiting superior values for the one or more properties. For instance, in some cases, the molecule design computation model 115 may be trained, based on the molecule pairs 170 in the matched dataset 125, to approximate a data distribution (or matched distribution) of molecule pairs in which one molecule in each molecule pair exhibits a superior value for at least one property than the other molecule in the same molecule pair. In some cases, the molecule design computation model 115 may be applied to generate the one or more output molecules 165 by at least sampling from the data distribution (or matched distribution).

At 322, an input molecule exhibiting a value for one or more properties is identified. In some example embodiments, the input molecule may exhibit a single or multiple properties that require further improvement. For example, in some cases, the input molecule may be a lead molecule exhibiting at least some desired properties, such as clinically useful pharmacological or biological activities, as well as some undesired properties (e.g., a suboptimal conformation) that require modifications to achieve superior viability as a therapeutic candidate. In the context of protein design, the input molecule may be an antibody identified through an animal immunization campaign as having one or more desired properties, such as binding affinity towards an antigen (e.g., a viral antigen, a tumor antigen, and/or the like), but also one or more undesired properties (e.g., insufficient human-ness, poor expression, immunogenicity, in vivo instability, and/or the like) that may renders the input molecule an unsuitable candidate for further drug development in its original form. Accordingly, as described in more details below, one or more properties of the input molecule may be improved by at least applying a molecule design computation model to generate one or more output molecules, each of which having at least one modification relative to the input molecule that changes the values of the one or more properties present in the input molecule.

At 324, a molecule design computation model is applied to generate one or more output molecules having a different value for the one or more properties than the input molecule by at least denoising a noise molecule while conditioned on the input molecule. In some example embodiments, the molecule design computation model may be trained, based on the molecule pairs (e.g., of counterfactual molecules) in a matched dataset, to approximate a data distribution (or matched distribution) of molecule pairs in which one molecule in each molecule pair exhibits a superior value for one or more properties than the other molecule in the same molecule pair. In some cases, the molecule design computation model may generate each output molecule by sampling from the data distribution (or matched distribution). For example, in some cases, the molecule design computation model may generate the one or more output molecules over multiple successive design iterations. In some cases, molecule pairs in which one molecule exhibits a superior value for one or more properties than the other molecule in the same molecule pair may populate the higher density regions of the data distribution (or matched distribution). As such, during each successive design iteration, the molecule design computation model may further modify the input molecule (or the output molecule from a preceding design iteration), thereby sampling one or more additional output molecules from a higher density region of the data distribution (or matched distribution) than the preceding design iteration. For instance, in some cases, the molecule design computation model may sample, from the data distribution (or matched distribution), one or more molecule pairs. By being conditioned on the input molecule, which exhibits an inferior value for the one or more properties, each molecule pair sampled from the data distribution (or matched distribution) may include an output molecule exhibiting a superior value for the one or more properties compared to the input molecule.

In some example embodiments, a molecule design computation model may be applied to perform implicitly guided optimization and/or generation by operating on a joint representation of an input molecule. To further illustrate, FIG. 3C depicts a flowchart illustrating another example of a process 350 for machine learning enhancement of one or more molecular properties, in accordance with some example embodiments. Referring to FIGS. 1, 2A-2B, and 3C, the process 350 may be performed, for example, by the molecule design engine 110 to implement at least a portion of operation 206 of the process 200 shown in FIG. 2. In some cases, the molecule design engine 110 may perform the process 350 to improve one or more properties of the input molecule 160 by at least determining one or more modifications to the input molecule 160 that would change the values of these properties. In some cases, instead of operating on a linear representation of the input molecule 160, such as the amino acid sequence of the input molecule 160 in instances where the input molecule 160 is a protein molecule, the molecule design computation model 115 may be applied to operate on a joint representation of the input molecule 160 that combines the linear representation of the input molecule 160 with a higher dimensional representation, such as a three-dimensional representation of the input molecule 160. As described in more details below, the molecule design engine 110 may apply the molecule design computation model 115 to generate the one or more output molecule 165 by at least operating on the joint representation of the input molecule 160. For example, in some cases, the molecule design computation model 115 may encode the joint representation of the input molecule 160 before decoding the resulting embedding to generate a joint representation of the one or more output molecules 165. In some cases, a linear representation of each of the one or more output molecules 165 may be recovered by further decoding the corresponding joint representation.

At 352, an input molecule exhibiting a value for one or more properties is identified. In some example embodiments, the input molecule may exhibit a single or multiple properties that require further improvement. For example, in some cases, the input molecule may be a lead molecule (e.g., an antibody identified through an animal immunization campaign) exhibiting at least some desired properties, such as clinically useful pharmacological or biological activities, as well as some undesired properties (e.g., a suboptimal conformation) that require modifications to achieve superior viability as a therapeutic candidate. Accordingly, in some cases, one or more properties of the input molecule may be improved by at least applying a molecule design computation model to generate one or more output molecules, each of which having at least one modification relative to the input molecule that changes the values of the one or more properties present in the input molecule. As described in more details below, the molecule design computation model may operate on a joint representation of the input molecule that combines multiple representations of the input molecule such as a linear (or one-dimensional) representation of the input molecule with a corresponding two-dimensional or three-dimensional representation. For instance, where the input molecule is a protein molecule (e.g., an antibody identified through an animal immunization campaign), the joint representation of the input molecule may combine the amino acid sequence of the input molecule with a three-dimensional representation of the input molecule that specifies at least a portion of the three-dimensional structure (or conformation) adopted by the folding of the amino acid sequence.

At 354, a joint representation of the input molecule that combines a linear representation of the input molecule with a three-dimensional representation of the input molecule is generated. In some example embodiments, the joint representation of the input molecule may combine multiple representations of the input molecule such as, for example, a linear (or one-dimensional) representation with a two- or three-dimensional representation of the input molecule. As noted, where the input molecule is a protein molecule, the joint representation of the input may combine the amino acid sequence of the input molecule with a three-dimensional representation of the input molecule that specifies at least a portion of the three-dimensional structure (or conformation) adopted by the folding of the amino acid sequence. For example, in some cases, the joint representation of the input molecule may be a per-residue embedding that includes, for each amino acid residue present in the input molecule, structural context information such as one or more amino acid residues that are adjacent in three-dimensional space. Alternatively, where the input molecule is a chemical compound, the joint representation of the input molecule may combine a linear (or one-dimensional) representation of the input molecule (e.g., a simplified molecular-input line-entry system (SMILES) string) with a two-dimensional representation (e.g., a molecular graph) and/or a three-dimensional representation (e.g., point cloud, atomic density field) of the input molecule.

In some cases, the joint representation of the input molecule may incorporate structural context information that are scarce (or nonexistent) in the linear (or one-dimensional) representation of the input molecule. In some cases, the joint representation of the input molecule may be generated by a structural encoder trained to incorporate, based on the linear (or one-dimensional) representation of the input molecule, the corresponding two-dimensional or three-dimensional structural context information. For example, in some cases, the structural encoder may be trained to determine, based at least on the amino acid sequence of the input molecule, the corresponding structural context information. In some cases, the structural encoder may be trained based on different dimensional representations of the same sample molecules such that the structural encoder is capable of inferring two- and/or three-dimensional structural context based on the linear (or one-dimensional) representation of the input molecule. Where the input molecule is a protein molecule, for example, the structural context information may identify adjacent amino acid residues in three-dimensional space. For instance, in some cases, the structural context information may include a data structure (e.g., an adjacency matrix) identifying adjacent amino acid residues in three-dimensional space. In this context, two amino acid residues may be identified as adjacent (or nonadjacent) amino acid residues if the distance between the two amino acid residues (or the constituent alpha carbons (C_{α})) in three-dimensional space satisfies one or more thresholds (e.g., 8 to 10 angstroms). In some cases, the rows and the columns of the adjacency matrix may correspond to the individual amino acid residues that are present in the input molecule. In this example, the input molecule may include n amino acid residues a₁, . . . , aₙ. Except for the entries along the diagonal of the adjacency matrix, which corresponds to the same amino acid residue, each entry in the adjacency matrix corresponds to a pair of amino acid residues in the input molecule. In some cases, the adjacency matrix may be binary, meaning that each entry includes a first value (e.g., “1”) when the corresponding pair of amino acid residues are located within a threshold distance in three-dimensional space and a second value (e.g., “0”) when the corresponding pair of amino acid residues are not located within the threshold distance in three-dimensional space. In some cases, instead of binary values, it should be appreciated that it is also possible for the adjacency matrix may be occupied by values from a more granular scale for classifying the distance between amino acid residues in the input molecule.

It should be appreciated that neighboring amino acid residues within the amino acid sequence of the input molecule may not necessarily be proximately located in three-dimensional space. Accordingly, the incorporation of structural context information, which identifies adjacent amino acid residues in three-dimensional space, may provide additional information during the implicitly guided optimization and/or generative process in which a molecule design computation model is applied to operate on the joint representation of the input molecule and generate one or more output molecules with superior values for at least one property of interest.

At 356, a molecule design computation model is applied to determined, based at least on the joint representation of the input molecule, a joint representation of an output molecule exhibiting a different value for the one or more properties than the input molecule. In some example embodiments, when the molecule design computation model is trained to perform property optimization with implicit guidance (e.g., from a matched dataset), the molecule design computation model may determine the joint representation of the output molecule by at least further encoding the joint representation of the input molecule before the resulting embedding is decoded to generate the joint representation of the output molecule. Alternatively, in some cases, the molecule design computation model may be trained to perform conditional generation with implicit guidance (e.g., from the matched dataset), in which case the molecule design computation model may generate the joint representation of the output molecule by at least denoising a noise molecule while conditioned on the joint representation of the input molecule.

As noted, in some cases, the joint representation of the input molecule may incorporate structural context information, such as amino acid residues that are adjacent in three-dimensional space, that is unavailable in a linear (or one-dimensional) representation of the input molecule. Accordingly, in some cases, the incorporation of structural context information may improve the performance of the molecule design computation model or, in some cases, extend the capabilities of the molecule design computation model to zero- or one-shot optimization and/or generation. For example, in some cases, the molecule design computation model may be applied to perform “one-shot” optimization and/or generation by operating on the joint representation of an out-of-distribution (OOD) input molecule. In the context of protein design, the input molecule may be considered “out-of-distribution” if the amino acid sequence of the input molecule is outside of the distribution of the amino acid sequences of the sample molecules forming the matched dataset used to train the molecule design computation model. Nevertheless, despite the amino acid sequence of the input molecule being out-of-distribution (OOD), it may still be possible for the three-dimensional structure (or conformation) adopted by this amino acid sequence to exhibit similar biophysical patterns as the sample molecules in the matched dataset. Thus, when the molecule design computation model operates on the joint representation of the input molecule, it may leverage the structural context information included in the joint representation to generate output molecules with superior values for at least one property of interest despite the amino acid sequence of the input molecule being out-of-distribution.

At 358, a linear representation of the output molecule is generated based at least on the joint representation of the output molecule. In some example embodiments, a structural decoder may be applied to recover, from the joint representation of the output molecule, the linear representation of the output molecule. Where the output molecule is a protein molecule, for example, the linear representation of the output molecule recovered from its joint representation may include an amino acid sequence of the output molecule. Alternatively, where the output molecule is a chemical compound, the structural decoder may be applied to recover the simplified molecular-input line-entry system (SMILES) string, an international chemical identifier (InChI), a SELF-referencing embedded string (SELFIES), and/or the like.

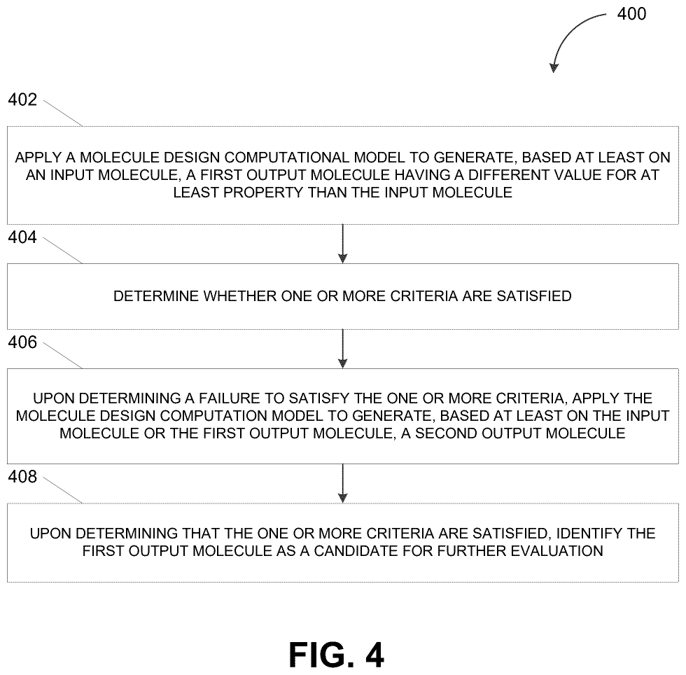

In some example embodiments, a molecule design computation model may be applied to generate output molecules with superior values for at least one property of interest in an iterative manner. For example in some cases, the molecule design computation model may be applied to generate, based at least on an input molecule, a first output molecule having a superior value for at least one property of interest than the input molecule. During a subsequent design iteration, the molecule design computation model may be applied to generate, based at least on the first output molecule, a second output molecule having a superior value for the at least one property of interest than the first output molecule. To further illustrate, FIG. 4 depicts a flowchart illustrating an example of a process 400 for iterative enhancement of molecular properties, in accordance with some example embodiments. Referring to FIGS. 1-4, the process 400 may be performed by the molecule design engine 110 in order to generate, iteratively over multiple design iterations, the one or more output molecules 165 to exhibit incrementally superior, improved values of properties until one or more criteria are met. As described in more details below, in cases where the first output molecule 165a generated during one design iteration fails to satisfy the one or more criteria, the first output molecule 165a may become the input molecule 160 during a subsequent design iteration with the molecule design engine 110 applying the molecule design computation model 115 to generate the second output molecule 165b based on the first output molecule 165a.

At 402, a molecule design computation model is applied to generate, based at least on an input molecule, a first output molecule having a different value for at least one property than the input molecule. In some example embodiments, the molecule design computation model may be applied to improve one or more properties of the input molecule by at least generating an output molecule having one or more modifications (e.g., compositional modifications, conformational modifications, and/or the like) relative to the input molecule that change the value of the one or more properties present in the input molecule. In some cases, the molecule design computation model may be trained based on a matched dataset containing multiple molecule pairs, each of which including two counterfactual molecules exhibiting sufficient similarities in molecular composition and/or conformation but different values for at least one property.

In some cases, the molecule design computation model may be trained based on the matched dataset to recognize features (e.g., compositional features, structural features, and/or the like) that contribute to differences in the values of the one or more properties. For example, in some case, the molecule design computation model may be trained to approximate the gradient of the value of the one or more properties (e.g., a function predicting the values of the one or more properties) such that the gradient may provide implicit guidance when the molecule design computation model is applied to optimize at least one property of the input molecule. The molecule design computation model in this case may generate an output molecule by at least encoding the input molecule and decoding the resulting embedding of the input molecule. Alternatively, the molecule design computation model may be trained based on the matched dataset to approximate a data distribution (or matched distribution) of the molecule pairs from the matched dataset such that the molecule design computation model is able to perform implicitly guided conditional generation. In this instance, the molecule design computation model may generate one or more output molecules by at least sampling from the data distribution (or matched distribution), for example, by denoising a noise molecule while being conditioned on the input molecule.

In some cases, the output of the molecule design computation model may include the one or more output molecules. For example, in some cases, the output of the molecule design computation model may include a linear (or one-dimensional) representation, a two-dimensional representation, or a three-dimensional representation of each output molecule. Alternatively, in some cases, the output of the molecule design computation model may include a joint representation of each output molecule that combines a linear (or one-dimensional) representation of the output molecule with a higher dimensional representation. As noted, in some cases, it may also be possible for the molecule design computation model to output a multinomial distribution of the possible composition and/or conformation of the one or more output molecules. Accordingly, in some cases, each output molecule may be further generated by at least sampling from the multinomial distribution. For instance, in the case of protein design, an output molecule may be generated by at least identifying, for each possible position within a corresponding protein sequence, an amino acid residue whose probability of occupying the position according to the multinomial distribution satisfies one or more thresholds.

At 404, whether one or more criteria are satisfied may be determined. In some example embodiments, the molecule design computation model may be applied to generate multiple output molecules over multiple successive design iterations until one or more criteria are satisfied. In some cases, the one or more criteria may be imposed in order to determine whether additional modifications to the input molecule or the output molecules from a preceding design iteration are necessary to further improve one or more properties of the input molecule. For example, in some cases, the one or more criteria may include a proximity metric quantifying the similarity, such as compositional similarity and/or conformational similarity, between each output molecule from a current design iteration and the input molecule satisfying one or more thresholds. Alternatively and/or additionally, the one or more criteria may include a proximity metric quantifying the similarity, such as compositional similarity and/or conformation similarity, between each output molecule from the current design iteration and one or more output molecules from a preceding design iteration satisfying one or more thresholds. In some cases, satisfying the one or more criteria may constitute an indication that no additional modifications can be made to the input molecule and/or the output molecule from the current design iteration to further improve the one or more properties.

In some cases, the one or more criteria may also include a difference between the values of the one or more properties present in the output molecule from the current design iteration and the values of the one or more properties present in the input molecule satisfying one or more thresholds. In some cases, it may also be possible for the one or more criteria to further include a difference between the values of the one or more properties present in the output molecule from the current design iteration and the values of the one or more properties from a preceding design iteration satisfying one or more thresholds. Satisfying either or both of the aforementioned criteria may indicate that the one or more properties cannot be further improved even with additional modifications.

At 406, upon determining a failure to satisfy the one or more criteria, apply the molecule design computation model 115 to generate, based at least on the input molecule or the first output molecule, a second output molecule. As noted, in some example embodiments, the one or more criteria may include sufficient similarities in molecular composition and/or conformation. For example, when the first output molecule and the input molecule (or one or more output molecules from a preceding design iteration) fail to exhibit sufficient compositional similarity and/or conformational similarity, the input molecule or the first output molecule may still undergo additional modifications to further improve the one or more properties present therein. In some cases, the one or more criteria may also include a sufficient difference in the values of the one or more properties. For instance, it may be possible for the input molecule or the first output molecule to undergo additional modifications to further improve the values of the one or more properties if the difference between the value of the one or more properties present in the first output molecule and the value of the one or more properties present in the input molecule (or one or more output molecules from a preceding design iteration) fails to satisfy one or more thresholds.

In some cases, when the one or more criteria are not satisfies, the molecule design computation model may be applied to generate one or more additional output molecules including, for example, by further modifying the input molecule or one or more output molecules from a preceding design iterations. For example, in some cases, the molecule design computation model may be applied to generate, based at least on the input molecule or the first output molecule, a second output molecule. In some cases, the input molecule may undergo iterative modifications meaning that the first output molecule may become the input molecule during a subsequent design iteration in which the molecule design computation model is applied to generate, based on the first output molecule, the second output molecule.

At 408, upon determining that the one or more criteria are satisfied, identify the first output molecule as a candidate for further evaluation. In some example embodiments, satisfying the one or more criteria may indicate that no additional modifications may be made to the first output molecule to further improve the one or more properties present therein. For example, in instances where the proximity metric quantifying the similarity between the first output molecule and the input molecule (or one or more output molecules from a preceding design iterations) satisfies one or more thresholds, the molecules may be sufficiently similar (e.g., in composition, conformation, and/or the like) to preclude additional modifications to from further improving on the one or more properties present therein. Alternatively and/or additionally, the difference between the value of the one or more properties present in the first output molecule and the value of the one or more properties present in the input molecule (or one or more output molecules from the preceding design iteration) may also satisfy one or more thresholds, meaning that the one or more properties of the molecules cannot be further improved even with additional modifications. In instances where no additional modifications may be made to the first output molecule (or the input molecule) to further improve the one or more properties present therein, the first output molecule may be identified as a candidate for further evaluation, including wet lab assessment such as in vitro measurements, in vivo characterization, and/or the like.

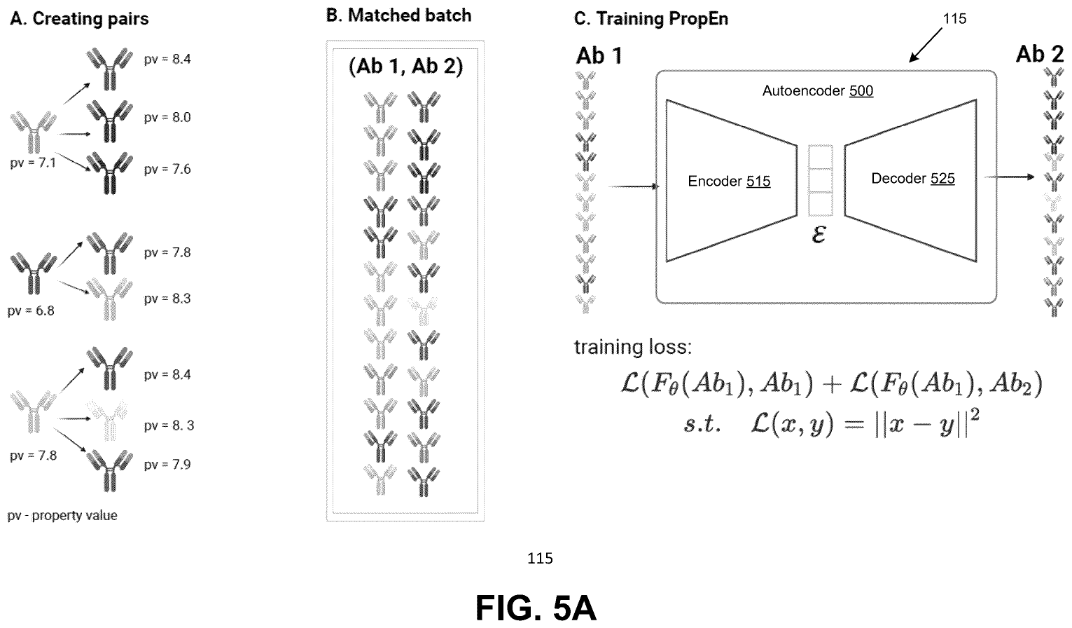

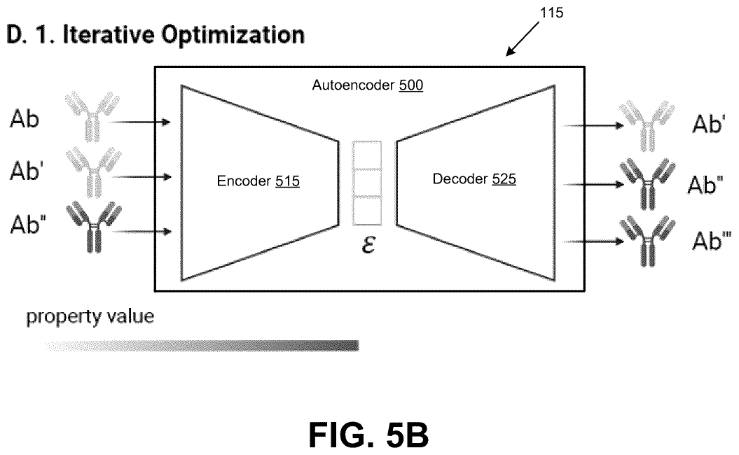

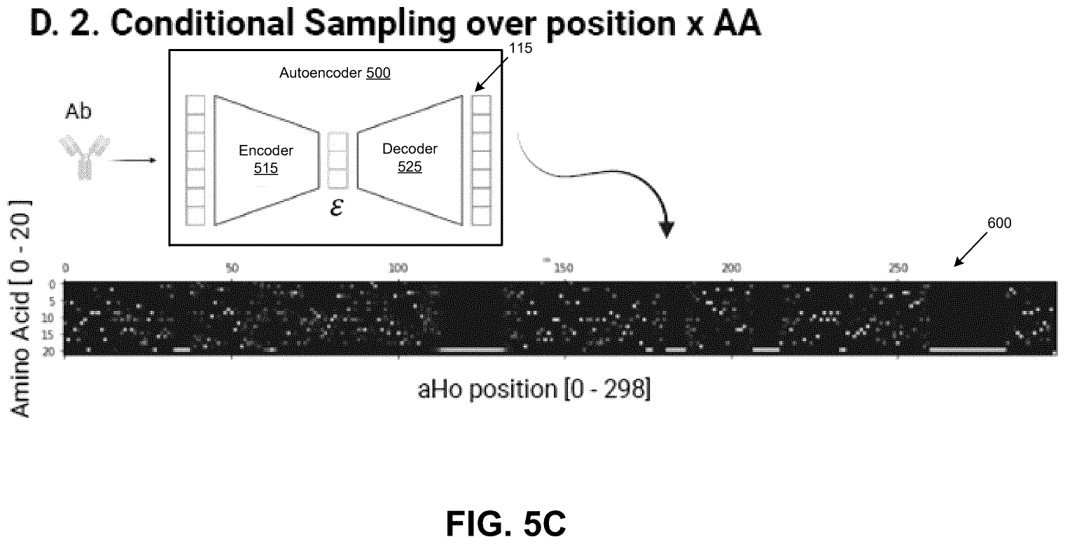

FIG. 5A depicts a schematic diagram illustrating an example of a process for training a molecular design computation model based on one or more molecule pairs in a matched dataset, in accordance with some example embodiments. As noted, in some example embodiments, the training engine 120 may generate the matched dataset 125 to include the one or more molecule pairs 170, each of which having two molecules exhibiting two different values for at least one property (e.g., binding affinity, binding specificity, hydrophobicity, size of electrical charge patches, angle delta, angle length, immunogenicity, presence of liability motifs, and/or the like). For example, the first molecule pair 170a may include the first molecule 175a and the second molecule 175b having a different value for at least one property than the first molecule 175a. To further illustrate, FIG. 5A shows pairs of antibodies being matched based on differences in property value. In the example shown in FIG. 5A, the training engine 120 may generate a matched batch of antibody pairs (Ab₁, Ab₂) in which a first antibody Ab₁ having a first property value is matched with a second antibody Ab₂ having a second property value. In some cases, the training engine 120 may impose one or more criteria when matching the first antibody Ab₁ and the second antibody Ab₂ in each antibody pair in the matched batch. For instance, the one or more criteria may include the difference between the first property value of the first antibody Ab₁ and the second property value of the second antibody Ab₂ satisfying one or more thresholds. Alternatively and/or additionally, the one or more criteria may include a proximity metric quantifying the similarity, such as the compositional similarity and/or conformational similarity, between the first antibody Ab₁ and the second antibody Ab₂ in each antibody pair satisfying one or more thresholds. By imposing these criteria when generating the antibody pairs in the matched batch, the training engine 120 may ensure that the first antibody Ab₁ and the second antibody Ab₂ are counterfactuals exhibiting at least some similarity (e.g., in molecular composition, molecular conformation, and/or the like) but different property values. In some cases, the difference in the property values of the first antibody Ab₁ and the second antibody Ab₂ may be attributable to the differences therebetween (e.g., in molecular composition, molecular conformation, and/or the like). As described in more details below, in some cases, the molecule design computation model 115 may be trained, based on the antibody pairs (Ab₁, Ab₂) in the matched batch, to learn the causation (or dependency) between differences in molecular features, such as certain compositional features and/or conformational features, and the corresponding differences in molecular properties.

Referring again to FIG. 5A, in some cases, the molecule design computation model 115 may be trained based on the antibody pairs (Ab₁, Ab₂) in the matched batch. In the example shown in FIG. 5A, the molecule design computation model 115 may include an autoencoder 500. The autoencoder 500 may include an encoder 515 coupled with a decoder 525. In some cases, each of the encoder 515 and the decoder 525 may be implemented with one or more neural networks, although any other machine learning architectures (or combinations thereof) are also contemplated. As shown in FIG. 5A, in some cases, for each antibody pair (Ab₁, Ab₂), the encoder 515 may encode the first antibody Ab₁ to generate an embedding ε thereof while the decoder 525 may then decode the embedding ε. However, instead of recovering the first antibody Ab₁ by decoding the embedding ε of the first antibody Ab₁, the autoencoder 500 may be trained to generate a reconstruction of the second antibody Ab₂ by the decoding of the embedding ε. In other words, the training of the molecule design computation model 115 may include adjusting one or more parameters (e.g., weights, biases, and/or the like) of the autoencoder 500 such that the encoder 515 generates the embedding ε of the first antibody Ab₁ to enable the decoder 525 to recover the second antibody Ab₂ therefrom.

In some example embodiments, once the molecule design computation model 115 is trained, the molecule design computation model 115 may be applied to improve one or more properties of the input molecule 160. In some cases, the molecule design engine 110 may apply the molecule design computation model 115 to improve one or more properties of the input molecule 160 in an iterative manner. That is, in some cases, once the molecule design engine 110 may applies the molecule design computation model 115 to generate the first output molecule 165a based on the input molecule 160, the molecule design engine 110 may continue applying the molecule design computation model 115 to generate the second output molecule 165b based on the first output molecule 165a if the first output molecule 165a fails to satisfy one or more criteria.

To further illustrate, FIG. 5B depicts a schematic diagram illustrating an example of a process for iterative optimization of molecular properties, in accordance with some example embodiments. As shown in FIG. 5B, the molecule design engine 110 may apply the molecule design computation model 115 (e.g., the autoencoder 500) to generate, based on an input antibody Ab, a first output antibody Ab′. For instance, in the example shown in FIG. 5B, the encoder 515 may encode the input antibody Ab to generate an embedding ε of the input antibody Ab before the decoder 525 decodes the embedding ε of the input antibody Ab to generate the first output antibody Ab′. In some cases, the first output antibody Ab′ may exhibit one or more modifications (e.g., compositional modifications, conformational modifications, and/or the like) that improve the property value of the input antibody Ab. In cases where the first output antibody Ab′ fails to satisfy one or more criteria (e.g., proximity metric, property value, and/or the like) despite the one or more modifications, the first output antibody Ab′ may become the input for a subsequent design iteration. As shown in FIG. 5B, the molecule design engine 110 may apply the molecule design computation model 115 to generate, based at least on the first output antibody Ab′, a second output antibody Ab″ to include one or more additional modifications to further improve the property value of the first output antibody Ab′. In the event the second output antibody Ab″ still fails to satisfy the one or more criteria, the molecule design engine 110 may apply the molecule design computation model 115 again to generate, based at least on the second output antibody Ab″, a third output antibody Ab′″. This iterative process may continue, with one or more additional design iterations, until the one or more criteria are met. For instance, in some cases, the molecule design engine 110 may terminate the iterative process and identify the third output antibody Ab′″ as a candidate for further evaluation (e.g., wet lab assessment such as in vitro measurements, in vivo characterization, and/or the like) if the molecule design engine 110 determines, based at least on the one or more criteria, that no additional modifications may be made to the third output antibody Ab′″ to further improve its properties.

In some example embodiments, the output of the molecule design computation model 115 may include a multinomial distribution of the possible composition and/or conformation of the one or more output molecules 165 generated based on the input molecule 160. FIG. 5C depicts an example of a multinomial distribution 550, in accordance with some example embodiments. In the example shown in FIG. 5C, the encoder 615 of the molecule design computation model 115 may encode the input antibody Ab to generate the embedding ε of the input antibody Ab before the decoder 525 decodes the embedding ε of the input antibody Ab to generate the multinomial distribution 550. The example of the multinomial distribution 550 shown in FIG. 5C is generated based on a fixed-length representation (e.g., AHo representation) of the input antibody Ab that has 298 possible positions. In some cases, the absence of an actual amino acid residue in a particular position may be indicated by the position being populated by a gap character. As such, the example of the multinomial distribution 550 shown in FIG. 5C includes, for each one of the 298 possible positions, the probability of the position being occupied by each one of the 20 possible amino acid residues as well as the probability of the position being vacant.

In some example embodiments, the molecule design engine 110 may generate different output antibodies, such as the first output antibody Ab′, the second output antibody Ab″, and the third output antibody Ab′″, by sampling from the multinomial distribution 550. For example, in some cases, the molecule design engine 110 may select, for each position in the first output antibody Ab′, an amino acid residue or a gap character whose probability of being in the position satisfies one or more thresholds. Furthermore, in some cases, the molecule design engine 110 preserve the composition of at least some portions of the input antibody Ab, meaning that the molecule design engine 110 may keep the same amino acid residues or gap characters in one or more positions in the first output antibody Ab′ as in the input antibody Ab. Sampling from the multinomial distribution 550 in this manner may result in the first output antibody Ab′ being sampled from the neighborhood of the input antibody Ab. That is, in some cases, the molecule design engine 110 may sample from the multinomial distribution 550 to generate the first output antibody Ab′ to exhibit at least some degree of similarity (e.g., in composition, conformation, and/or the like) to the input antibody Ab.

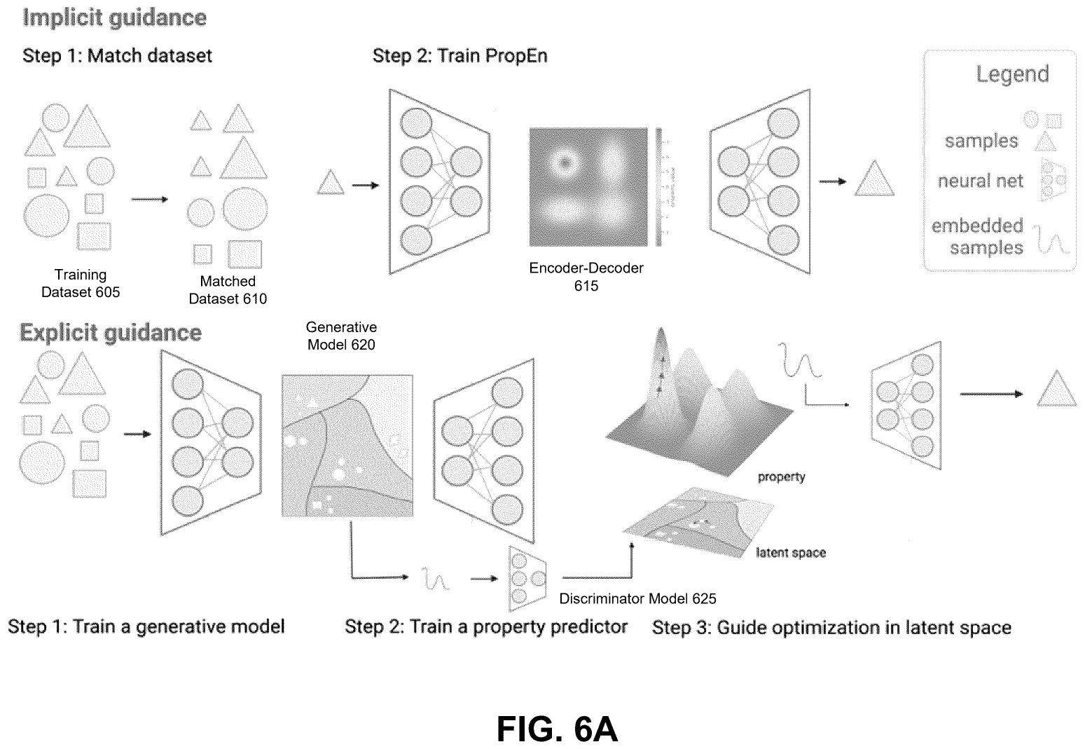

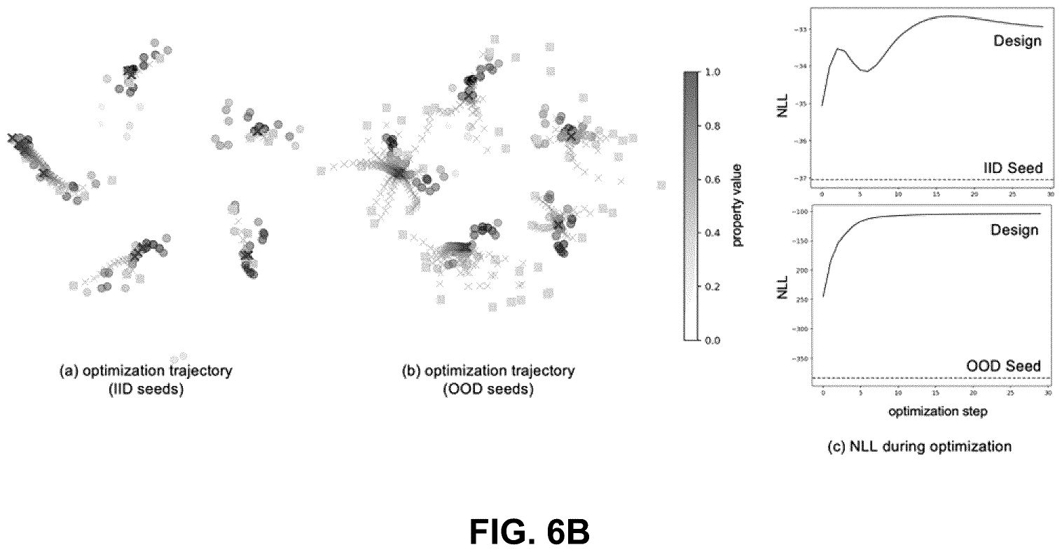

FIG. 6A depicts a schematic diagram illustrating a comparison of an example of an optimization task with implicit guidance and another example of an optimization task with explicit guidance, in accordance with some example embodiments. Referring to FIG. 6A, both tasks may include optimization the size of the objects, for example, by increasing the size of the objects. With the implicit guidance shown in the top panel, the samples in a training dataset 605 may be matched to generate a matched dataset 610 by at least pairing each sample in the training dataset 605 with another sample that is the closest match in terms of shape but with a superior property in terms of size. An encoder-decoder 615 (such as the one implementing the molecule design computation model 115 in FIG. 1) may be trained to encode one sample in each sample pair in the matched dataset into an embedding that is then decoded to recover the other sample in the same sample pair. In doing so, the encoder-decoder 615 may be trained to approximate a latent space (e.g., a lower dimensional manifold) where the embeddings are ordered by property value (e.g. size). Contrastingly, with the example of the optimization task with explicit guidance shown in the bottom panel, a generative model 620 is trained to generate objects while a discriminative model 625 trained to predict the property value (e.g., size) of objects guides the optimization in latent space.

FIG. 6B depicts a schematic diagram illustrating a comparison of an example of an optimization task with independent and identically distributed (IID) seed molecules and another example of a optimization task with out-of-distribution (OOD) seed molecules, in accordance with some example embodiments. In Panel (a), the optimization trajectories depict the changes in the properties of independent and identically distributed (IID) seed molecules over multiple design iterations. In Panel (b), the optimization trajectories depict the changes the changes in the properties of out-of-distribution (OOD) seed molecules over multiple design iterations. The graphs in Panel (c) depict the sum of negative log likelihoods of the seed molecules and optimized molecule designs across successive optimization steps.

FIG. 6B depicts the results of a synthetic example, the two-dimensional pinwheel dataset, which was used to illustrate the optimizing of designs with the encoder-decoder ƒ_{θ} (or the autoencoder F_{θ}). The property that was being optimized in this example was chosen as the log-likelihood of the data as estimated by a kernel density estimation (KDE) with Gaussian kernel with σ=0.01. In FIG. 6B, the training points are shown in Panels (a) and (b), with the color intensity representing the value of the property. As such, a higher/darker value is superior in this example. After training the encoder-decoder ƒ_{θ} (or the autoencoder F_{θ}), some points (squares) were held out and used as seed molecules. The x-markers represent molecule designs generated by the trained encoder-decoder ƒ_{θ} (or the autoencoder F_{θ}), with the color intensity increasing at each step t. Panel (c) of FIG. 6B shows that the molecule designs output by the trained encoder-decoder ƒ_{θ} (or the autoencoder F_{θ}) moves towards the regions of the training data with the highest property value, consistently improving with each step t. With out-of-distribution (OOD) seed molecules, Panel (b) shows that the trained encoder-decoder ƒ_{θ} (or the autoencoder F_{θ}) also elected to optimize them with molecule designs from the closest regions in the training data.

Molecule Design with Implicitly Guided Property Optimization

In some example embodiments, a molecule design computation model, such as the molecule design computation model 115 shown in FIG. 1, may be trained to optimize (or improve) one or more properties (e.g., drug-like properties) of an input molecule with implicit guidance. In some cases, this implicit guidance may be derived from a matched data set (e.g., the matched dataset 125 in FIG. 1) containing molecule pairs (e.g., the molecule pairs 170 in FIG. 1), each of which including two counterfactual molecules exhibiting sufficient similarities in molecular composition and/or conformation but different values for at least one property of interest. In some cases, the molecule design computation model may be trained to encode the input molecule and decode the resulting embedding to generate one or more output molecules exhibiting a different value (or superior value) for at least one of the properties present in the input molecule. That is, the molecule design computation model may be trained, based on observed samples x∈ [symbol] ^{m} from a distribution p, to sample new datapoints x* within a desired range of values for y*=g(x*), y∈ [symbol] for one or more properties of interest y. For example, in some cases, the molecule design computation model may be trained to operate on an input datapoint (x⁰, y⁰), which includes an input molecule x⁰ with the initial property values y⁰, and generate one or more new datapoints (x*, y*), each of which including an output molecule x* with a different property value y* (e.g., y*>y⁰). Doing so may be tantamount to training the molecule design computation model to approximate the data distribution q (x*|x, y) of the matched dataset subject to constraints. However, training the molecule design computation model to approximate the data distribution q(x*|x, y) based on a dataset

$$𝒟 = {\{ ( x , y ) \}}_{i = 1}^{n}$$

of n samples (or molecules), each of which including the features x and the labels (or property values) y, may be difficult for a number of reasons. At the outset, the dataset [symbol] may contain an inadequate amount of datapoints at least because measurements to obtain the labels (or property values) y are expensive and time consuming. Furthermore, the molecule x and the label (or property value) y may exhibit a highly complex functional dependency y=g(x), with the function g(x) being rigid and difficult to approximate in the absence of large quantities of data. Conventional solutions, such as Bayesian optimization, missing value imputation, and conditional guided sampling, are rarely successful due to the inability to adequately approximate the data distribution q(x*|x, y) from the available dataset [symbol] .

Training the molecule design computation model based on the molecule pairs in the matched dataset may mitigate the challenges associated with approximating the data distribution q (x*|x, y) from the available dataset [symbol] . As noted, when trained based on the molecule pairs in the matched dataset, the molecule design computation model may learn the causation (or dependence) between molecular features, such as certain compositional features and/or conformational features, and various molecular properties of interest (e.g., drug-like properties such as affinity, specificity, biological activity, developability, and/or the like). This is because each one of the molecule pairs may include two molecules that are counterfactuals. In the first molecular pair, for example, one molecule may be the most similar observed counterpart to the other molecule but exhibits a different value for at least one property.

e molecule design computation model may be trained to attribute the difference in the values of the at least one property to the differences, for example, in molecular composition, molecular conformation, and/or the like, that is present between the two molecules in the molecule pair.

To further illustrate, consider again the dataset

$$𝒟 = {\{ ( x , y ) \}}_{i = 1}^{n} ,$$

which can be transformed into a matched dataset ∀(x⁰, y⁰)∈ [symbol] in accordance with Equation (1) below.

$$( {x}^{0} , {y}^{0} ) \to {\{ ( {x}_{c}^{0} , {y}_{c}^{0} ) \}}_{c = 1}^{k} , s . t . d ( {x}^{0} , {x}_{c}^{0} ) < ϵ , {y}_{c}^{0} - {y}^{0} > δ \qquad ( 1 )$$

wherein ϵ, δ∈ [symbol] , and d denotes a proximity metric corresponding to the dataset [symbol] and the nature of the representations of the molecule x. As noted, in some cases, each molecule x, including its various compositional features and/or conformational features, may be represented in a number of different ways including as, for example, real data vectors, three-dimensional structure coordinates, image pixel representations, tokenized sequence molecule representations, and/or the like. According to Equation (1), two molecules x⁰ and

$${x}_{c}^{0}$$

may be matched as a pair if the proximity metric d between the two satisfies (e.g., is less than) a first threshold ϵ and the difference between their respective labels (or property values) y⁰ and

$${y}_{c}^{0}$$

satisfies (e.g., exceeds) a second threshold δ.

Another formulation of the matched dataset for every (x, y) within [symbol] is given by Equation (2) below.

$$ℳ = \{ ( x , {x}^{′} ) \vert \begin{matrix}x , {x}^{′} ∈ 𝒟 \\ {‖ {x}^{′} - x ‖}^{2} \leq {Δ}_{x} , g ( {x}^{′} ) - g ( x ) ∈ ( 0 , {Δ}_{y} )\end{matrix} \} , \qquad ( 2 )$$

wherein Δₓ and Δ_{y} are predefined positive thresholds. The matched dataset provides an extended collection of molecule pairs [symbol] whose size N=0 (n²)>>n may significantly exceed that of the training dataset, depending on the choice of matching thresholds Δₓ and Δ_{y}.

Upon generating the matched dataset, an encoder-decoder network ƒ_{θ} may be trained over [symbol] by minimizing the matched reconstruction objective:

$$ℓ ( {f}_{θ} ; ℳ ) = \frac{1}{\vert ℳ \vert} {\sum }_{( x , {x}^{′} ) ∈ ℳ} ℓ ( {f}_{θ} ( x ) , {x}^{′} ) ,$$

wherein [symbol] denotes an appropriate loss for the data in question, such as mean-squared error (MSE), cross-entropy loss, and/or the like. In some cases, reducing (or minimizing) the matched reconstruction objective yields a model that approximates the direction of the gradient of g(·), even if no property predictor has been explicitly trained. Thus, if ƒ* denotes the optimal solution of the matched reconstruction objective with a sufficiently small Δₓ, for any point x in the matched dataset for which p is uniform within a ball of radius Δₓ, ƒ*(x)→c∇g(x) for some positive constant c.

In some example embodiments, the molecule design computation model may be implemented as an autoencoder F_{θ} that includes an encoder coupled with a decoder. Accordingly, in some cases, the training of the molecule design computation model may include training the autoencoder F_{θ} based on the matched dataset ∀(x⁰, y⁰)∈ [symbol] while reducing (or minimizing) the loss function shown as Equation (3) below.

$$arg {{min}_{θ} ( {F}_{θ} ( {x}^{0} ) - {x}^{0} )}^{2} + {( {F}_{θ} ( {x}_{c}^{0} ) - {x}^{0} )}^{2} \qquad ( 3 )$$

The loss function shown as Equation (3) includes two terms. The first term corresponds to the reconstruction loss of the autoencoder F_{θ}, the reduction (or minimization) of which lends structure to the data manifold approximated by the autoencoder F_{θ} by at least constraining the distance between the input molecule x ingested by the autoencoder F_{θ} and the output molecule x′ generated by the autoencoder Fe therefrom. It should be appreciated that the first term may be optional. The second term lends order to the data manifold approximated by the autoencoder F_{θ} by imposing, implicitly through the construction of the matched dataset ∀(x⁰, y⁰)∈ [symbol] , a monotonicity constraint. Equation (4) below shows the second term independently.

$$arg {{min}_{θ} ( {F}_{θ} ( {x}_{c}^{0} ) - {x}^{0} )}^{2} \qquad ( 4 )$$

Referring now to Equation (4), the second term

$${( {F}_{θ} ( {x}_{c}^{0} ) - {x}^{0} )}^{2}$$

imposes a monotonicity constraint such that the autoencoder Fe preserves order. To further illustrate, recall that a function ƒ(·) is monotonically increasing (or non-decreasing) if for all x and y where x≤y, then ƒ(x)≤ƒ(y). When that is the case, the function ƒ(·) is said to preserve order. During the matching process to generate the matched dataset ∀(x⁰, y⁰)∈ [symbol] , each pairing of two molecules x⁰ and

$${x}_{c}^{0}$$

may be made such that the corresponding labels (or property values)

$${y}_{c}^{0} \geq {y}^{0} .$$

Given that y=g(x), the matched dataset ∀(x⁰, y⁰)∈ [symbol] may be generated such that

$$g ( {y}_{c}^{0} ) \geq g ( {y}^{0} ) .$$

In other words, the second term

$${( {F}_{θ} ( {x}_{c}^{0} ) - {x}^{0} )}^{2}$$

imposes a monotonicity constraint on the autoencoder F_{θ} such that

$${F}_{θ} ( {x}_{c}^{0} ) \geq {F}_{θ} ( {x}_{0} ) .$$

In large sample size, the autoencoder F_{θ} may approximate the function g (e.g., F_{θ}→g), or ϕ, some monotonic function. Monotonicity is preserved if, as shown below, the gradient of the function is positive.

$$\frac{∇ {F}_{θ} ( x )}{\partial x} > 0 or \frac{{F}_{θ} ( {x}^{′} ) - {F}_{θ} ( x )}{{x}^{′} - x} > 0$$

wherein x and x′ are two datapoints (or molecules) within the vicinity of one another in the data manifold. In order for the gradient of the function to be positive, the numerator and the denominator of the fractions above should have the same sign (e.g., both positive or both negative). While the denominator is positive due to the construction of the matched dataset ∀(x⁰, y⁰)∈ [symbol] in which x′>x, a positive nominator expresses the requirement for the autoencoder F_{θ} to reconstruct datapoints (or molecules) having superior (or higher) property values (e.g., F_{θ}(x′)>F_{θ}(x)). Assuming that the autoencoder F_{θ} is a high capacity model, it may be expected that F_{θ}(x′)˜x′ and F_{θ}(x)˜x. Given the construction of the matched dataset ∀(x⁰, y⁰)∈ [symbol] , the datapoint (or molecule) x′ may dominate the datapoint (or molecule) x in at least one dimension i where the datapoint (or molecule) x′ has a superior (or higher) value than the datapoint (or molecule) x.

In some cases, training on a matched dataset may permit auto-regressive sampling. Starting with a seed molecule x₀, for t=1, 2, . . . , successive molecule designs xₜ=ƒ_{θ}(xₜ₋₁) may be generated until convergence, ƒ_{θ}(xₜ)=xₜ, such that g(xₜ)>g(xₜ₋₁). For example, in some cases, the encoder-decoder ƒ_{θ} (or the autoencoder F_{θ}) may be applied to an initial seed molecule x₀ to generate a first molecule design x₁. In some cases, the encoder-decoder ƒ_{θ} (or the autoencoder F_{θ}) may be applied iteratively to its output until ƒ_{θ}(xₜ)=xₜ, which may be analogous to arriving at a stationary point at which ∇_{g} (xₜ)=0 and the direction of property enhancement is exhausted given the training data. In some cases, exploiting the implicit guidance from the matched dataset may result in a trajectory of multiple optimized molecule designs with superior properties than the initial seed molecule x₀.

In some cases, the molecule designs generated by the encoder-decoder ƒ_{θ} (or the autoencoder F_{θ}) may be almost as likely in the training set according to the data distribution p. This may serve as a guarantee that the generated molecule designs lie within the data distribution p. Thus, given a model ƒ* trained to reduce (or minimize) the matched reconstruction objective, the probability of ƒ*(x) may be at least

$$p ( {f}^{*} ( x ) ) \geq {𝔼}_{{x}^{′} ∼ {\hat{μ}}_{x}} [ p ( {x}^{′} ) ] - \frac{{‖ {H}_{p} ( f ( x ) ) ‖}_{2} {σ}^{2} ( {M}_{x} )}{2} ,$$

wherein \hat{μ}_{z} denotes the empirical measure on the dataset, Hₚ(x) is the Hessian of p at x, and

$${σ}^{2} ( {ℳ}_{x} ) = {𝔼}_{{x}^{′} ∼ {\hat{μ}}_{x}} [ {‖ {x}^{′} - {𝔼}_{{x}^{″} ∼ {\hat{μ}}_{x}} [ {x}^{″} ] ‖}_{2}^{2} ]$$

is the variance induced by the matching process.

In some example embodiments, the performance of the encoder-decoder ƒ_{θ} (or the autoencoder F_{θ}) in improving molecular properties may be evaluated for the six different molecular properties listed in Table 1 below.

TABLE 1

| | description | objective | dim | type |
|---|---|---|---|---|
| hydrophobicity | A measure of the relative tendency of an | minimize | 1 | structure |
| | analyte ‘to prefer’ a nonaqueous over an | | | |
| | aqueous environment. May be responsible | | | |
| | for changes in pharmacokinetics, efficacy, | | | |
| | dose interval, and application route. | | | |
| positive/negative | Patch integral, whole fragment antigen- | minimize | 1 | structure |
| charge | binding region (Fab) is taken into account. | | | |
| ab angle delta | The torsion angle between heavy and light | minimize | 1 | structure |
| | chain measured around carbon (C) atom. | | | |
| | Keep as close as possible to Ab angle of seed | | | |
| | molecule (e.g., 4D5). | | | |
| ab angle length | Ab length delta-length of carbon (C) atom. | minimize | 1 | structure |
| | Keep as close as possible to Ab angle length | | | |
| | of the seed molecule (e.g., 4D5). | | | |
| liability motifs | Liability motifs are collected. The goal is to | minimize | 3 | sequence |
| | minimize their count. Some liability motifs | | | |
| | may be prioritized based on importance, such | | | |
| | as with 1 being the most important (e.g., | | | |
| | tryptophan (W) to CDRs) metric across the | | | |
| | complimentarity determining region (CDR) | | | |
| | vicinity. | | | |
| immunogenicity | Immunogenicity may be calculated using | minimize | 3 | sequence |
| | NetMHCII, number of epic core proteins, | | | |
| | number of mighty core proteins, and the | | | |
| | number of strong core proteins. | | | |
| CDF | The multivariate rank can be computed | maximize | 6 | both |
| | across multiple properties of interest. The | | | |
| | Pareto front corresponds to a rank of 1.0 in | | | |
| | the maximization setup. | | | |
| hypervolume | Hypervolume (HV) is the polytope bounded | maximize | 6 | both |
| | from below by a reference point and from | | | |
| | above by the Pareto front. | | | |

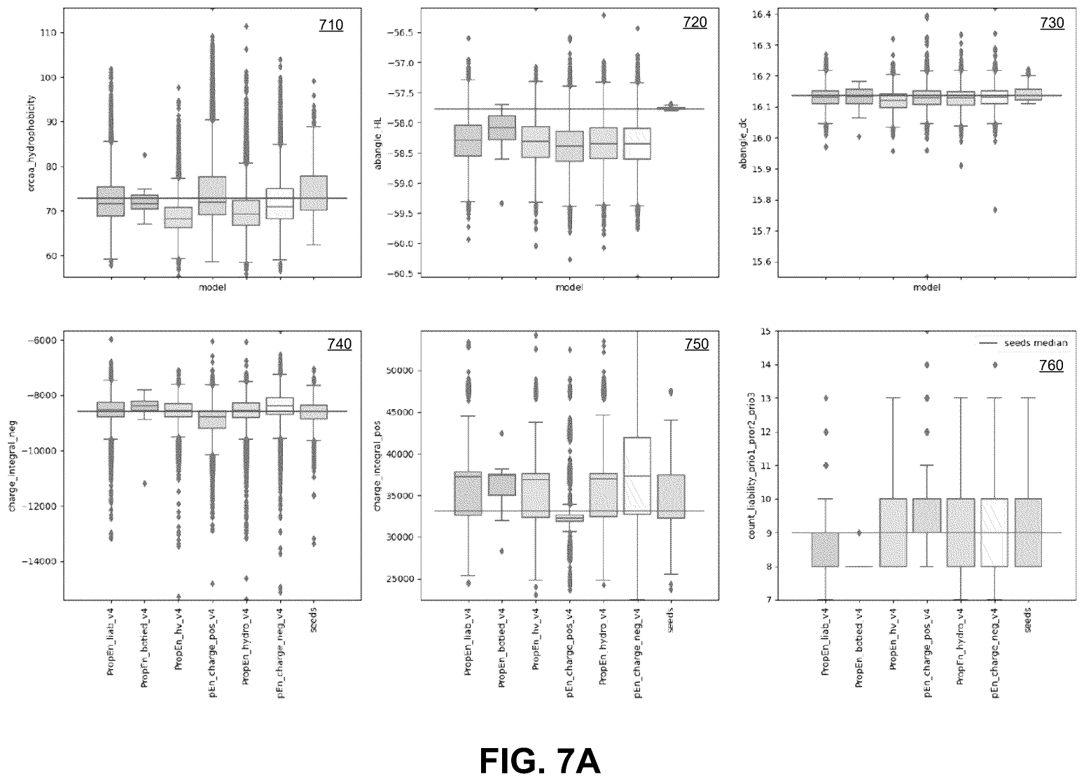

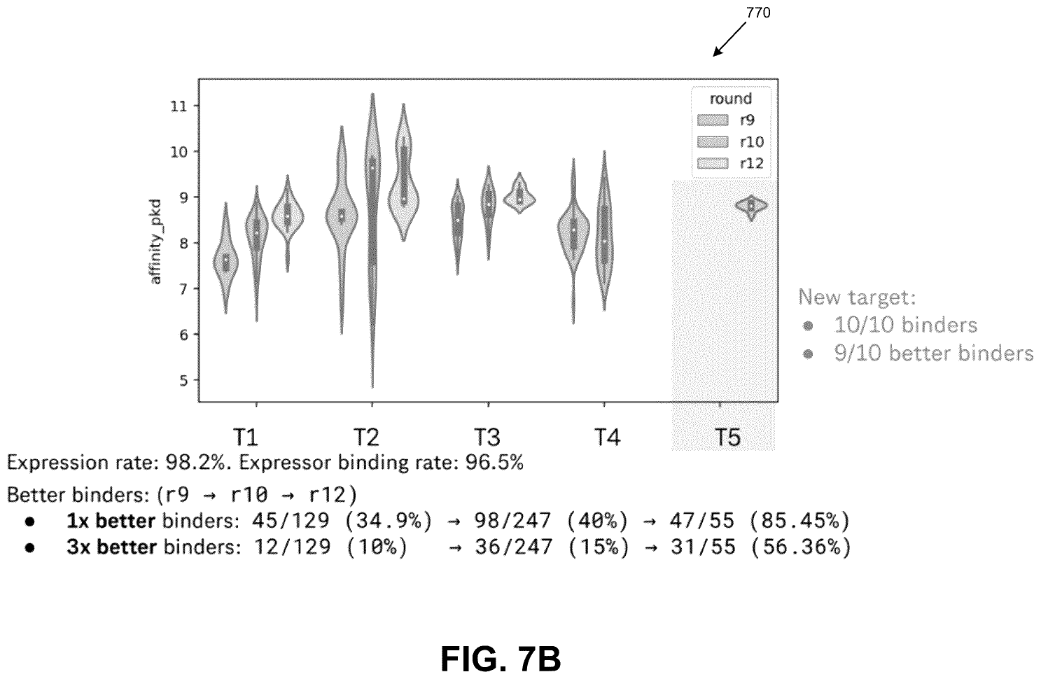

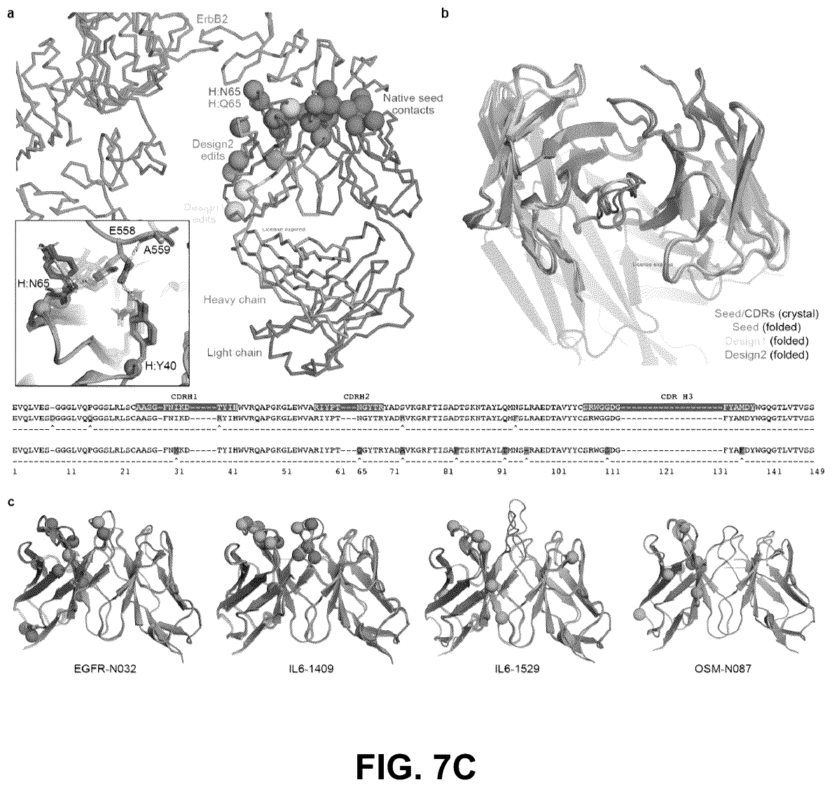

FIG. 7A depicts graphs illustrating a comparison of the performance of the encoder-decoder ƒ_{θ} (or the autoencoder F_{θ}) for improving the hydrophobicity (graph 710), ab angle delta (graph 720), angle angel length (graph 730), negative charge integral (graph 740), positive charge integral (graph 750), and liability motif count (graph 760) of molecules (e.g., antibodies). The graphs in FIG. 7A compares the properties of output molecules generated six different models as well as the properties of the seed molecules used to generate the output molecules. In particular, the graphs in FIG. 7A shows the properties of the output molecules generated by different versions of the encoder-decoder ƒ_{θ} (or the autoencoder F_{θ}). Some versions of the encoder-decoder ƒ_{θ} (or the autoencoder F_{θ}) were trained to improve specific properties, such as liability motifs, positive charge integral, and negative charge integral. Two versions of the encoder-decoder ƒ_{θ} (or the autoencoder F_{θ}) were trained to improve multiple properties by optimizing multivariate ranks such as cumulative density function (CDF) indicator and hypervolume (HV). As shown in FIG. 7, the best performing versions of the encoder-decoder ƒ_{θ} (or the autoencoder F_{θ}), as indicated by the properties of the output molecules, are those trained for specific properties. For example, the version of the encoder-decoder ƒ_{θ} (or the autoencoder F_{θ}) trained to improve negative charge integral outperformed the others when it comes to generating output molecules having a superior negative charge integral than the seed molecules. Likewise, the version of the encoder-decoder ƒ_{θ} (or the autoencoder F_{θ}) trained to improve liability motifs outperformed the others when it comes to reducing the number of liability motifs present in the seed molecules.

FIG. 7B depicts a violin plot 770 illustrating the performance of the encoder-decoder ƒ_{θ} (or the autoencoder F_{θ}) in improving binding affinity towards five different targets T1, T2, T3, T4, and T5 over successive design iterations, in accordance with some example embodiments Referring to FIG. 7B, the violin plot 770 includes wet lab binding affinity measurements of the output molecules (e.g., antibodies) generated by the encoder-decoder ƒ_{θ} (or the autoencoder F_{θ}). For the first four targets T1, T2, T3, and T4, the encoder-decoder ƒ_{θ} (or the autoencoder F_{θ}) generated output molecules with consistently superior binding affinity over successive design iterations. For the fifth target T5, the encoder-decoder ƒ_{θ} (or the autoencoder F_{θ}) is able to generate output molecules with 3× superior binding affinity than the seed molecule over just a single design iteration, without the benefit of more, improved training data from previous design rounds. An example of an output molecule generated by the encoder-decoder ƒ_{θ} (or the autoencoder F_{θ}), which has 30× stronger binding affinity than the lead molecule, is shown in FIG. 7C.

Molecule Design with Implicitly Guided Conditional Generation

In some example embodiments, a molecule design computation model, such as the molecule design computation model 115 shown in FIG. 1, may be trained to generate output molecules with improved (or superior) values for one or more properties of interest with implicit guidance. In some cases, this implicit guidance may also be derived from a matched data set (e.g., the matched dataset 125 in FIG. 1) containing molecule pairs (e.g., the molecule pairs 170 in FIG. 1), each of which including two counterfactual molecules exhibiting sufficient similarities in molecular composition and/or conformation but different values for at least one property of interest. As shown in the top panel of FIG. 6A, conventional data-driven approaches to property optimization rely on explicit guidance from a discriminative model trained on available data to approximate an unknown objective landscape. In this paradigm, the discriminative model may either rank or select molecule designs generated by a generative model to guide the generative process towards molecule designs exhibiting one or more desired properties. Nevertheless, when data for training the discriminative model is scarce, which is often the case in scientific applications such as drug design, training a reliable discriminative model may be infeasible. Furthermore, drug design tasks often seek designs from a different data distribution than the available training data, meaning that the discriminative model is required to make even less reliable out-of-distribution inferences.

In some cases, various example embodiments of the molecule design computation model described in the present disclosure overcome the limitations of conventional data-driven approaches to property optimization by leveraging implicit guidance in a sampling-based generative paradigm. For example, in some cases, the molecule design computation model may be trained to generate an output molecule having a superior value for at least one property of interest by at least denoising a noise molecule while being conditioned on an input molecule having an inferior value for the at least one property of interest. In some cases, the molecule design computation model may be trained based on a matched dataset to approximate the data distribution (or matched distribution) from which to sample the output molecule. Whereas the molecule design computation model trained to perform implicitly guided property optimization generates a single output molecule with improved property values for each input molecule, the molecule design computation model trained to perform implicitly guided generation learns an entire data distribution (or matched distribution) of diverse output molecules with improved property values.

In some example embodiments, the implicitly guided generation approach may leverage the pairwise relationships that exist within the matched dataset. These pairwise relationships include matching samples x exhibiting lower-valued properties with samples x′ exhibiting higher-valued properties. Doing so may implicitly define a target distribution with improved property values (or an improved target distribution). In some cases, this “matching” strategy may form a conditional density estimation problem in which a generative model is compelled, for example, via maximum likelihood training or approximating score functions, to reproduce the distribution of samples with improved property values, thereby obviating the discriminative model used in conventional data-driven approaches.

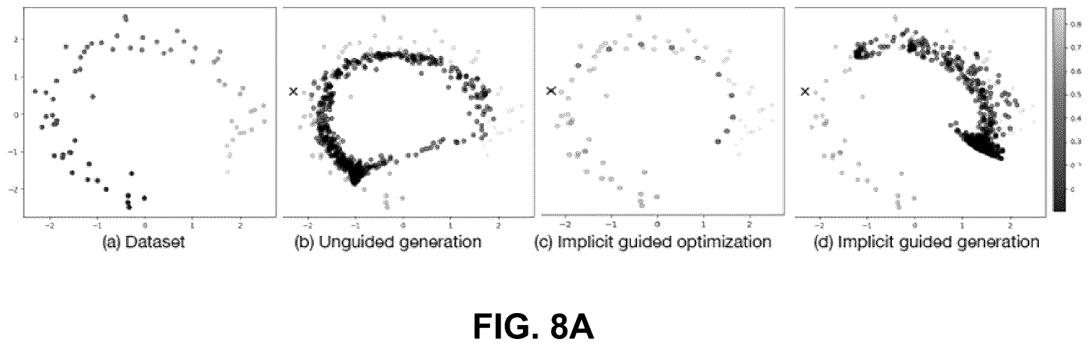

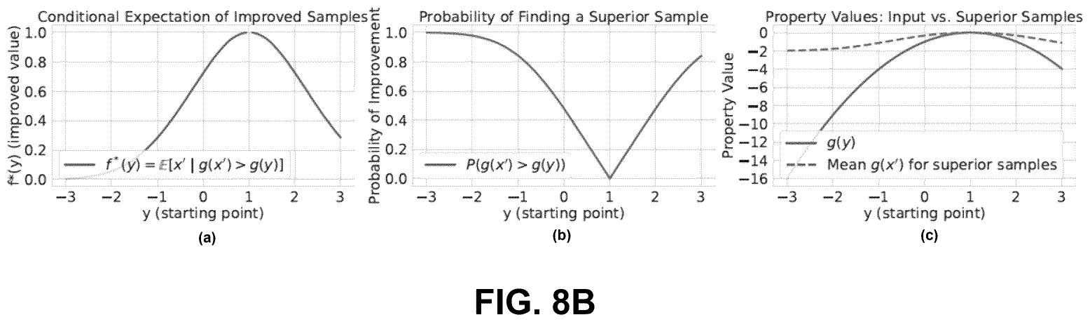

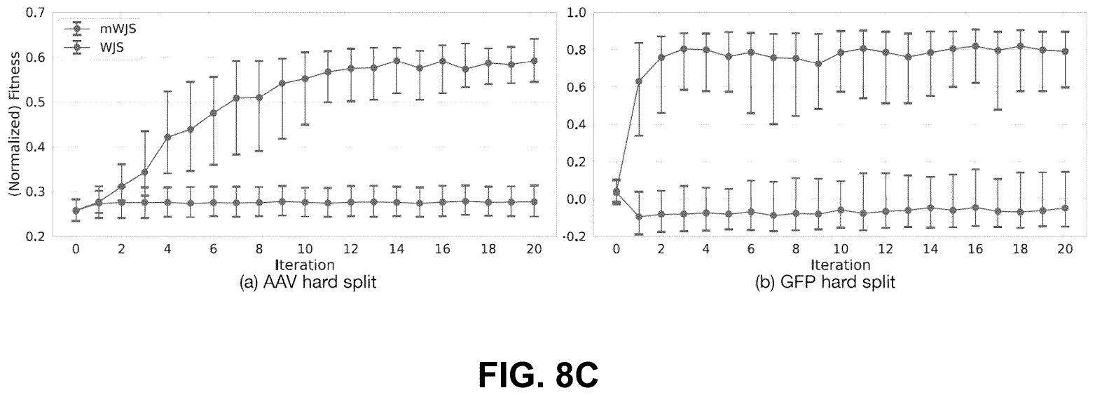

To further illustrate the differences between the aforementioned approaches, FIG. 8A depicts an illustrative two-dimensional dataset (in Panel (a)) and a comparison of the results of unguided conditional generation (in Panel (b)), implicitly guided property optimization (in Panel (c)), and implicitly guided conditional generation with increasing values of the property (in Panel (d)). The value of the property of the illustrative two-dimensional dataset (in Panel (a)) increases clockwise. The datapoints (marked as black dots) sampled by an unconditional (or unguided) generative model trained on this dataset are shown in Panel (b). The unconditional (or unguided) generative model is unaware of the properties present in the sampled datapoints. Thus, compared to the seed design (marked as a cross in Panel (b)), the sampled datapoints may exhibit lower as well as higher property values. Contrasting, with a generative model trained for implicitly guided optimization, each datapoint sampled by the generative model constitutes a separate optimization step and generates a design having higher property values than the seed design (marked as a cross in Panel (c)). Finally, in Panel (d), a generative model trained for implicitly guided conditional generation samples diverse datapoints (marked as black dots) with increasing property values compared to the initial seed design (marked as a cross).

In some example embodiments, the molecule design computation model, such as the molecule design computation model 115 shown in FIG. 1, may be trained to generate output molecules given one or more input molecules (or seed molecules) with the objective that the output molecules are similar to the input molecules (or seed molecules) but exhibiting superior values in one or more properties of interest. For example, in some cases, the molecule design computation model may be trained to generate to improve the binding affinity (or another property) of an input molecule by at least generating one or more output molecules with superior binding affinity than the input molecule but within a limited quantity of edit distances. In other words, given a d-dimensional input molecule x∈ [symbol] ⊂ [symbol] ^{d} with ground truth property value g(x)∈ [symbol] , the molecule design computation model may be applied to generate output molecules x′∈ [symbol] ^{d} such that (i) g(x′)−g(x)<∇_{g} and (ii) dist(x′, x)<∇ₓ, for predefined positive thresholds ∇_{g} and ∇ₓ. In some cases, this objective may be cast as a conditional generation problem that includes training the molecule design computation model to sample from the data distribution (or matched distribution) of output molecules with improved property values given the input molecule x:

$${p}^{+} ( {x}^{′} \vert x ) ∝ p ( {x}^{′} ) 𝕀 [ g ( {x}^{′} ) > g ( x ) ∧ dist ( {x}^{′} , x ) < {Δ}_{x} ] ,$$

wherein [symbol] is the indicator function.

Let p(x) denote a probability distribution defined over x∈ [symbol] ^{d}, Δₓ∈ [symbol] ⁺, and let g: [symbol] ^{d}→ [symbol] be any function inducing a binary ordering via the following:

$$𝕀 ( {x}^{′} , x ) = \{ \begin{matrix}1 , & g ( {x}^{′} ) > g ( x ) ∧ dist ( {x}^{′} , x ) < {Δ}_{x} , \\ 0 , & otherwise ,\end{matrix}$$

and define the improved conditional distribution as:

$${p}^{+} ( {x}^{′} \vert x ) = \frac{p ( {x}^{′} ) 𝕀 ( x , {x}^{′} )}{Z ( x )} , Z ( x ) = \int p ( {x}^{′} ) 𝕀 ( x , {x}^{′} ) {dx}^{′}$$

Consider the functional:

$$ℒ ( q ) = - {𝔼}_{x ∼ p ( x )} [ {𝔼}_{{x}^{′} ∼ {p}^{+} ( {x}^{′} \vert x )} [ log q ( {x}^{′} \vert x ) ] ] ,$$

wherein q(·|x) may be any family of conditional densities. The unique minimizer of [symbol] (q) under the normalization constraint ∫q(x′|x)dx′=1 is the following:

$$q * ( {x}^{′} \vert x ) = {p}^{+} ( {x}^{′} \vert x ) .$$

To further illustrate, consider a one-dimensional example in which the data distribution p(x) is the standard normal and the property of interest is defined as g(x)=−(x−1)². For a given suboptimal input molecule y exhibiting an inferior value g(y) for the property of interest, the improved distribution may be defined as p⁺(x′|y)∝p(x′) [symbol] (g(x′)>g(y)). The following may be computed: the conditional expectation ƒ*(y)≈ [symbol] [x′|g(x′)>g(y)], the probability p(g(x′)>g(y)), and the comparison of the quality g(y) to the average quality of the output molecules exhibiting improved values for the property of interest. FIG. 8B depicts graphs illustrating the results of the one-dimensional example. In Panel (a), which shows the conditional expectation of output molecules x′ with improved property values (or ƒ*(y)≈ [symbol] [x′|g(x′)>g(y)]), the observation is that when the suboptimal input molecule y is far from the optimal region (x≈1), the expectation of superior output molecules x′ is significantly higher. Contrastingly, as the suboptimal input molecule y is near the optimal region, the improvement diminishes. In Panel 9(b), which shows the probability of generating the output molecule x′ to exhibit superior property values than the input molecule y (or p(g(x′)>g(y))), the observation is that the likelihood of generating a superior output molecule x′ decreases as the input molecule y approaches the optimal region (x≈1). Finally, in Panel (c), which shows a comparison of the property values present in the input molecule y and the superior output molecules x′ (or [symbol] [x′|g(x′)>g(y)]), the observation is that the improved output molecules x′ not only exhibit higher property values but also consistently exhibit enhanced quality g(x′) relative to that of the input molecule g(y).

In some example embodiments, the molecule design computation model may be a conditional generator q_{θ}(x′|x), parameterized by θ, that approximates the improved distribution p⁺(x′|x). In some cases, to achieve this training objective, the conditional generator q_{θ}(x′|x) may be trained while reducing (or minimizing) the following loss function shown as Equation (5) below.

$$ℒ ( θ ) = - {𝔼}_{x ∼ p ( x )} [ {𝔼}_{{x}^{′} ∼ {p}^{+} ( {x}^{′} \vert x )} log {q}_{θ} ( {x}^{′} \vert x ) ] \qquad ( 5 )$$

In some cases, the reduction (or minimization) of the loss function [symbol] (θ) may align the generative process with the desired improvement in property value through conditional density estimation, thereby bypassing the need for an explicit discriminative model.

In some cases, given an initial dataset

$$𝒟 = {\{ ( {x}_{i} , g ( {x}_{i} ) ) \}}_{i = 1}^{n} ,$$

xᵢ∈ [symbol] ^{d}, g(xᵢ)∈ [symbol] , and the constraint Δₓ∈ [symbol] ⁺, a matched dataset

$$ℳ = {\{ {x}_{i}^{′} , {x}_{i} \}}_{i = 0}^{N}$$

(with N>>n usually and ideally) is constructed in accordance with Equation (6) below.

$$ℳ = \{ {x}_{i}^{′} , {x}_{i} ∈ {ℝ}^{d} \times {ℝ}^{d} : dist ( {x}^{′} - x ) \leq {Δ}_{x} , g ( {x}^{′} ) > g ( x ) \} \qquad ( 6 )$$

wherein x and x′ denote sample molecules from the dataset [symbol] . In some cases, by construction, the matched dataset [symbol] may contain samples drawn from the improved distribution p⁺(x′|x).

As described in more details below, the conditional generator q_{θ}(x′|x) implementing the molecule design computation model may be adapted with a variety of different machine learning architectures. A matched variational autoencoder (mVAE) is one example architecture in which the conditional generator q_{θ}(x′|x) is instantiated as a latent-variable model parameterized by θ(ϕ, ψ) and defined by Equation (7).

$${q}_{θ} ( {x}^{′} \vert x ) = \int {p}_{ψ} ( {x}^{′} \vert x , z ) {q}_{ϕ} ( z \vert x , {x}^{′} ) dz . \qquad ( 7 )$$

In some cases, training the matched variational autoencoder (mVAE) may include increasing (or maximizing) the evidence lower bound (ELBO) defined by Equation (8).

$${𝒥}_{mVAE} ( θ , ℳ ) = {\sum }_{( x , {x}^{′} ) ∈ ℳ} {𝔼}_{{q}_{ϕ} ( z \vert x , {x}^{′} )} log {p}_{ψ} ( {x}^{′} \vert x , z ) - KL ( {q}_{ϕ} ( z \vert x , {x}^{′} ) \vert \vert 𝒩 ( 0 , {I}_{d} ) ) , \qquad ( 8 )$$

wherein [symbol] (0, I_{d}) is the standard d-dimensional normal distribution. Optimizing this training objective is a variational relaxation of the maximum likelihood problem. In some cases, with sufficient capacity and data, the decoder distribution p_{ψ}(x′|x, z) may converge to the improved distribution p⁺(x′|x). Accordingly, the matched variational autoencoder (mVAE) may realize the optimal density while providing a tractable latent representation and sampling mechanism. In some cases, output molecules may be sampled following a conditional variational autoencoder approach. For example, given an input molecule x, z may be sampled from the prior before being forwarded through the decoder to derive the approximated output molecules from

$${p}_{ψ} ( {x}^{′} \vert x , z ) .$$

Another example of the machine learning architecture implementing the conditional generator q_{θ}(x′|x). With guided flow matching, samples may be generated from the improved conditional distribution given an input molecule by (i) sampling first from an (easy) source distribution p₀ before (ii) transforming that sample into another sample from the desired target distribution by solving an ordinary differential equation (ODE). In some cases, a time-dependent velocity field

$${v}_{t}^{θ} ( {x}_{t}^{′} , x )$$

may be parameterized with ∈[0,1] and

$${x}_{t}^{′}$$

being an intermediate sample from the probability path pₜ(·|x) (e.g., a linear path) that defines the velocity field. In some cases, the velocity field may be learned by adapting the conditional flow matching loss to the setting defined in Equation (9), which may match the parameterized velocity to the optimal velocity that transports the source distribution p₀ to the probability path probability path pₜ(·|x).

$${𝒥}_{mFM} ( θ , ℳ ) = {\sum }_{( x , {x}^{′} ) ∈ ℳ} {𝔼}_{t ∼ U ( 0 , 1 ) , {x}_{0}^{′} ∼ {p}_{0}} {‖ {v}_{t}^{θ} ( {x}_{t}^{′} , x ) - ( {x}^{′} - {x}_{0}^{′} ) ‖}^{2} \qquad ( 9 )$$

It should be appreciated that since the data being modeled is discrete, the discrete flow matching loss can also be used. In some cases, minimizing the loss in Equation (9) (or its discrete counterpart) may yield the improved conditional distribution. In some cases, once the velocity field is learned, output molecules with improved property values may be sampled therefrom by solving the ordinary differential equation

$$( ODE ) {x}_{t}^{′} = {v}_{t}^{θ} ( {x}_{t}^{′} , x ) ,$$

wherein

$${x}_{0}^{′} ∼ {p}_{0} and {x}_{1}^{′}$$

is an (approximate) sample from the improved distribution p⁺(x′|x).

As yet another possible alternative, the conditional generator q_{θ}(x′|x) may be implemented with a score-based generative model, such as a matched walk-jumped sampler (mWJS), that approximates samples from the improved distribution p⁺(x′|x) by (i) sampling noisy samples from the smooth distribution p(y|x)=p⁺(x′|x)* [symbol] (0, σ²I_{d}) containing a (usually high) noise level σ and (ii) estimating the corresponding clean samples \hat{x}= [symbol] (x|y). In some cases, a conditional walk-jump sampling framework may be adopted in which a conditional denoiser is trained before being used to approximate noisy samples from the p⁺(x′|x). In some cases, the conditional denoiser D_{θ}: [symbol] may be a neural network parameterized by θ that takes, as input, pairs (y, x) and outputs an estimated clean version of x′ (or the clean version of the noisy sample). In some cases, the denoiser may be trained by minimizing the loss function in Equation (10).

$${𝒥}_{mWJS} ( θ , ℳ ) = {\sum }_{( x , {x}^{′} ) ∈ ℳ} {𝔼}_{ε ∼ 𝒩 ( 0 , {σ}^{2} {I}_{d} )} {‖ {D}_{θ} ( {x}^{′} + σε , x ) - {x}^{′} ‖}^{2} \qquad ( 10 )$$

In some cases, once the denoiser is trained, the score function p(y|x) may be approximated using the conditional version of the Tweedie-Miyasawa formula

$$\begin{matrix}∇ log p ( y \vert x ) \approx \\ {s}_{θ} ( y \vert x ) = \frac{1}{{σ}^{2}} ( {D}_{θ} ( y , x ) - y ) .\end{matrix}$$

In some cases, the trained conditional denoiser D_{θ} and the corresponding score function s_{θ} may be leveraged to generate output molecules x′ conditioned on input molecules x in accordance with the following;

(i) (init.) select an input molecule x and initialize an initial noisy sample y₀ with the addition of noise.

(ii) (walk) sample noisy designs yₖ˜p(y|x) with a gradient based sampling technique such as Langevin Markov Chain Monte Carlo (MCMC):

$${y}_{k + 1} = {y}_{k} + δ {s}_{θ} ( y \vert x ) + \sqrt{2 δ} ε , ε ∼ 𝒩 ( 0 , {I}_{d} ) .$$

(iii) (jump) generate clean samples at arbitrary step K:

$${K}_{} = {D}_{θ} ( {y}_{K} , x ) .$$

In some example embodiments, the conditional generator q_{θ}(x′|x) may be applied in an iterative manner to sample output molecules with improved properties constrained by Δₓ. For example, in some cases, the sampling procedure may be applied multiple times, guiding output molecules towards higher property values, until one or more criteria is met (e.g., quantity of design iterations, threshold value for the property, and/or the like). It should be appreciated that the iterative optimization approach still does not rely on any auxiliary discriminative models. Moreover, with each design iteration, the conditional generator q_{θ}(x′|x) may generate a population of output molecules.

In some cases, the iterative design process may be defined as

$${x}_{k + 1}^{′} ∼ {p}^{+} ( \cdot \vert {x}_{k}^{′} ) ,$$

k=1, 2, . . . , Nᵢₜₑᵣ. Starting from an input molecule

$${x}_{0}^{′} ← x$$

(or a set of input molecules), the conditional generator q_{θ}(x′|x) may be applied to generate a population of output molecules with improved property values after a single design iteration, after which the output molecules are used as input molecules for the following design iteration. The output molecules from design iteration k+1 should exhibit a superior property value than the output molecule from the previous design iteration k, while still being “close” in the input space.

Algorithm 1 below describes the pseudo-code implementing the iterative design process.

Algorithm 1: Iterative Sampling

```
Data: Model qθ, seeds $𝒮 = {\{ {x}_{i} \}}_{i = 1}^{n} ,$
n iterations of Nᵢₜₑᵣ, n samples per iteration M, distance
threshold δ n designs N.
Result: Designs ${\{ {x}_{i}^{′} \}}_{i = 1}^{N} .$
  for i ← 0 to Nᵢₜₑᵣ do
  // 1. Sample M designs given [symbol]
  $𝒟 = {\{ {x}_{i}^{′} \}}_{i = 1}^{M} ∼ {q}_{θ} ( \cdot \vert 𝒮 ) ;$
  // 2. rejection step [symbol]
  ← unique( [symbol] ); [symbol]
  ← {x|dist(x, \tilde{x}) < δ, x ∈ [symbol] , \tilde{x} ∈ [symbol] };
  // 3. Update seeds [symbol]
  ← [symbol]
end for [symbol]
← choose_random( [symbol] , N) // Select N random samples
from pool of designs
return [symbol]
```

In some cases, the performance of the conditional generator q_{θ}(x′|x) may be improved by leveraging pseudo-matches. For example, in some cases, starting from a small population of true (input, output) molecule pairs, the output molecules that are generated by the conditional generator q_{θ}(x′|x) may be paired back with the original input molecule to form pseudo-matched molecule pairs. Accordingly, each pseudo-matched (or synthetic) molecule pair may include an original input molecule and an output molecule generated by the conditional generator q_{θ}(x′|x) operating on the input molecule. In some cases, these pseudo-matched (or synthetic) molecule pairs may be added to augment the matched dataset used to train the conditional generator q_{θ}(x′|x). In some cases, multiple, iterative training cycles with pseudo-matching may be performed in which output molecules generated by the conditional generator q_{θ}(x′|x) are pseudo-matched to generate pseudo-matched (or synthetic) molecule pairs that are then used to retrain the conditional generator q_{θ}(x′|x). In some cases, pseudo-matching, for example, over multiple iterative training cycles, may improve the generative performance of the conditional generator q_{θ}(x′|x) by at least enabling the conditional generator q_{θ}(x′|x) to refine its conditional distribution and explore regions of the output space beyond the original data manifold. For instance, in some cases, the conditional generator q_{θ}(x′|x) may learn from its “best” output molecules, increase its ability to produce high-value output molecules, and converge quicker on more extreme property values that could not have been achieved with the limited population of true (input, output) molecule pairs that was originally available for training the conditional generator q_{θ}(x′|x).

The performance of the conditional generator q_{θ}(x′|x) was evaluated in two settings. First, the output molecules generated by the conditional generator q_{θ}(x′|x) were compared with output molecules from baseline models using a protein fitness optimization in silico benchmark, which focuses on subdomains of adeno-associated virus (AAV) and green fluorescent protein (GFP) proteins. Second, iterative training was leveraged to push the boundaries of the values of the properties of the output molecules beyond their scope in the matched dataset used to train the conditional generator q_{θ}(x′|x).

As noted, the performance of the conditional generator q_{θ}(x′|x) was evaluated by comparing its output molecules against the output molecules from baseline models using the protein fitness optimization in silico benchmark focusing on the subdomains of adeno-associated virus (AAV) and green fluorescent protein (GFP) proteins. The former measures the ability of the adeno-associated virus (AAV) to package a deoxyribonucleic acid (DNA) payload (e.g., for applications such as gene delivery) while the latter's fitness is its fluorescence (e.g., useful for biomarkers and/or the like). The adeno-associated virus (AAV) protein and the green fluorescent protein (GFP) datasets respectively contain 44,156 and 56,806 pairs of amino acid sequences and experimentally measured properties.

The benchmark that was used contains functional segments of lengths L=28 and L=237 amino acid residues for adeno-associated virus (AAV) protein and green fluorescent protein (GFP) respectively. The protein sequences were one-hot encoded over a dictionary of 20 amino acid residues. The optimal fitness set for each dataset was defined as the top 99^{th} percentile of experimental data with the highest measured property. The datasets were split two ways with 2,139/3,448 samples for adeno-associated virus (AAV) and 2,828/2,426 for green fluorescent protein (GFP). The “medium” split constitutes a subset of the data containing the 20^{th} to 40^{th} percentiles that are 6 edit distances (or mutations) or more from any sample in the optimal fitness set. The “hard” split contains the lowest 30^{th} percentiles that are 7 or more edit distances (or mutations) away from the optimal fitness set. The matched dataset used to train the conditional generator q_{θ}(x′|x) was generated using Levenshtein distance and applied a Δₓ of 5 and 10 for the adeno-associated virus (AAV) dataset and the green fluorescent protein (GFP) dataset, respectively.

The baseline models in this comparison include GFlowNets (GFN-AL), model-based adaptive sampling (CbAS), greedy search (AdaLead), Bayesian optimization (BO-qei), conservative model-based optimization (CoMs), and proximal exploration (PEX). The evaluation started with the optimization process in which 128 input molecules (or seed molecules), X={xᵢ} are used to generate 128 output molecules,

$${X}^{′} = \{ {x}_{i}^{′} \} ,$$

superior fitness than the input molecules (or seed molecules). The output molecules were generated iteratively over Nᵢₜₑᵣ=20 design iterations. At each design iteration, a pool of M=2560 designs were sampled and repeats and those that have a Levenshtein distance larger than τ=10 from any input molecule (or seed molecule) were rejected from advancing to the next design iteration. On the final design iteration, 128 samples were randomly selected as output molecules from the last remaining pool of designs. The fitness of the output molecules were approximated using a fitness predictor (or pseudo-oracle), ĝ. The same fitness predictor ĝ was implemented as a one-dimensional convnet trained on all wet-lab data. The fitness was min-max normalized on all tasks.

The following metrics were computed for the set of output molecules: Fitness=median ({ĝ(x′ᵢ)|x′∈X′}), the median of the approximated fitness predicted by the fitness predictor ĝ, Diversity=median({dist(x, \tilde{x})|x, \tilde{x}∈X′, x≠\tilde{x}}), the median of the Levenshtein distance between every pair of output molecules (amino acid sequences) generated by the models, and

$$Novelty = median ( {\{ η ( {x}_{i}^{′} , X ) \}}_{i = 1}^{128} ) ,$$

wherein η(x, X)=min({dist(x, \tilde{x})|x, \tilde{x}∈X′, x≠\tilde{x}}) is the minimum distance to sample x to any starting input molecule in X. The objective was to generate output molecules that scores higher in terms of the fitness metric, while diversity and novelty metrics are shown to assess the tradeoff between exploration (of unknown designs) and exploitation (of known designs). For example, a random output molecule (with a random amino acid sequence) would exhibit high diversity and novelty but its fitness metric would be unreliable.

Table 1 depicts the adeno-associated virus (AAV) benchmark results, with the mean/standard deviation across 5 runs (in parenthesis).

TABLE 1

| | Medium Difficulty | | | | | Hard Difficulty | | | | |
|---|---|---|---|---|---|---|---|---|---|---|
| Method | Fitness | Diversity | | Novelty | | Fitness | Diversity | | Novelty | |
| GFN-AL | 0.20 (0.1) | 9.6 | (1.2) | 19.4 | (1.1) | 0.10 (0.1) | 11.6 | (1.4) | 19.6 | (1.1) |
| CbAS | 0.43 (0.0) | 12.7 | (0.7) | 7.2 | (0.4) | 0.36 (0.0) | 14.4 | (0.7) | 8.6 | (0.5) |
| AdaLead | 0.46 (0.0) | 8.5 | (0.8) | 2.8 | (0.4) | 0.40 (0.0) | 8.5 | (0.1) | 3.4 | (0.5) |
| BOqei | 0.38 (0.0) | 15.2 | (0.8) | 0.0 | (0.0) | 0.32 (0.0) | 17.9 | (0.3) | 0.0 | (0.0) |
| CoMS | 0.37 (0.1) | 10.1 | (5.9) | 8.2 | (3.5) | 0.26 (0.0) | 10.7 | (3.5) | 10.0 | (2.8) |
| PEX | 0.40 (0.0) | 2.8 | (0.0) | 1.4 | (0.2) | 0.30 (0.0) | 2.8 | (0.0) | 1.3 | (0.3) |
| GWG | 0.43 (0.1) | 6.6 | (6.3) | 7.7 | (0.8) | 0.33 (0.0) | 12.0 | (0.4) | 12.2 | (0.4) |
| GGS | 0.51 (0.0) | 4.0 | (0.2) | 5.4 | (0.5) | 0.60 (0.0) | 4.5 | (0.5) | 7.0 | (0.0) |
| mVAE | 0.48 (0.02) | 9.5 | (0.3) | 6.0 | (0.0) | 0.38 (0.04) | 12.04 | (1.2) | 7.3 | (0.8) |
| mFM | 0.52 (0.01) | 6.2 | (0.2) | 5.6 | (0.6) | 0.35 (0.02) | 6.6 | (0.3) | 5.2 | (0.5) |
| mWJS | 0.53 (0.01) | 5.2 | (0.2) | 5.6 | (0.6) | 0.54 (0.04) | 4.6 | (0.7) | 6.6 | (0.5) |

Table 2 depicts the green fluorescent protein (GFP) benchmark results, with the mean/standard deviation across 5 runs (in parenthesis).

TABLE 2

| | Medium Difficulty | | | | | Hard Difficulty | | | | |
|---|---|---|---|---|---|---|---|---|---|---|
| Method | Fitness | Diversity | | Novelty | | Fitness | Diversity | | Novelty | |
| GFN-AL | 0.09 (0.1) | 25.1 | (0.5) | 213 | (2.2) | 0.10 (0.2) | 23.6 | (1.0) | 214 | (4.2) |
| CbAS | 0.14 (0.0) | 9.7 | (1.1) | 7.2 | (0.4) | 0.18 (0.0) | 9.6 | (1.3) | 7.8 | (0.4) |
| AdaLead | 0.56 (0.0) | 3.5 | (0.1) | 2.0 | (0.0) | 0.18 (0.0) | 5.6 | (0.5) | 3.4 | (0.5) |
| BOqei | 0.20 (0.0) | 19.3 | (0.0) | 0.0 | (0.0) | 0.00 (0.5) | 94.6 | (71) | 54.1 | (81) |
| CoMS | 0.00 (0.1) | 133 | (25) | 192 | (12) | 0.00 (0.1) | 144 | (7.5) | 201 | (3.0) |
| PEX | 0.47 (0.0) | 3.0 | (0.0) | 1.4 | (0.2) | 0.00 (0.0) | 3.0 | (0.0) | 1.3 | (0.3) |
| GWG | 0.10 (0.0) | 33.0 | (0.8) | 12.8 | (0.4) | 0.00 (0.0) | 4.2 | (7.0) | 7.6 | (1.1) |
| GGS | 0.76 (0.0) | 3.7 | (0.2) | 5.0 | (0.0) | 0.74 (0.0) | 3.6 | (0.1) | 8.0 | (0.0) |
| mVAE | 0.XX | 0.XX | (0.2) | 0.XX | (0.5) | 0.78 (0.04) | 1.27 | (0.2) | 7.5 | (0.6) |
| mFM | 0.50 (0.03) | 5.3 | (0.2) | 7.0 | (0.0) | 0.55 (0.04) | 5.4 | (0.1) | 7.7 | (0.5) |
| mWJS | 0.76 (0.03) | 3.2 | (0.1) | 6.0 | (0.0) | 0.78 (0.02) | 2.9 | (0.2) | 7.0 | (0.0) |

The results in Table 1 and 2 compare the performance of the conditional generator q_{θ}(x′|x), implemented with different machine learning architectures (matched variational autoencoder (mVAE), matched flow matching (mFM), and matched walk-jump sampler (mWJS) against the baseline models across the medium and hard difficulty splits of the adeno-associated virus (AVV) dataset and the green fluorescent protein (GFP) dataset, respectively. As shown, different variations of the conditional generator q_{θ}(x′|x) achieved comparable or superior results as most baseline models, while being conceptually simpler and without any reliance on a separator discriminator model. In particular, the best performing variation of the conditional generator q_{θ}(x′|x), the matched walk-jump sampler (mWJS), achieved competitive results with the best performing baseline model GSS and outperformed the other baseline models by a large margin, particularly for the “hard” split.

FIG. 8C depicts a comparison of the performance of the conditional generator q_{θ}(x′|x) implemented as a matched walk-jump sampler (mWJS), the best performing model against the baselines, and the performance of its unmatched (or unconditional) counterpart (WJS). Panel (a) shows a comparison of the performance of the two models operating on the adeno-associated virus (AAV) dataset and Panel (b) shows a comparison of the performance of the two models operating on the green-fluorescent protein (GFP) dataset. It should be appreciated that the two models have the same architecture and hyperparameters but the matched walk-jump sampler (mWJS) is conditioned on a matched dataset while the unmatched walk-jump sampler (WJS) is an unconditional model. As shown in Panel (a) and (b), the medium fitness of the output molecules generated by both models evolves as more optimization steps are performed but the median fitness of the output molecules from the matched walk-jump sampler (mWJS) increases with more design iterations while the median fitness of the output molecules from the unmatched walk-jump sampler (WJS) remains relatively fixed.

Molecule Design with Implicitly Guided Property Optimization and Joint Representations

In some example embodiments, a molecule design computation model, such as the molecule design computation model 115 shown in FIG. 1, may be trained to operate on a joint representation of an input molecule that combines multiple representations of the input molecule. For example, as described in more details below, a structural encoder may be trained to generate, based at least on a linear representation of the input molecule, the joint representation of the input molecule by at least incorporating the corresponding two- or three-dimensional structural context information. In instances where the input molecule is a protein molecule, for example, the joint representation of the input molecule may combine the amino acid sequence of the input molecule with a three-dimensional representation of the input molecule that specifies at least a portion of the three-dimensional structure (or conformation) adopted by the folding of the amino acid sequence. Alternatively, in instances where the input molecule is a chemical compound, the joint representation of the input molecule may combine a linear (or one-dimensional) representation of the input molecule (e.g., a simplified molecular-input line-entry system (SMILES) string, an international chemical identifier (InChI), a SELF-referencing embedded string (SELFIES)) with a two-dimensional representation (e.g., a molecular graph) and/or a three-dimensional representation (e.g., point cloud, atomic density field) of the input molecule.

In some cases, the joint representation of the input molecule may incorporate structural context information that is unavailable in the linear (or one-dimensional) representation of the input molecule. As such, in some cases, operating on the joint representation of the input molecule may improve the performance of the molecule design computation model or extend the capabilities of the molecule design computation model to zero- or one-shot optimization and/or generation. In some cases, even though the linear (or one-dimensional) representation of the input molecule is out-of-distribution (OOD) relative to the sample molecules in the matched dataset used to train the molecule design computation model, the two- or three-dimensional representation of the input molecule may nevertheless exhibit similar biophysical patterns as the sample molecules in the matched dataset. For example, in instances where the input molecule is a protein molecule, the amino acid sequence of the input molecule may be out-of-distribution (OOD) of the matched dataset but the three-dimensional structure (or conformation) adopted by the amino acid sequence may nevertheless exhibit similar biophysical patterns as the sample molecules in the matched dataset. Accordingly, despite not having encountered a sample molecule with a similar amino acid sequence during training, the molecule design computation model may nevertheless be capable of generating, based on the joint representation of the input molecule, one or more output molecules with superior values for at least one property of interest.

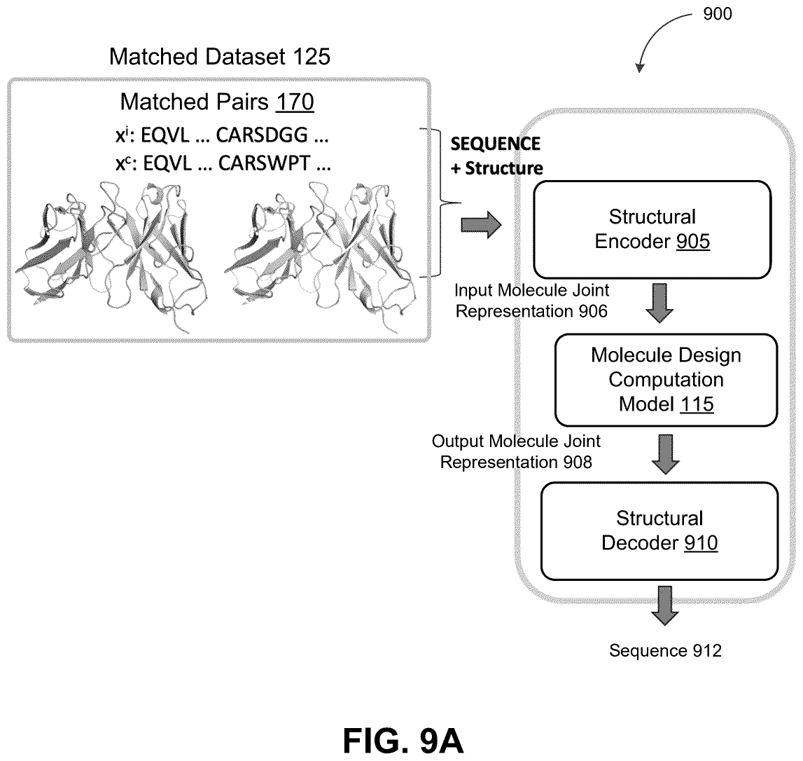

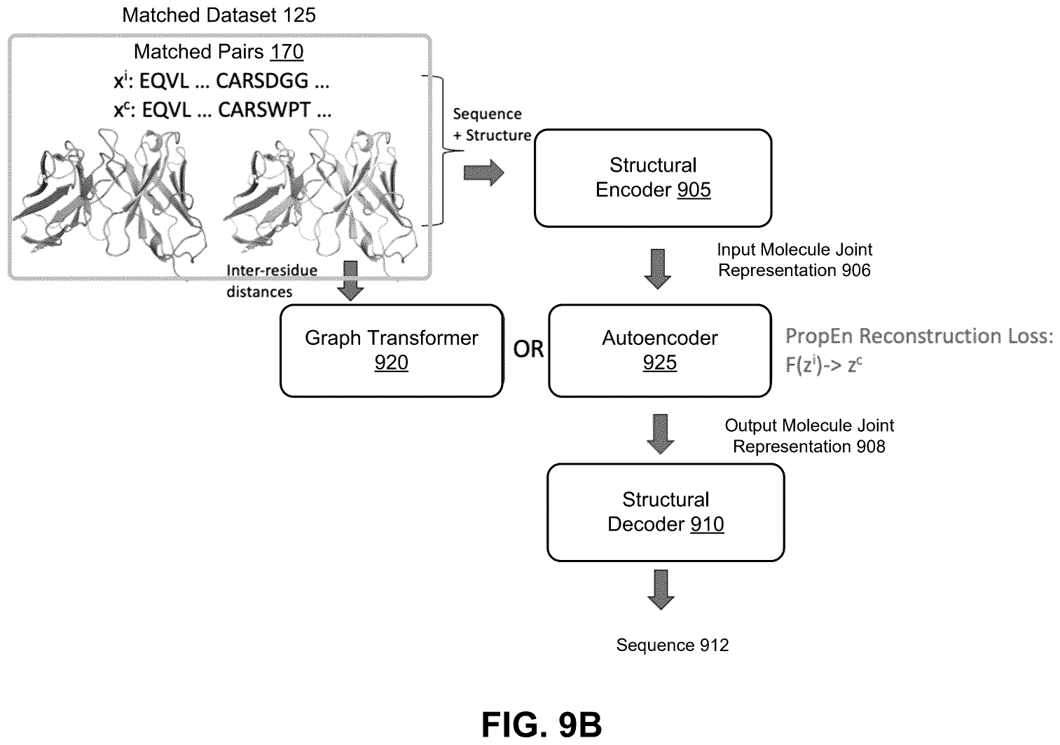

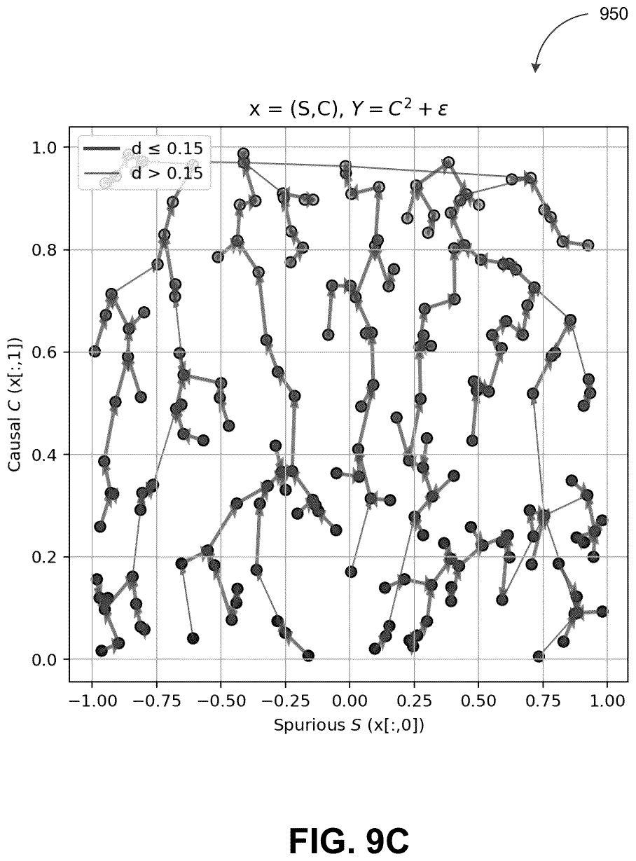

To further illustrate, FIG. 9A depicts a schematic diagram illustrating an example of a process 900 for machine learning enhancement of one or more molecular properties, in accordance with some example embodiments. Referring to FIG. 9A, in some cases, the matched dataset 125 may include multiple representations of the counterfactual molecules forming each molecule pair 170. For example, in the context of protein design, each of the matched pairs 170 in the matched dataset 125 may include, for the constituent counterfactual molecules, the amino acid sequence and the three-dimensional structure (or conformation) adopted by the amino acid sequence. In some cases, the matched dataset 125 may be used to train the molecule design computation model 115 as well as a structural encoder 905 and a structural decoder 910. For instance, in some cases, the structural encoder 905 may be trained to generate, based on the amino acid sequence of an input molecule, a joint representation 906 of the input molecule while the structural decoder 910 may be trained to recover, from the joint representation 908 of an output molecule generated by the molecule design computation model 115, the amino acid sequence 912 of the output molecule.

In some cases, where the input molecule and the output molecule are protein molecules, the joint representation 906 of the input molecule as well as the joint representation 908 of the output molecule may be per-residue embeddings that includes, for each constituent amino acid residue, structural context information. In some cases, the structural context information may specify, for each constituent amino acid residue, one or more adjacent amino acid residues. For example, in some cases, the structural context information may include an adjacency matrix that identifies, for each constituent amino acid residue, one or more other amino acid residues that located are within a threshold distance of each other in three-dimensional space. In some cases, the molecule design computation model 115 may generate the joint representation 908 of the output molecule by at least encoding the joint representation 906 of the input molecule before the resulting embedding is decoded to generate the joint representation 908 of the output molecule. Alternatively, the molecule design computation model 115 may generate the joint representation 908 of the output molecule by at least denoising a noise molecule while being conditioned on the joint representation 906 of the input molecule.

FIG. 9B depicts two examples of machine learning architectures that can be used for implementing the molecule design computation model 115. As shown in FIG. 9B, in some cases, the molecule design computation model 115 may be implemented as a graph transformer 920 (e.g., a graph neural network (GNN)) trained using the inter-residue distances present in the matched dataset 125. Alternatively, the molecule design computation model 115 may be implemented as an autoencoder 925 (a convolutional neural network (CNN) based autoencoder or another encoder-decoder architecture) trained by at least reducing (or minimizing) the reconstruction loss present in the joint representation 908 of the output molecule. In some cases, for a molecule pair (e.g., from the matched dataset 125) containing the joint representation Zᵢ of a first sample molecule and the joint representation Z_{c} of a second sample molecule. the aforementioned reconstruction loss may be defined as Equation (11) below.

$$ℒ = f ( {\hat{Z}}_{c} , {Z}_{c} ) \qquad ( 11 )$$

wherein \hat{Z}_{c} denotes the joint representation that is generated by the molecule design computation model 115 operating on the joint representation Zᵢ of the first sample molecule. As shown in Equation (11), in some cases, the training objective for the molecule design computation model 115 may be to reduce (or minimize) the difference between the joint representation \hat{Z}_{c} and the original joint representation Z_{c} of the second sample molecule from the molecule pair.

As noted, in some example embodiments, a molecule design computation model, such as the molecule design computation model 115 shown in FIG. 1, may leverage the combination of features from one-dimensional (or linear) space and those from two- and/or three-dimensional spaces to achieve superior performance in one-shot scenarios in which the one-dimensional (or linear) representation of the input molecule is not in the distribution of the sample molecules in the matched dataset used to train the molecule design computation model. In the context of protein design, for example, the molecule design computation model may leverage the combination of features in sequence space and features in structural space to incrementally transform a lower affinity antibody to a higher affinity antibody, In some cases, this incremental transformation may be performed in a one-shot manner, meaning that the lower affinity antibody may be out-of-distribution (OOD) by being dissimilar to the sample antibodies used to train the molecule design computation model. In some cases, the transformation of the lower affinity antibody may be iterative in that the output molecule from one design iteration performed by the molecule design computation model on the lower affinity antibody may be cycled back and used as the input molecule during a subsequent design iteration.

To further illustrate, let [symbol] denote the space of protein sequences (e.g., antibody sequences) and let [symbol] ⊂ [symbol] denote the measurements for a property of interest, such as binding affinity towards an antigen. Access to E environments (or “seeds”) indexed by e=1, . . . . E is assumed, wherein each environment corresponds to a distinct lead molecule (e.g., lead antibody) and, in some cases, a particular antigen context. In environment e, a small subset of protein sequences with measured properties {

$$( {x}_{j}^{e} , {y}_{j}^{e} )$$

} is observed, with

$${y}_{j}^{e} ∈ 𝓎 .$$

The objective is, for a held-out new environment e* (e.g., the “one-shot” seed molecule), to generate a set of new designs

$${\{ {\hat{x}}_{i}^{{e}^{*}} \}}_{k = 1}^{K} ⊂ 𝒳$$

that reliably improve on the property present in the lead molecule

$${y}_{lead}^{{e}^{*}} .$$

This objective is achieved by:

1. Form matched pairs

$$( {x}_{j}^{e} , {x}_{j}^{′ e} )$$

in each training environment e by identifying, for each low-affinity sequence

$${x}_{i}^{e} ,$$

the nearest neighbor

$${x}_{i}^{′ e}$$

with a superior value for the property of interest

$$( or {y}_{i}^{′ e} > {y}_{i}^{e}$$

under a capped edit-distance threshold δₓ.

2. Train a shared encoder: ϕ: [symbol] → [symbol] ^{k} and decoder ƒ: [symbol] ^{k}→ [symbol] that maps the low-affinity sequence

$${x}_{i}^{e}$$

to its higher-affinity nearest neighbor

$${x}_{i}^{′ e} ( or {x}_{i}^{e} ↦ {x}_{i}^{′ e} )$$

across multiple environments e=1, . . . , E.

3. At test (or validation) time, apply the encoder-decoder (ϕ, ƒ) pair to the held-out environment e* to generate candidate molecule designs

$${\tilde{x}}_{i}^{{e}^{*}} = f ( ϕ ( {x}_{lead}^{{e}^{*}} ) )$$

with superior values for the property despite the seed environment e* being out-of-distribution (OOD).

Under mild smoothness and margin assumptions on the embedding and matching, the foregoing one-shot “matching interventions” procedure is able to provably recover the underlying causal features of the property (e.g., binding affinity) and generalize to out-of-distribution seed molecules (e.g., seed antibodies). Given the space of observed protein sequences (e.g., antibody sequences) [symbol] , endowed with a metric ∥·∥ (e.g., Euclidean on embeddings or edit distance). As earlier, E environments (e.g., seed molecules or antigen contexts) are indexed by e=1, . . . . E, with a small labeled subset of protein sequences with measured properties {

$$( {x}_{j}^{e} , {y}_{j}^{e} )$$

} being observed in each environment e. A structural causal model (SCM) is constructed as follows:

$$x = g ( C , S ) , y = h ( C ) + ε , ε ⊥ ( C , S ) ,$$

wherein C∈ [symbol] ^{d}^{_{c}} denote the causal features driving the value of a property (e.g., binding affinity) via a Lipschitz map h, and S∈ [symbol] ^{d}^{ₛ} denote the spurious features that vary a cross environments e∈ε. The functions g and h remain invariant across environments.

A two-dimensional toy example demonstrating the effects of matching interventions is shown in the graph 950 depicted in FIG. 9C. In FIG. 9C, each point x=(S, C) has a “spurious” coordinate S˜Uniform(−1, 1) and a “causal” coordinate C˜Uniform(−1, 1). However, the observed property in this example is nonlinearly related: Y=sin(2πC)+ε, ε˜ [symbol] (0, 0.1²). For each point x in FIG. 9C, an arrow is drawn to its nearest neighbor with a strictly higher property value Y. The arrow rendered in one color (light gray) if the edit distance ∥x′−x∥ between two points is below a threshold δₓ (valid match) or a different color (dark gray) to otherwise (pruned match). Even under this nonlinear, noisy relationship, FIG. 9C shows that the arrows indicating a valid match move almost entirely along the vertical, meaning that they hold the spurious variable S nearly constant while increasing the causal variable C. Thus, thresholded matching on x “simulates” an intervention denoted as do(C⬆) that affects the true casual factors C. This empirical separation of causal shifts from spurious variations underpins the theory that by reconstructing precisely those arrows indicating a valid match, for example, with a shared encoder-decoder (ϕ, ƒ) pair, can guarantee an injective mapping that ignores spurious noise while yielding provable gains in the value of the property Y for new environments, such as out-of-distribution (OOD) seed molecules and antigen contexts. Three assumptions are relevant for the foregoing theory:

Assumption 1 (Bi-Lipschitz Generative Map): The map g→ [symbol] ^{d}^{_{c}}^{+d}^{ₛ}→ [symbol] is bi-Lipshitz on its support, meaning there exits constants 0<α≤β<∞ such that

$$α ‖ ( C , S ) - ( {C}^{′} - {S}^{′} ) ‖ \leq ‖ g ( C , S ) - g ( {C}^{′} , {S}^{′} ) ‖ \leq β ‖ ( C , S ) - ( {C}^{′} , {S}^{′} ) ‖ ∀ C , \text{} {C}^{′} , S , {S}^{′} .$$

Assumption 2 (Lipschitz Affinity Function): The true affinity map h→ [symbol] → [symbol] is Lₕ-Lipschitz, or |h(C)−h(C′)|≤Lₕ∥C-C′|∀C, C′.

Assumption 3 (One-to-Many Matching): For each environment e and each low-affinity sequence

$${x}_{i}^{e} = g ( {C}_{i}^{e} , {S}_{i}^{e} )$$

with measured property value

$${y}_{i}^{e} ,$$

there exists a non-empty set of higher-affinity neighbors

$${ℳ}_{i}^{e} = \{ {x}_{i , 1}^{′ e} , … , {x}_{i , {K}_{i}}^{′ e} \}$$

with property value

$${y}_{i , j}^{′ e} > {y}_{i}^{e} , d ( {x}_{i}^{e} , {x}_{i , j}^{′ e} ) \leq {δ}_{{n}_{ε}} ,$$

wherein δₙ_{_{ε}}→0 as the library size n_{ε}→∞. Moreover, any two distinct targets across multiple matched datasets satisfy a margin

$$‖ {x}_{i , j}^{′ e} - {x}_{i , k}^{′ e} ‖ \geq m > 0 , ∀ ( i . j ) \neq ( {i}^{′} , k ) .$$

In some cases, the multi-target reconstruction objective is achieved by training an encoder ∥ [symbol] → [symbol] and a decoder ƒ: [symbol] [symbol] under the constraint k<dim( [symbol] ) and Lipschitz constants ∥ϕ∥_{Lip}≤L_{ϕ}, ∥ƒ∥_{Lip}≤L_{ƒ}. The per-environment reconstruction risk is defined as follows:

$${R}_{e} ( ϕ , f ) = {𝔼}_{i ∼ e} [ \frac{1}{{K}_{i}} {\sum }_{j = 1}^{{K}_{i}} {‖ f ( ϕ ( {x}_{i}^{e} ) ) - {x}_{i , j}^{′ e} ‖}^{2} + λ {‖ f ( ϕ ( {x}_{i}^{e} ) ) - {x}_{i}^{e} ‖}^{2} ] ,$$

wherein λ≥0 weights on optional self-reconstruction term.

The worst-case risk can be optimized across different environments as follows:

$$( \hat{ϕ} , \hat{f} ) = {argmin}_{\begin{matrix}{‖ ϕ ‖}_{Lip} \leq {L}_{ϕ} \\ {‖ f ‖}_{Lip} \leq {L}_{f}\end{matrix}} {max}_{e = 1 , … , E} {R}_{e} ( ϕ , f ) .$$

Given Assumptions 1-3 above, the trained encoder-decoder pair (\hat{ϕ}, \hat{ƒ}) is able to achieve a maximum reconstruction error of less than

$$\frac{{m}^{2}}{4}$$

as indicated below:

$${R}_{max} = {max}_{e} {R}_{e} ( \hat{ϕ} , \hat{f} ) < \frac{{m}^{2}}{4} ,$$

in which case the trained encoder \hat{ϕ} is injective on the set of training inputs

$$\{ {x}_{i}^{e} \} .$$

As an injective function provides a one-to-one mapping, it should be appreciated that the injective trained encoder \hat{ϕ} maps distinct protein sequences (e.g., antibody sequences) to distinct embeddings (or codes).

The trained encoder \hat{ϕ} is provably injective because the contrary scenario in which two distinct training inputs x and x′ are mapped to the same embedding (or z=\hat{ϕ}(x)=\hat{ϕ}(x′) contradicts the margin criterion under Assumption 3. That is, under Assumption 3, x has targets

$$\{ {x}_{j}^{′} \}$$

and x′ has targets

$$\{ {x}_{k}^{′} \} ,$$

all of which are pairwise separated by at least the margin m. The decoder reconstructs each target from code z, such that for any such pair

$$f ‖ f ( z ) - {x}_{j}^{′} ‖ < \sqrt{{R}_{max}} and ‖ f ( z ) - {x}_{k}^{′} ‖ < \sqrt{{R}_{max}} .$$

By the triangle inequality,

$$‖ {x}_{j}^{′} - {x}_{k}^{′} ‖ \leq ‖ {x}_{j}^{′} - f ( z ) ‖ + ‖ f ( z ) - {x}_{k}^{′} ‖ < 2 \sqrt{{R}_{max}} < m ,$$

which contracts the margin requirement

$$‖ {x}_{j}^{′} - {x}_{k}^{′} ‖ \geq m .$$

Accordingly, the trained encoder \hat{ϕ} is injective.

Furthermore, because the trained encoder-decoder pair (\hat{ϕ}, ƒ) minimizes worst-case reconstruction risk during training, for any out-of-distribution environment e*, the reconstruction error satisfies the following:

$${R}_{e} * ( \hat{ϕ} , \hat{f} ) \leq {R}_{max} .$$

By the same margin argument, no collapse can occur in the novel environment e*, meaning that injectivity and low reconstruction error also holds for out-of-distribution (OOD) scenarios. Under the additional assumption that each matched latent shift yields at least Δₕ gain up to approximation error ρₙ_{ₑ}, it can be shown that by training a single encoder-decoder pair (ϕ, ƒ) to reconstruct matched low property value→high property value molecule pairs across diverse environments, the encoder can be guaranteed to be one-to-one (or injective) on meaningful sequence variations. Furthermore, the learned mappings will generalize with bounded error to entirely novel environments, such as novel seed molecules (e.g., seed antibodies), ensuring that the trained encoder-decoder pair (\hat{ϕ}, \hat{ƒ}) captures true causal features associated with the property (e.g., binding affinity) rather than spurious noise. The gain in the value of the property (e.g., binding affinity), or latent-gain approximation, can also be bound under the additional assumption (or latent-gain approximation) that for each low-affinity latent C matched to C′,

$$h ( {C}^{′} ) - h ( C ) \geq {Δ}_{h} > 0 , ‖ {C}^{′} - C - ‖ \leq {ρ}_{{n}_{e}} , {ρ}_{{n}_{e}} \to 0 .$$

Under Assumptions 1-3 and the latent-gain approximation assumption, the trained encoder \hat{ϕ} is provably guaranteed to improve the value of the property. Given the following,

$${R}_{max} = {max}_{e ∈ ε} {}_{i ∼ e} [ {‖ f ( ϕ ( {x}_{i} ) - {x}_{i}^{′} ) ‖}^{2} ] \leq {ε}^{2} .$$

Then for any test input x=g(C, S) in a new environment e*, the proposed design \tilde{x}=ƒ(ϕ(x)) has true latent C(\tilde{x}) satisfying the following

$$h ( ( \tilde{x} ) ) - h ( C ) \geq {Δ}_{h} - \frac{{L}_{h}}{α} ε - {L}_{h} {ρ}_{{n}_{{e}^{*}}} ,$$

wherein Δₕ denotes the minimal ideal gain and ρₙ_{ₑ}_{*} quantifies the matching approximation error.

The foregoing establishes that one-to-many matching combined with multi-target reconstruction (i) enforces an injective encoding of training protein sequences, (ii) generalizes with bounded error to new environments, and (iii) yields provable affinity gains under mild smoothness and margin assumptions. By training the encode-decoder (ϕ, ƒ) pair to succeed at reconstructing matched molecule pairs drawn from a variety of spurious-shift environments, the model may be trained to ignore features of the protein sequence, or the spurious “noise” S that fluctuates arbitrarily across environments (e.g., seed molecules or antigen contexts), that do not consistently explain changes in property (e.g., increases in binding affinity). What remains in the shared, low-dimensional embedding ϕ(x) are those latent directions in the causal factors C that drive the changes in the property (e.g., binding affinity) across multiple, or in some cases, every environment (e.g., antigen). As a result, the decoder ƒ is trained to amplify the truly causal components of the causal factors C, thus ensuring that the modifications suggested by the model is able to reliably improve the property (e.g., binding affinity) in novel settings (e.g., novel antigens). This invariance-by-matching principle not only yields a provably injective representation of the protein sequence's (e.g., antibody's) casual factors, but directly drives sample-efficient affinity maturation process that is robust against out-of-distribution input sequences (e.g., seed antibodies) in practice.

As noted, assuming a small-margin separation between distinct matched-targets, and with a low-dimensional, Lipschitz-constrained bottleneck on the encoder-decoder (ϕ, ƒ) pair, the trained encoder-decoder (\hat{ϕ}, \hat{ƒ}) pair with maximum reconstruction risk

$${R}_{max} ( \hat{ϕ} , \hat{f} ) < \frac{{m}^{2}}{4}$$

would satisfy the following:

1. Injectivity: \hat{ϕ} is one-to-one on the training support.

2. Exact Reconstruction: each matched pair is recovered up to

$$error < \frac{{m}^{2}}{4} .$$

3. OOD Transfer: for any new environment e* whose spurious support is within distance η of the training supports, the reconstruction risk is quantified as

$${R}_{e} * ( \hat{ϕ} , \hat{f} ) \leq {R}_{max} ( \hat{ϕ} , \hat{f} ) + O ( {( \frac{β}{α} )}^{2} {η}^{2} ) + o ( 1 ) .$$

The guarantees with respect to injectivity, exact reconstruction, and out-of-distribution (OOD) transfer rely on two metrics in latent space: (i) the matching error δₙ_{ₑ} (or a mismatch in which two non-counterfactual molecules are paired as such), and (ii) the separation margin m between distinct matched targets. In pure sequence space where the encoder-decoder (ϕ, ƒ) operates on the protein sequence alone (without any structural context information), the landscape of g(C, S) data distribution may be jagged. For example, small changes in either (or both) causal features C and spurious features S may engender large changes in the protein sequence (e.g., edit-distance shifts). Consequently, different high-affinity protein sequences may end up arbitrarily close in the data distribution of g(C, S), collapsing m [symbol] 0 and spoiling injectivity. Contrastingly, by embedding each protein sequence (e.g., antibody) via a structural encoder, such as the structural encoder shown in FIGS. 9A-B, g is effectively replaced by x [symbol] ψ(g(C, S)), wherein ψ is Lipschitz-smooth over both the causal features C and the spurious features S. Empirically, the incorporation of structural context information may reduce matching error δₙ_{ₑ} because neighbors in the structural space may remain close under ψ even when if the corresponding protein sequences exhibit a large edit distance. Furthermore, the incorporation of structural context information may amplify the margin m between distinct higher-affinity three-dimensional protein structures (or conformations), since ψ separates them by folding-aware geometric features. When combined, a small

$$\frac{{δ}_{{n}_{e}}}{α}$$

and a large margin m may preserve injectivity, exact reconstruction, and out-of-distribution (OOD) transfer to yield an injective encoder ϕ and strong out-of-distribution (OOD) bounds that generally do not exist when operating on protein sequence alone.

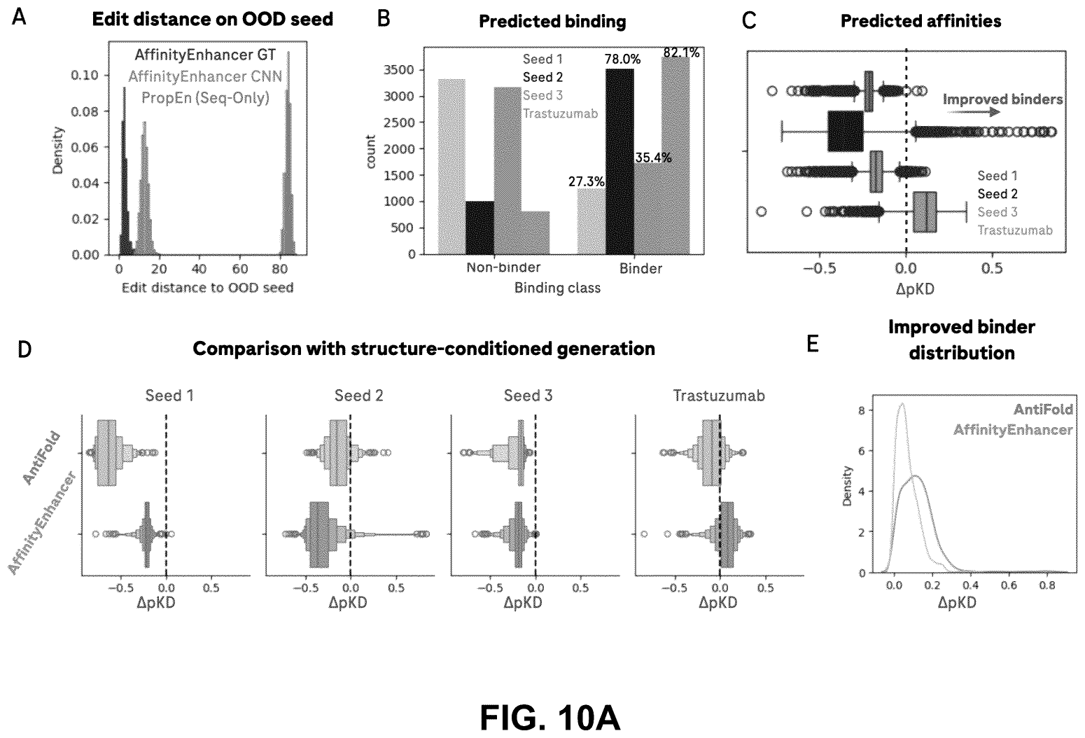

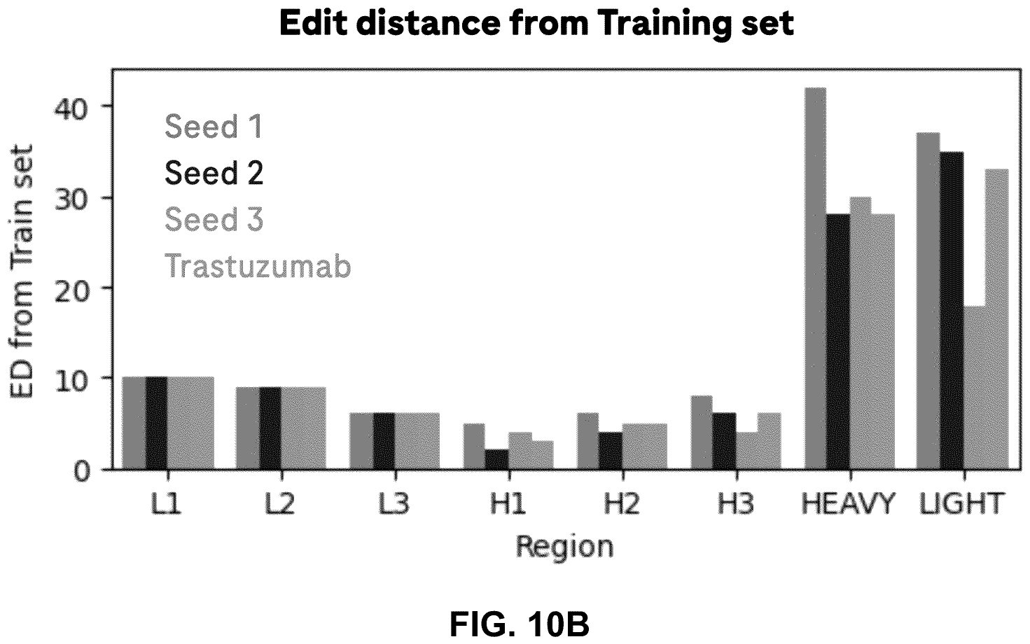

FIG. 10A depicts graphs illustrating the one-shot performance of a structure-informed molecule design computation model operating on a joint representation that combines the amino acid sequence of an input molecule with structural context information identifying, for each amino acid residue in the amino acid sequence, one or more adjacent amino acid residues in three-dimensional space. Two variants of the structure-informed molecule design computation model, one implemented with a graph transformer (e.g., a graph neural network (GNN)) and the other implemented with an autoencoder (e.g., a convolutional neural network (CNN) based autoencoder) were applied to improve the binding affinity of four input molecules, Seed 1, Seed 2, Seed 3, and Trastuzumab. The edit distance between the different complementarity determining regions of the heavy chain (H1, H2, and H3) and light chain (L1, L2, and L3) of four different input molecules, Seed 1, Seed 2, Seed 3, and Trastuzumab, relative to the matched dataset used for training are shown in FIG. 10B.

Referring again to FIG. 10A, in Panel A, the distribution edit distances between an out-of-distribution (OOD) input molecule and the output molecules generated therefrom using a sequence only molecule design computation model (PropEn) and two variants of the structure-informed molecule design computation model (Affinity Enhancer GNN and Affinity Enhancer CNN) are shown. As shown in Panel A, the sequence only molecule design computation model tend to generate output molecules that are too dissimilar to the out-of-distribution input molecule. Panel B shows the predicted binding count of unique designs from 5000 output molecules generated from the four input molecules by the structure-informed molecule design computation model. In Panel C, depicts a comparison of the binding affinity (measured in terms of pKD or the negative log 10 of the dissociation constant KD) present in the output molecules generated by the structure-informed molecule design computation model (Affinity Enhancer). Panel D depicts a comparison of the binding affinities of the output molecules generated by the structure-informed molecule design computation model (Affinity Enhancer) and a structured-conditioned molecule design computation model (Antifold). Panel E depicts the distribution of the improvement in binding affinity achieved by the structure-informed molecule design computation model (Affinity Enhancer) and the structured-conditioned molecule design computation model (Antifold).

### Iterative Training on Pseudo-Matched Molecule Pairs

In some example embodiments, the performance of a molecule design computation model, such as the molecule design computation model 115 shown in FIG. 1, may be improved through iterative training, in which successive training cycles leverages pseudo-matched (or synthetic) molecule pairs that includes output molecules generated by the molecule design computation model from previous training cycles. For example, in some cases, the performance of the conditional generator q_{θ}(x′|x) may be improved by leveraging pseudo-matched (or synthetic) molecule pairs, each of which having one molecule from an original dataset and another molecule that is generated by the conditional generator q_{θ}(x′|x) therefrom. It should be appreciated that iterative training with pseudo-matched molecule pairs may be applied when the molecule design computation model is being trained to generate one or more output molecules with superior values for one or more properties of interest by encoding an input molecule and decoding the resulting embedding of the input molecule. Alternatively, it is also possible to apply iterative training on pseudo-matched molecule pairs when training the molecule design computation model to generate the one or more output molecules by at least denoising a noise molecule while conditioned on the input molecule. Furthermore, it should be appreciated that iterative training on pseudo-matched molecule pairs may also be applied in instances where the molecule design computation model is trained to operate in a structure-informed manner, for example, by operating on a joint representation of the input molecule, which combines a linear (or one-dimensional) representation of the input molecule (e.g., the amino acid sequence of the input molecule) with a three-dimensional representation of the input molecule (e.g., an adjacency matrix identifying adjacent amino acid residues in three-dimensional space).

Algorithm 2 below describes the pseudo-code implementing the iterative training of the conditional generator q_{θ}(x′|x) on pseudo-matched (or synthetic) molecule pairs.

Algorithm 2: Iterative Pseudo-Matching

```
Data: Initial parameters θ⁽⁰⁾, data distribution p(x), n neighborhood radius
Δ_{ₓ}, property function g(·), iterations T, samples per round N, criterion [symbol] .
Result: Trained parameters θ^{(T)}.
for t ← 0 to T − 1 do
  // 1. Generate candidate samples
  Draw ${\{ {x}_{i} \}}_{i = 1}^{N} ∼ p ( x ) ;$
  Draw ${\{ {x}_{i}^{′} \}}_{i = 1}^{N} ∼ {q}_{{θ}^{( t )}} ( {x}^{′} \vert {x}_{i} ) ;$
  // 2. Filter by distance and property
  ${ℳ}^{( t )} ← \{ {x}_{i} , {x}_{i}^{′} : dist ( {x}_{i}^{′} - {x}_{i} ) \leq {Δ}_{x} ∧ g ( {x}_{i}^{′} ) > g ( xi ) \} ;$
  // 3. Retrain on pseudo-matches
  ${θ}^{( t + 1 )} ← {arg min}_{θ} 𝒥 ( θ , ℳ ) // arg {max}_{θ} in case of mVAE$
end for
return θ^{(T)}
```

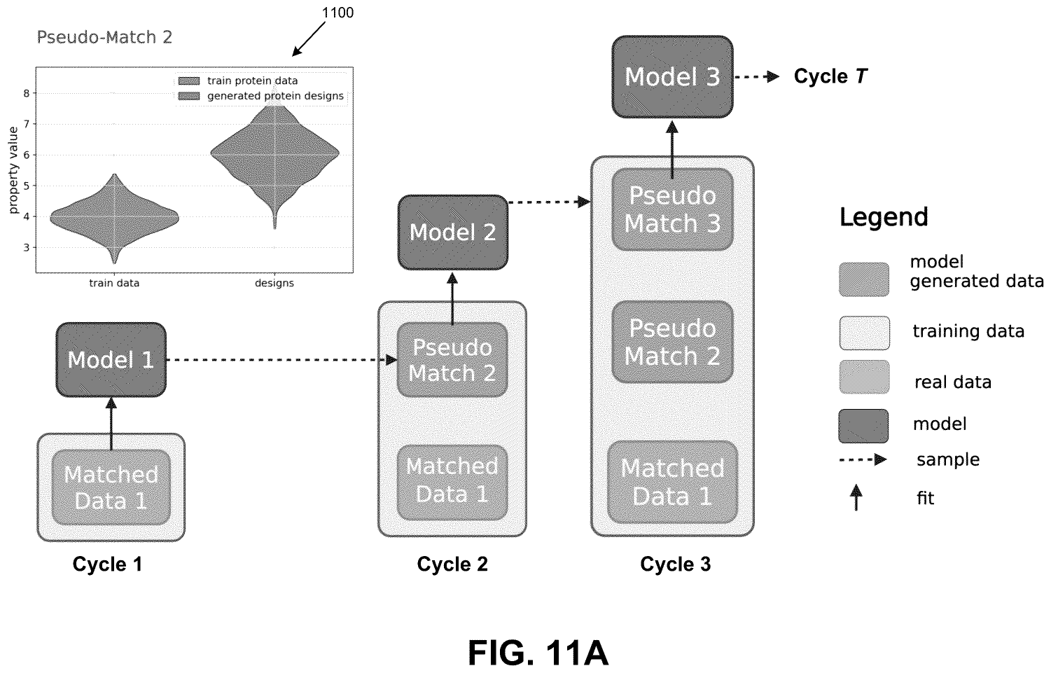

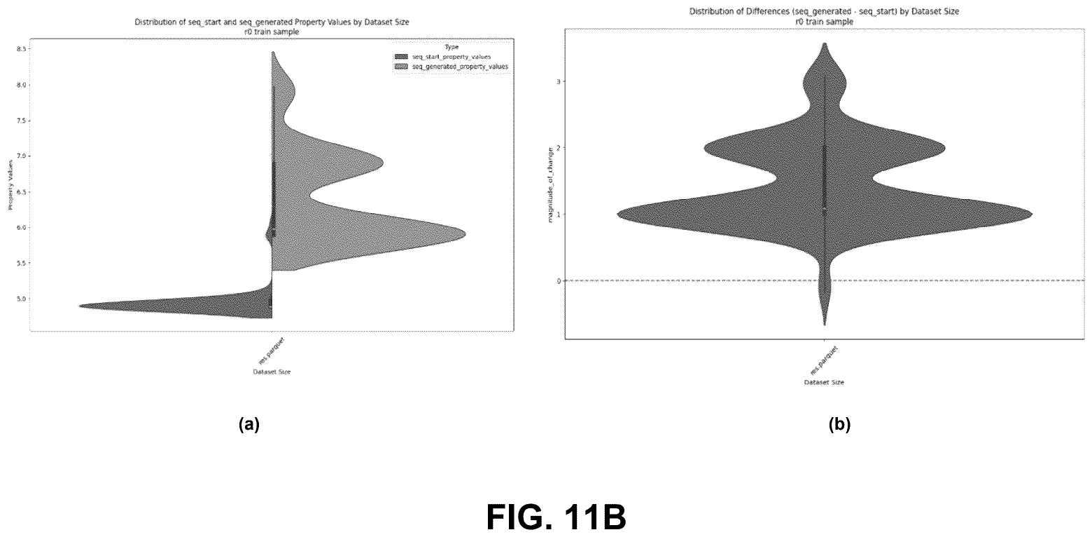

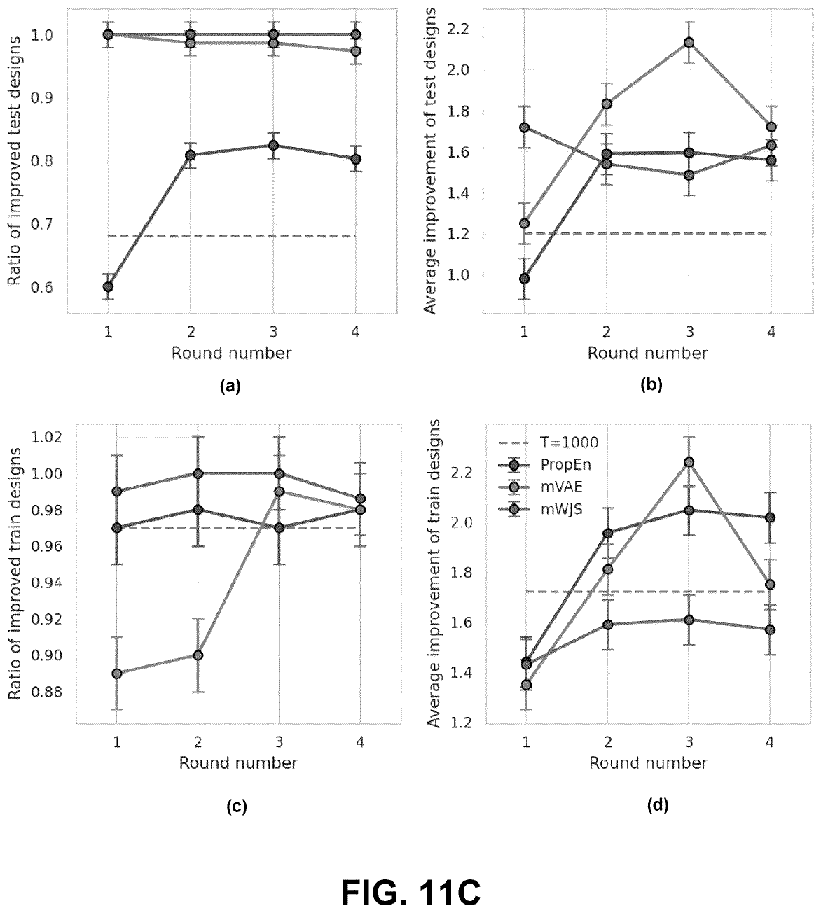

To further illustrate, the concept of iterative training using pseudo-matched (or synthetic) molecules is illustrated schematically in FIG. 11A. As shown in FIG. 11A, synthetic data from one training cycle are accumulated and paired with true molecules from the original matched dataset before being added to the training data for the next training cycle. For example, in FIG. 11A, during a first training cycle (“Cycle 1” in FIG. 11A), a first instance of a molecule design computation model (“Model 1” in FIG. 11A) may be trained on a matched dataset (“Matched Data 1” in FIG. 11A). The matched dataset (“Matched Data 1”) may include molecule pairs formed from “true” or “real” molecules. In this context, a “true” or “real” molecule may be a known molecule for which empirical measurements are available for one or more properties of the interest. In some cases, the first instance of the molecule design computation model (“Model 1”) may be trained on the matched dataset (“Matched Data 1”) to learn the causation (or dependency) between differences in molecular features, such as certain compositional features and/or conformational features, and the corresponding differences in the one or more properties of interest. For instance, in some cases, the first instance of the molecule design computation model (“Model 1”) may be trained to approximate the gradient of the one or more properties (e.g., a function predicting the value of the one or more properties) such that the trained molecule design computation model (“Model 1”) may generate one or more output molecules having superior values for the one or more properties of interest by at least encoding an input molecule and decoding the resulting embedding of the input molecule. Alternatively, the first instance of the molecule design computation model may be trained to approximate the data distribution (or matched distribution) of the molecule pairs in the matched dataset (“Matched Data 1”) such that the trained molecule design computation model may be applied to generate the one or more output molecules by at least denoising a noise molecule while conditioned on the input molecule.

Once the first instance of the molecule design computation model (“Model 1”) has been trained on the matched dataset (“Matched Data 1”), the first instance of the molecule design computation model may be applied to generate, based at least on the “true” or “real” molecules in the matched dataset (“Matched Data 1”), one or more output molecules having superior values for the one or more properties of interest. That is, in some cases, one or more of the “true” or “real” molecules from the matched dataset (“Matched Data 1”) may serve as input molecules upon which the first instance of the molecule design computation model may be applied to generate one or more output molecules with superior values for the one or more properties of interest. In some cases, the output molecules generated by the first instance of the molecule design computation model (“Model 1”) may be paired with the corresponding input molecules from the matched dataset (“Matched Data 1”) to generate pseudo-matched molecule pairs. Moreover, multiple pseudo-matched pairs generated in this manner may form a pseudo-matched dataset (“Pseudo Match 2” in FIG. 11A), which is then used along with the original matched dataset (“Matched Data 1”) to train a second instance of the molecule design computation model (“Model 2” in FIG. 11A) during a subsequent training cycle (“Cycle 2” in FIG. 11A). In this context, prior to undergoing any training, the first instance (“Model 1”) and the second instance of the molecule design computation model (“Model 2”) may have the same architecture and initial parameters (e.g., weights, biases, and/or the like). In some example embodiments of iterative training, instead of further training the first instance of the molecule design computation model (“Model 1”) with the pseudo-matched dataset (“Pseudo Match 2”), the second instance of the molecule design computation model (“Model 2”) may be trained from scratch (e.g., from initial parameters) using the combination of the original matched dataset (“Matched Data 1”) and the pseudo-matched dataset (“Pseudo Match 2”). In some cases, the second instance of the molecule design computation model (“Model 2”) may be generated by re-initializing (e.g., to initial values) the parameters (e.g., weights, biases, and/or the like) of the first instance of the molecule design computation model (“Model 1”) after the first instance of the molecule design computation model (“Model 1”) has been trained and applied to generate the pseudo-matched dataset (“Pseudo Match 2”).

In some cases, additional cycles of iterative training with pseudo-matched molecule pairs may be performed (e.g., up to a T quantity of training cycles). For example, in the example shown in FIG. 11A, a third training cycle (“Cycle 3” in FIG. 11A) may be performed in which a third instance of the molecule design computation model (“Model 3” in FIG. 11A) is trained using the original matched dataset (“Matched Data 1”), the pseudo-matched dataset (“Matched Data 1”) from the first training cycle (“Cycle 1”), and an additional pseudo-matched dataset (“Matched Data 2”) from the second training cycle (“Cycle 2”). In some cases, the additional pseudo-matched dataset (“Matched Data 2”) may be generated by pairing the “real” or “true” molecules in the original matched dataset (“Matched Data 1”) with the output molecules generated by applying the trained second instance of the molecule design computation model (“Model 2”) to those “real” or “true” molecules in the original matched dataset (“Matched Data 1”). As shown in the violin chart comparing the property values of the molecules in the training data and that of the output molecules generated by the molecule design computation model, the output molecules may exhibit superior values for one or more properties of interest. Accordingly, the values of the properties present in the output molecules generated by the molecule design computation model may be pushed incrementally higher with each successive cycle of iterative training.

In some cases, iterative training in this manner augments the size of the matched dataset, which may often contain a limited quantity of “real” or “true” molecules, with pseudo-matched (or synthetic) molecule pairs. As noted, in some cases, iterative training may push the values of the properties present in the output molecules beyond what is present in the original matched dataset. To demonstrate the effects of iterative training empirically, a matched walk-jump sampler (mWJS) variation of the conditional generator q_{θ}(x′|x) was trained on one-hot encoded protein sequences with 298 amino acid residue to maximize physico-chemical properties. In Panel (a) of FIG. 11B, the violin plot illustrate the distribution of the property values present in the input molecules in the original dataset and in the output molecules generated by the conditional generator q_{θ}(x′|x) across different sizes of the original dataset. Panel (b) shows a pronounced rightward shift in the property values of the output distribution, yielding pseudo-matched (input, output) molecule pairs that were then fed back into the conditional generator q_{θ}(x′|x) for the next training cycle. The distribution of the difference (or magnitude of change) between the input molecules in the original dataset and the output molecules generated therefrom are show in Panel (b) of FIG. 11B.

FIG. 11C further demonstrates the effects of iterative training on an implicitly guided property optimizer (PropEn in FIG. 11C) and two variants of the conditional generator q_{θ}(x′|x) (mVAE and mWJS in FIG. 11C) in terms of the ration of improved output molecules (Panel (a)), the average improvement of output molecules (Panel (b)), the ratio of improved input molecules (Panel (c)), and the average improvement of input molecules (Panel (d)). Starting with 400 input molecules (or seed molecules), each successive training cycle outperforms (by the second training cycle) a baseline model trained on 1,000 ground-truth molecule pairs, in both the fraction of output molecules with superior property values and in average gain in property value. Moreover, in each training cycle, the property ceiling (or maximum gain in property value) rises until it plateaus around the fourth training cycle, which is consistent with the initial dataset.

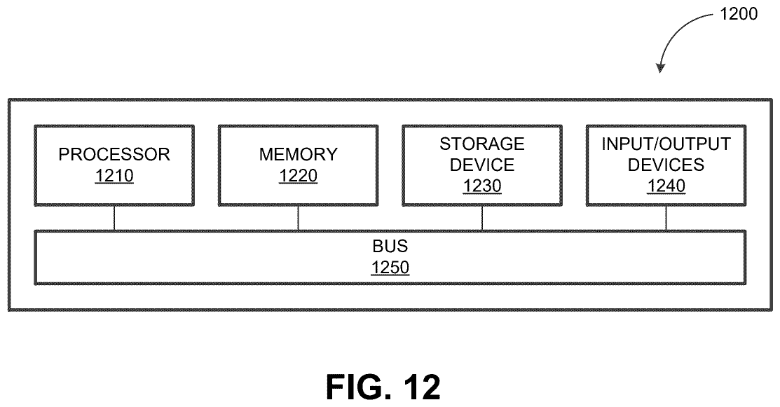

FIG. 12 depicts a block diagram illustrating an example of a computing system 1200, in accordance with some example embodiments. Referring to FIGS. 1-12, the computing system 1200 may be used to implement the molecule design engine 110, the selection engine 120, the laboratory equipment 130, the client device 140, and/or any components therein.

As shown in FIG. 12, the computing system 1200 can include a processor 1210, a memory 1220, a storage device 1230, and input/output devices 1240. The processor 1210, the memory 1220, the storage device 1230, and the input/output devices 1240 can be interconnected via a system bus 1250. The processor 1210 is capable of processing instructions for execution within the computing system 1200. Such executed instructions can implement one or more components of, for example, the molecule design engine 110, the selection engine 120, the laboratory equipment 130, the client device 140, and/or the like. In some example embodiments, the processor 1210 can be a single-threaded processor. Alternately, the processor 1210 can be a multi-threaded processor. The processor 1210 is capable of processing instructions stored in the memory 1220 and/or on the storage device 1230 to display graphical information for a user interface provided via the input/output device 1240.

The memory 1220 is a computer readable medium such as volatile or non-volatile that stores information within the computing system 1200. The memory 1220 can store data structures representing configuration object databases, for example. The storage device 1230 is capable of providing persistent storage for the computing system 1200. The storage device 1230 can be a floppy disk device, a hard disk device, an optical disk device, or a tape device, or other suitable persistent storage means. The input/output device 1240 provides input/output operations for the computing system 1200. In some example embodiments, the input/output device 1240 includes a keyboard and/or pointing device. In various implementations, the input/output device 1240 includes a display unit for displaying graphical user interfaces.

According to some example embodiments, the input/output device 1240 can provide input/output operations for a network device. For example, the input/output device 1240 can include Ethernet ports or other networking ports to communicate with one or more wired and/or wireless networks (e.g., a local area network (LAN), a wide area network (WAN), the Internet).

In some example embodiments, the computing system 1200 can be used to execute various interactive computer software applications that can be used for organization, analysis and/or storage of data in various formats. Alternatively, the computing system 1200 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1240. The user interface can be generated and presented to a user by the computing system 1200 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by the at least one data processor, result in operations comprising:
identifying an input molecule exhibiting a value for one or more properties; and
applying a molecule design computation model to generating one or more output molecules exhibiting a different value for the one or more properties than the input molecule, wherein the molecule design computation model generates the one or more molecules by at least
encoding the input molecule to generate an embedding of the input molecule, and
decoding the embedding of the input molecule to generate the one or more output molecules.

2. The system of claim 1, wherein the generating one or more output molecules includes:
applying the molecule design computation model to generate an output molecule;
determining that the output molecule fails to satisfy one or more criteria; and
in response to determining that the output molecule fails to satisfy the one or more criteria, applying the molecule design computation model to generate an additional output molecule.

3. The system of claim 2, wherein the molecule design computation model generates the additional output molecule by at least
encoding the output molecule to generate an embedding of the output molecule, and
decoding the embedding of the output molecule to generate the additional output molecule.

4. The system of claim 2, wherein the one or more criteria include (i) a proximity measure between the input molecule and the output molecule satisfying a first threshold, and (ii) a difference in the value of the one or more properties present in the input molecule and the different value of the one or more properties present in the output molecule satisfying a second threshold.

5. The system of claim 1, further comprising:
identifying, for inclusion in a training dataset, a plurality of molecule pairs, wherein each molecule pair includes two molecules exhibiting different values for the one or more properties; and
training, based at least on the training dataset, the molecule design computation model to generate, based at least on a first molecule in each molecule pair, a reconstruction of a second molecule in each molecule pair.

6. The system of claim 5, wherein the molecule design computation model is trained, based at least on the training dataset, to at least
encode the first molecule to generate an embedding of the first molecule, and
decode the embedding of the first molecule to generate the reconstruction of the second molecule.

7. The system of claim 5, wherein the training of the molecule design computation model includes reducing a reconstruction loss associated with a difference between the second molecule and the reconstruction of the second molecule generated by the molecule design computation model.

8. The system of claim 5, wherein the training of the molecule design computation model includes imposing a monotonicity constraint by at least ensuring that a first output of the molecule design computation model operating on the first molecule is greater than a second output of the molecule design computation model operating on the second molecule where the first molecule is greater than the second molecule.

9. The system of claim 5, wherein each molecule pair is identified by at least identifying, based at least on one or more criteria being satisfied, the first molecule as a match for the second molecule.

10. The system of claim 9, further comprising:
determining that the one or more criteria are satisfied based at least on (i) a proximity measure between the first molecule and the second molecule satisfying one or more thresholds, and (ii) a difference in a value of the one or more properties present in the first molecule and a value of the one or more properties present in the second molecule satisfying one or more thresholds.

11. (canceled)

12. (canceled)

13. The system of claim 9, wherein the one or more properties include a first property and a second property.

14. The system of claim 13, further comprising:
determining that the one or more criteria are satisfied based at least on a difference in a respective value of either the first property or a second property present in each of the first molecule and the second molecule satisfying one or more thresholds.

15. The system of claim 13, further comprising:
determining, for each of the first molecule and the second molecule, a multivariate rank indicative of a difference in a combination of the first property and the second property; and
determining that the one or more criteria are satisfied based at least on a difference in a respective multivariate rank of the first molecule and the second molecule satisfying one or more thresholds.

16. (canceled)

17. The system of claim 13, wherein the first property and the second property comprise a different one of binding affinity, binding specificity, hydrophobicity, size of electrical charge patches, angle delta, angle length, immunogenicity, and presence of liability motifs.

18. (canceled)

19. (canceled)

20. (canceled)

21. The system of claim 1, wherein the input molecule comprises a protein sequence, and wherein the output molecule comprises a different protein sequence.

22. The system of claim 1, wherein the input molecule comprises a nucleic acid molecule, and wherein the output molecule comprises a nucleic acid molecule having a different sugar-phosphate backbone than the input molecule.

23. The system of claim 1, wherein the input molecule comprises a chemical compound, and wherein the output molecule comprises a chemical compound having one or more different functional groups than the input molecule.

24. (canceled)

25. The system of claim 1, wherein the molecule design computation model generates an output comprising a multinomial distribution of a plurality of possible composition and/or a plurality of conformation of the one or more output molecules.

26. (canceled)

27. The system of claim 25, wherein the multinomial distribution includes, for each possible position in a protein sequence, a probability of the position being occupied by each of a plurality of possible amino acid residues, wherein the sampling from the multinomial distribution includes determining, based on the multinomial distribution, a type of amino acid residue occupying each position in a corresponding protein sequence, and wherein the type of amino acid residue determined to occupy a position in the corresponding protein sequences comprises a type of amino acid residue whose probability of occupying the position satisfies one or more thresholds.

28. (canceled)

29. (canceled)

30. (canceled)

31. A system, comprising:
at least one data processor; and
at least one memory storing instructions, which when executed by the at least one data processor, result in operations comprising:
identifying, for inclusion in a matched dataset, a plurality of molecule pairs, wherein each molecule pair of the plurality of molecule pairs include two molecules exhibiting different values for one or more properties;
training, based at least on the matched dataset, a molecule design computation model to generate, based at least on a first molecule in each molecule pair, a reconstruction of a second molecule in each molecule pair, wherein the molecule design computation model is trained to generate the reconstruction of the second molecule by at least
encoding the first molecule to generate an embedding of the first molecule, and
decoding the embedding of the first molecule to generate the reconstruction of the second molecule; and
applying the molecule design computation model to generate, based at least on an input molecule, one or more output molecules having a different value for the one or more properties than the input molecule.

32. The system of claim 31, further comprising:
identifying each molecule pair by at least identifying, based at least on one or more criteria, the first molecule as a match for the second molecule.

33. The system of claim 32, further comprising:
determining that the one or more criteria are satisfied based at least on a proximity measure between the first molecule and the second molecule satisfying one or more thresholds.

34. (canceled)

35. The system of claim 32, further comprising:
determining that the one or more criteria are satisfied based at least on a difference in a value of the one or more properties present in the first molecule and a value of the one or more properties present in the second molecule satisfying one or more thresholds.

36. The system of claim 32, wherein the one or more properties include a first property and a second property.

37. The system of claim 36, further comprising:
determining that the one or more criteria are satisfied based at least on a difference in a respective value of either the first property or a second property present in each of the first molecule and the second molecule satisfying one or more thresholds.

38. The system of claim 36, further comprising:
determining, for each of the first molecule and the second molecule, a multivariate rank indicative of a difference in a combination of the first property and the second property; and
determining that the one or more criteria are satisfied based at least on a difference in a respective multivariate rank of the first molecule and the second molecule satisfying one or more thresholds.

39. (canceled)

40. The system of claim 36, wherein the first property and the second property comprise a different one of binding affinity, binding specificity, hydrophobicity, size of electrical charge patches, angle delta, angle length, immunogenicity, and presence of liability motifs.

41. (canceled)

42. (canceled)

43. A computer-implemented method, comprising:
identifying an input molecule exhibiting a value for one or more properties; and
applying a molecule design computation model to generating one or more output molecules exhibiting a different value for the one or more properties than the input molecule, wherein the molecule design computation model generates the one or more molecules by at least
encoding the input molecule to generate an embedding of the input molecule, and
decoding the embedding of the input molecule to generate the one or more output molecules.

44. A computer-implemented method, comprising:
identifying, for inclusion in a matched dataset, a plurality of molecule pairs, wherein each molecule pair of the plurality of molecule pairs include two molecules exhibiting different values for one or more properties;
training, based at least on the matched dataset, a molecule design computation model to generate, based at least on a first molecule in each molecule pair, a reconstruction of a second molecule in each molecule pair, wherein the molecule design computation model is trained to generate the reconstruction of the second molecule by at least
encoding the first molecule to generate an embedding of the first molecule, and
decoding the embedding of the first molecule to generate the reconstruction of the second molecule; and
applying the molecule design computation model to generate, based at least on an input molecule, one or more output molecules having a different value for the one or more properties than the input molecule.

45. The system of claim 10, wherein the proximity measure includes one or more of an edit distance, a structural similarity, an amino acid substitution matrix, a chemical similarity coefficient, a Euclidean distance, atomic coordinates, torsion angles, and an embedding of each of the first molecule and the second molecule
